(19) United States
(12) Patent Application Publication
(10) Pub. No.: US 2024/0274237 A1
(43) Pub. Date: Aug. 15, 2024

(54) BARCODE SELECTION

(71) Applicant: ULTIMA GENOMICS, INC., Fremont, CA (US)

(72) Inventors: Yoav ETZIONI, Tel Aviv (IL); Omer BARAD, Mazkeret Batya (IL); Florian OBERSTRASS, Menlo Park, CA (US); Edward PERELMAN, Lehavim (IL); Mark GESHEL, Kfar Saba (IL)

(21) Appl. No.: 18/410,051

(22) Filed: Jan. 11, 2024

Related U.S. Application Data
(63) Continuation of application No. PCT/US2022/037204, filed on Jul. 14, 2022.
(60) Provisional application No. 63/221,513, filed on Jul. 14, 2021.

(51) Int. Cl. G16B 30/00; G16B 45/00

(52) U.S. Cl. CPC G16B 30/00; C12Q 1/6876; G16B 45/00; C12Q 1/6869; G16B 35/00

(57) ABSTRACT

Provided herein are methods, systems, and compositions for generating and selecting barcode sequences. A method for selecting barcode sequences may comprise generating a set of sequence data for the barcode sequences and filtering the data using one or more criteria or filters to provide a filtered set of barcode sequences. The resultant filtered set of barcode sequences may satisfy one or more selection criteria and may be sufficiently diverse from one another.

### CROSS-REFERENCE

This application is a continuation of International Patent Application No. PCT/US2022/037204, filed Jul. 14, 2022, which claims benefit of U.S. Provisional Application No. 63/221,513, filed Jul. 14, 2021, the contents of which are incorporated herein by reference in its entirety.

### SEQUENCE LISTING

The instant application contains a Sequence Listing which has been submitted electronically in XML format and is hereby incorporated by reference in its entirety. Said XML copy, created on Oct. 17, 2022, is named 51024-761_301_SL.xml and is 1.05 million bytes in size.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification.

Barcode sequences may be used in identifying or distinguishing a nucleic acid molecule from another nucleic acid molecule. For example, nucleic acid molecules having different barcode sequences may be used to label or identify a sample origin, location, etc.

Despite the advance of sequencing technology and the use of nucleic acid barcode molecules, selecting barcode sequences for use in a system may be laborious or result in poor separation performance. For example, barcode molecules having similar sequences may be difficult to distinguish from one another.

### SUMMARY

Recognized herein is a need for producing sufficiently diverse nucleic acid barcode sequences. Such sufficiently diverse barcode sequences may be useful in preparation of samples, analysis of nucleic acid molecules, and may be useful in providing improved attribution of a barcoded product to an origin (e.g., sample, partition, cell, etc.).

In an aspect, provided herein is a composition, comprising a non-naturally occurring nucleic acid barcode molecule comprising a sequence of any one of SEQ ID NOs: 1-1256.

In some embodiments, the non-naturally occurring nucleic acid barcode molecule is coupled to a support. In some embodiments, the support is a bead. In some embodiments, the support comprises one or more sequences selected from the group consisting of SEQ ID NOs: 1-1256. In some embodiments, the support comprises one or more sequences selected from the group consisting of SEQ ID NOs: 1-238. In some embodiments, the support comprises one or more sequences selected from the group consisting of SEQ ID NOs: 239-1256. In some embodiments, the non-naturally occurring nucleic acid barcode molecule comprises a sequence of any one of SEQ ID NOs: 1-238. In some embodiments, the non-naturally occurring nucleic acid barcode molecule comprises a sequence of any one of SEQ ID NOs: 239-1256. In some embodiments, the composition comprises a plurality of non-naturally occurring nucleic acid barcode molecules comprising at least 96 different sequences selected from the group consisting of SEQ ID NOs: 1-1256. In some embodiments, the composition comprises a plurality of non-naturally occurring nucleic acid barcode molecules comprising at least 96 different sequences selected from the group consisting of SEQ ID NOs: 1-238. In some embodiments, the composition comprises a plurality of non-naturally occurring nucleic acid barcode molecules comprising at least 96 different sequences selected from the group consisting of SEQ ID NOs: 239-1256.

In another aspect, provided herein is a computer-implemented method for generating or selecting a set of barcode sequences, comprising: (a) providing, by at least one processor, a plurality of barcode sequences; (b) generating, by the at least one processor, a plurality of matrices of flow data, wherein each matrix of the plurality of matrices of flow data corresponds to a different barcode sequence of the plurality of barcode sequences, and wherein a given matrix of flow data comprises information on a plurality of flow cycles that is representative of nucleotide incorporation events corresponding to a given barcode sequence of the plurality of barcode sequences; (c) applying, by the at least one processor, one or more constraints on the plurality of matrices of flow data, thereby generating a first set of filtered matrices; (d) filtering, by the at least one processor, the first set of filtered matrices using one or more criterions to generate a third set of filtered matrices corresponding to the set of barcode sequences, wherein the set of barcode sequences is a subset of barcode sequences of the plurality of barcode sequences; and (e) electronically outputting the set of barcode sequences.

In some embodiments, each barcode sequence of the set of barcode sequences is from 9 to 30 nucleotides in length. In some embodiments, each barcode sequence of the set of barcode sequences is from 9 and 11 nucleotides in length. In some embodiments, the plurality of matrices of flow data comprises a 1×N vector, and N is a number of flow cycles in the plurality of flow cycles. In some embodiments, the one or more criterions comprises barcode sequence length, and the filtering in (c) comprises removing matrices corresponding to barcode sequences that have a sequence length that is greater or less than a predetermined threshold value, thereby yielding a second set of filtered matrices. In some embodiments, a given matrix of the plurality of matrices of flow data, the first set of filtered matrices, or the second set of filtered matrices comprises a 1×N vector, and N is a number of flow cycles in the plurality of flow cycles, and each element of the 1×N vector is an H-mer representative of the nucleotide incorporation events, and H corresponds to a number of nucleotides incorporated per flow cycle of the plurality of flow cycles. In some embodiments, (c) further comprises calculating, using the at least one processor, an edit distance between the given matrix and another matrix of the plurality of matrices of flow data, the first set of filtered matrices, or the second set of filtered matrices, and the one or more criterions in (d) comprise a predetermined threshold or a range of edit distances. In some embodiments, the edit distance is calculated by counting, using the at least one processor, a number of different elements between two matrices of the second set of filtered matrices. In some embodiments, the predetermined threshold or the range of edit distances is at least 2. In some embodiments, the predetermined threshold or the range of edit distances is at least 4. In some embodiments, the one or more constraints in (b) comprises a minimum, a maximum, or a range of one or more parameters selected from the group consisting of: the number of flow cycles, H-mer magnitude, and a number of H-mers above a predetermined threshold H value. In some embodiments, the predetermined threshold H value is 7. In some embodiments, the electronically outputting in (e) comprises presenting, on a user interface, the set of barcode sequences.

Another aspect of the present disclosure provides a kit, comprising: at least 96 non-naturally occurring nucleic acid barcode molecules, and each of the at least 96 non-naturally occurring nucleic acid barcode molecules comprises a different sequence selected from the group consisting of SEQ ID NOs: 1-1256.

Another aspect of the present disclosure provides a kit, comprising: at least 96 non-naturally occurring nucleic acid barcode molecules, and each of the at least 96 non-naturally occurring nucleic acid barcode molecules comprises a different sequence selected from the group consisting of SEQ ID NOs: 1-238.

Another aspect of the present disclosure provides a kit, comprising: at least 96 non-naturally occurring nucleic acid barcode molecules, and each of the at least 96 non-naturally occurring nucleic acid barcode molecules comprises a different sequence selected from the group consisting of SEQ ID NOs: 239-1256.

Another aspect of the present disclosure provides a composition, comprising a non-naturally occurring nucleic acid barcode molecule consisting of 10-30 linked nucleotides, and the non-naturally occurring nucleic acid barcode molecule comprises a sequence comprising at least 8 contiguous nucleotides selected from the group consisting of SEQ ID NOs: 1-238.

Another aspect of the present disclosure provides a composition, comprising a non-naturally occurring nucleic acid barcode molecule consisting of 10-30 linked nucleotides, and the non-naturally occurring nucleic acid barcode molecule comprises a sequence comprising at least 8 contiguous nucleotides selected from the group consisting of SEQ ID NOs: 239-1256.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example flow sequencing method that can be used to generate sequencing data for a sample sequence (SEQ ID NO: 1257), in accordance with some embodiments.

FIG. 2A illustrates an example summary of detected signals after a number of example flow cycles are performed, in accordance with some embodiments.

FIG. 2B illustrates an example process for determining a preliminary sequence, in accordance with some embodiments.

FIG. 3 shows an example of a computing device that may be used to implement a method as described herein, in accordance with some embodiments.

FIG. 4 shows an example histogram of barcodes generated as a function of barcode sequence length.

FIG. 5 shows example data of number of barcodes generated as a function of barcode length.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Provided herein are methods, systems, compositions, and kits for generating or selecting a set of barcode sequences comprising a plurality of barcode sequences that are distinguishable (e.g., have high separation performance) from one another. Such barcode sequences may be useful in the preparation of samples, and/or for analysis or characterization of analytes (e.g., nucleic acids, proteins, lipids, carbohydrates), e.g., via sequencing. For example, the methods and systems described herein may be used to generate or select barcode sequences that may be used in nucleic acid sequencing. In such cases, it may be useful to utilize barcode sequences that are sufficiently distinct from one another, such that a single barcode sequence can be uniquely traced to a particular sample, origin, partition, etc. Using distinct barcode sequences may also reduce errors (e.g., caused by overlapping barcode sequences, barcode sequences that are too similar that they cannot be distinguished), such as during sample analysis or characterization (e.g., sequencing). The barcode sequences may further be generated or selected based on one or more criteria, e.g., barcode sequence length, number of flow cycles (as described elsewhere herein) to generate the entire barcode sequence read, etc.

The term “biological sample,” as used herein, generally refers to any sample from a subject or specimen. The biological sample can be a fluid or tissue from the subject or specimen. The fluid can be blood (e.g., whole blood), saliva, urine, or sweat. The tissue can be from an organ (e.g., liver, lung, or thyroid), or a mass of cellular material, such as, for example, a tumor. The biological sample can be a feces sample, collection of cells (e.g., cheek swab), or hair sample. The biological sample can be a cell-free or cellular sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid molecules may be cell-free or cell-free nucleic acid molecules, such as cell free DNA or cell free RNA. The nucleic acid molecules may be derived from a variety of sources including human, mammal, non-human mammal, ape, monkey, chimpanzee, reptilian, amphibian, avian, or plant sources. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself.

The term “subject,” as used herein, generally refers to an individual from whom a biological sample is obtained. The subject may be a mammal or non-mammal. The subject may be an animal, such as a monkey, dog, cat, bird, or rodent. The subject may be a human. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, or cervical cancer) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or deoxyribonucleic acids (DNA) or ribonucleotides or ribonucleic acids (RNA), or analogs thereof. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid molecule can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), or more. A nucleic acid molecule (e.g., polynucleotide) can comprise a sequence of four natural nucleotide bases: adenine (A); cytosine (C); guanine (G); and thymine (T) (uracil (U) for thymine (T) when the polynucleotide is RNA). A nucleic acid molecule may include one or more nonstandard nucleotide(s), nucleotide analog(s) and/or modified nucleotide(s). The term “nucleoside,” as used herein, generally refers to a nucleotide base lacking a phosphate group (e.g., adenine instead of adenosine).

The term “nucleotide,” as used herein, generally refers to any nucleotide or nucleotide analog. The nucleotide may be naturally occurring or non-naturally occurring. The nucleotide analog may be a modified, synthesized or engineered nucleotide. The nucleotide analog may not be naturally occurring or may include a non-canonical base. The naturally occurring nucleotide may include a canonical base. The nucleotide analog may include a modified polyphosphate chain (e.g., triphosphate coupled to a fluorophore). The nucleotide analog may comprise a label. The nucleotide analog may be terminated (e.g., reversibly terminated). The nucleotide analog may comprise an alternative base.

Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thiotriphosphate and beta-thiotriphosphate) or modifications with selenium moieties (e.g., phosphoroselenoate nucleic acids). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotide analogs may be capable of reacting or bonding with detectable moieties for nucleotide detection.

Nonstandard nucleotides, nucleotide analogs, and/or modified analogs may be terminated (e.g., reversibly terminated). For example, a nucleotide may comprise a reversible terminator, or a moiety that is capable of terminating primer extension reversibly. Nucleotides comprising reversible terminators may be accepted by polymerases and incorporated into growing nucleic acid sequences analogously to non-reversibly terminated nucleotides. A polymerase may be any naturally occurring (i.e., native or wild-type) or engineered variant of a polymerase (e.g., DNA polymerase, Taq polymerase, etc.). Following incorporation of a nucleotide analog comprising a reversible terminator into a nucleic acid strand, the reversible terminator may be removed to permit further extension of the nucleic acid strand. A reversible terminator may comprise a blocking or capping group that is attached to the 3-oxygen atom of a sugar moiety (e.g., a pentose) of a nucleotide or nucleotide analog. Such moieties are referred to as 3′-O-blocked reversible terminators. Examples of 3′-O-blocked reversible terminators include, for example, 3′-ONH2 reversible terminators, 3′-O-allyl reversible terminators, and 3′-O-aziomethyl reversible terminators. Alternatively, a reversible terminator may comprise a blocking group in a linker (e.g., a cleavable linker) and/or dye moiety of a nucleotide analog. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp. and the “lightning terminator” developed by Michael L. Metzker et al. Cleavage of a reversible terminator may be achieved by, for example, irradiating a nucleic acid molecule including the reversible terminator. In some instances, the plurality of nucleotides may not comprise a terminated nucleotide.

Nonstandard nucleotides, nucleotide analogs, and/or modified analogs may be labeled with a dye, fluorophore, or quantum dot. For example, the solution may comprise labeled nucleotides. In another example, the solution may comprise unlabeled nucleotides. In another example, the solution may comprise a mixture of labeled and unlabeled nucleotides. Non-limiting examples of dyes include SYBR green, SYBR blue, DAPI, propidium iodine, Hoechst, SYBR gold, ethidium bromide, acridine, proflavine, acridine orange, acriflavine, fluorocounarin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, Atto 390, 425, 465, 488, 495, 532, 565, 594, 633, 647, 647N, 665, 680 and 700 dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare); Dy-Quenchers (Dyomics), such as DYQ-660 and DYQ-661; and ATTO fluorescent quenchers (ATTO-TEC GmbH), such as ATTO 540Q, 580Q, 612Q. In some cases, the label may be one with linkers. For instance, a label may have a disulfide linker attached to the label. Non-limiting examples of such labels include Cy5-azide, Cy-2-azide, Cy-3-azide, Cy-3.5-azide, Cy5.5-azide and Cy-7-azide. In some cases, a linker may be a cleavable linker. In some cases, the label may be a type that does not self-quench or exhibit proximity quenching. Non-limiting examples of a label type that does not self-quench or exhibit proximity quenching include Bimane derivatives such as Monobromobimane. Alternatively, the label may be a type that self-quenches or exhibits proximity quenching. Non-limiting examples of such labels include Cy5-azide, Cy-2-azide, Cy-3-azide, Cy-3.5-azide, Cy5.5-azide and Cy-7-azide. In some instances, a blocking group of a reversible terminator may comprise the dye.

The term “analyte” may refer to molecules, cells, biological particles, or organisms. In some instances, a molecule may be a nucleic acid molecule, antibody, antigen, peptide, protein, or other biological molecule obtained from or derived from a biological sample. An analyte may originate from, and/or be derived from, a sample, such as a biological sample, such as from a cell or organism. An analyte may be synthetic. An analyte may be a biological analyte. For instance, the biological analyte may be a macromolecule (e.g., a nucleic acid, a carbohydrate, a protein, a lipid, etc.). The biological analyte may comprise multiple macromolecular groups (e.g., glycoproteins, proteoglycans, ribozymes, liposomes, etc.). The biological analyte may be an antibody, antibody fragment, or engineered variant thereof, an antigen, a cell, a peptide, a polypeptide, etc. In some cases, the biological analyte comprises a nucleic acid molecule. The nucleic acid molecule may comprise at least about 10, 100, 1000, 10,000, 100,000, 1,000,000, 10,000,000, 100,000,000, 1,000,000,000 or more nucleotides. Alternatively or in addition, the nucleic acid molecule may comprise at most about 1,000,000,000, 100,000,000, 10,000,000, 1,000,000, 100,000, 10,000, 1000, 100, 10 or fewer nucleotides. The nucleic acid molecule may have a number of nucleotides that is within a range defined by any two of the preceding values. In some cases, the nucleic acid molecule may also comprise a common sequence, to which an N-mer may bind. An N-mer may comprise 1, 2, 3, 4, 5, or 6 nucleotides and may bind the common sequence. In some cases, the nucleic acid molecules may be amplified to produce a colony of nucleic acid molecules attached to the substrate or attached to beads that may associate with or be immobilized to the substrate. In some instances, the nucleic acid molecules may be attached to beads and subjected to a nucleic acid reaction, e.g., amplification, to produce a clonal population of nucleic acid molecules attached to the beads.

The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The term “sequencing,” as used herein, generally refers to a process for generating or identifying a sequence of a biological molecule, such as a nucleic molecule. Such sequence may be a nucleic acid sequence, which may include a sequence of nucleic acid bases. Sequencing may be single molecule sequencing or sequencing by synthesis, for example. Sequencing may be performed using analyte nucleic acid molecules immobilized on a support, such as a flow cell or one or more beads. In some cases, sequencing may comprise generating sequencing signals and/or sequencing reads from the analyte nucleic acid molecules.

The terms “amplifying,” “amplification,” and “nucleic acid amplification” are used interchangeably herein and generally refer to generating one or more copies of a nucleic acid or a template. For example, “amplification” of DNA generally refers to generating one or more copies of a DNA molecule. Moreover, amplification of a nucleic acid may be linear, exponential, or a combination thereof. Amplification may be emulsion based or may be non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Moreover, amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C.C. PNAS, 1989, 86, 4076-4080 and U.S. Pat. Nos. 5,409,811 and 5,674,716, each of which is herein incorporated by reference in its entirety.

Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference).

The term “detector,” as used herein, generally refers to a device that is capable of detecting a signal, including a signal indicative of the presence or absence of one or more incorporated nucleotides or fluorescent labels. The detector may detect multiple signals. The signal or multiple signals may be detected in real-time during, substantially during a biological reaction, such as a sequencing reaction (e.g., sequencing during a primer extension reaction), or subsequent to a biological reaction. In some cases, a detector can include optical and/or electronic components that can detect signals. The term “detector” may be used in detection methods. Non-limiting examples of detection methods include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering, Rayleigh scattering, Raman scattering, surface-enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products. A detector may be a continuous area scanning detector. For example, the detector may comprise an imaging array sensor capable of continuous integration over a scanning area wherein the scanning is electronically synchronized to the image of an object in relative motion. A continuous area scanning detector may comprise a time delay and integration (TDI) charge coupled device (CCD), Hybrid TDI, or complementary metal oxide semiconductor (CMOS) pseudo TDI device. For example, a continuous area scanning detector may comprise a TDI line-scan camera.

The term “nucleotide incorporation event”, as used herein, generally refers to the incorporation of a nucleotide into a growing strand of a nucleic acid molecule in the presence or absence of a nucleic acid template.

The term “open substrate,” as used herein, generally refers to a substrate in which any point on an active surface of the substrate is physically accessible from a direction normal to the substrate. The systems and methods for sequencing in accordance with disclosure herein may utilize a substrate comprising a plurality of individually addressable locations. The plurality of individually addressable locations may be arranged as an array on the substrate. The plurality of individually addressable locations may be otherwise arranged, such as randomly or in any order, on the substrate. Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). For example, an analyte or reagent may be immobilized to an individually addressable location via a support, such as a bead. In some instances, a bead is immobilized to the individually addressable location, and the analyte or reagent is immobilized to the bead. In some cases, an individually addressable location may immobilize thereto a plurality of analytes or a plurality of reagents. The plurality of analytes may be copies of a template analyte. For example, the plurality of analytes may have sequence homology or sequence identity. For example, the plurality of analytes may be a clonal amplification colony. In other instances, the plurality of analytes may be different (e.g., comprise different sequences). In some examples, the plurality of analytes is immobilized to the individually addressable location via a support, such as a bead. In some examples, a bead comprises a plurality of amplification products, as analytes, immobilized thereto, and the bead is immobilized to an individually addressable location on the substrate. In another example, the bead is immobilized to an individually addressable location on the substrate and is configured to capture or bind to a plurality of analytes. In another example, a plurality of reagents is immobilized to an individually addressable location on the substrate via a support, such as a bead. The plurality of reagents may be configured for capturing or binding an analyte or another reagent. The plurality of reagents may be configured for release from the bead. The plurality of reagents bound to the bead may be releasable prior to, during, or subsequent to capturing or binding, or otherwise interacting with, an analyte or another reagent. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.).

### Generating Sequencing Data Using Flow Sequencing Methods

Sequencing data can be generated using a flow sequencing method that includes extending a primer hybridized to a template polynucleotide molecule according to a pre-determined flow cycle or flow order where, in any given flow position, a type of nucleotide base is accessible to the extending primer. More commonly, a single type of nucleotide base is used in any given sequencing flow, although in some variations, two or three different types of nucleotide bases may be used, which allows for a faster primer extension but may provide less sequencing data about the sequence region. At least some of the nucleotides of the particular base type can include a label, which upon incorporation of the labeled nucleotides into the extending primer renders a detectable signal. The resulting sequence by which such nucleotides are incorporated into the extended primer should be the reverse complement of the sequence of the template polynucleotide molecule. For example, sequencing data may be generated using a flow sequencing method that includes i) extending a primer using labeled nucleotides and ii) detecting the presence or absence of a labeled nucleotide incorporated into the extending primer. Flow sequencing methods may also be referred to as “natural sequencing-by-synthesis,” “mostly natural sequencing-by-synthesis,” or “non-terminated sequencing-by-synthesis” methods. Example methods are described in U.S. Pat. No. 8,772,473; published International application WO 2021/007495; published International application WO 2020/0227143; and published International application WO 2020/227137; each of which is incorporated herein by reference in its entirety. While the following description is provided in reference to flow sequencing methods, it is understood that other sequencing methods may be used to sequence all or a portion of the sequenced region.

Flow sequencing includes the use of nucleotides to extend the primer hybridized to the polynucleotide (e.g., to the template molecule). Nucleotides of a given base type (e.g., A, C, G, T, U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added), although two or three different types of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

The nucleotides can be introduced at a determined order during the course of primer extension, which may optionally be further divided into cycles. Nucleotides are added stepwise, which allows incorporation of the added nucleotide to the end of the sequencing primer of a complementary base in the template strand is present. The cycles may have the same order of nucleotides and number of different base types or a different order of nucleotides and/or a different number of different base types. Solely by way of example, the order of a first cycle may be A-T-G-C and the order of a second cycle may be A-T-C-G. In some instances, the order of any cycle may be any permutation of the nucleotides A, G, C, and T (or U). Between the introductions of different nucleotides, unincorporated nucleotides may be removed, for example by washing the sequencing platform with a wash fluid.

A polymerase can be used to extend a sequencing primer by incorporating one or more nucleotides at the end of the primer in a template-dependent manner. In some embodiments, the polymerase is a DNA polymerase. The polymerase may be a naturally occurring polymerase or a synthetic (e.g., mutant) polymerase. The polymerase can be added at an initial step of primer extension, although supplemental polymerase may optionally be added during sequencing, for example with the stepwise addition of nucleotides or after a number of flow cycles. Example polymerases include a DNA polymerase, an RNA polymerase, a thermostable polymerase, a wild-type polymerase, a modified polymerase, Bst DNA polymerase, Bst 2.0 DNA polymerase Bst 3.0 DNA polymerase, Bsu DNA polymerase, E. coli DNA polymerase I, T7 DNA polymerase, bacteriophage T4 DNA polymerase 029 (phi29) DNA polymerase, Taq polymerase, Tth polymerase, Tli polymerase, Pfu polymerase, and SeqAmp DNA polymerase.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template polynucleotide can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiment, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

The sequencing data can be generated by sequencing the test nucleic acid molecule using non-terminating nucleotides provided in separate nucleotide flows according to a flow-cycle order. The sequencing data can include flow signals at flow positions that each corresponds to a flow of a particular nucleotide. Using this uniquely structured data set, the nucleic acid molecule (or molecules) can be analyzed in “flowspace” rather than “basespace” (also referred to as “nucleotide space” or “sequence space”). The flowspace data depend on additional information related to the flow-cycle order, which is not carried by basespace data. See, for example, published International application WO 2020/227137.

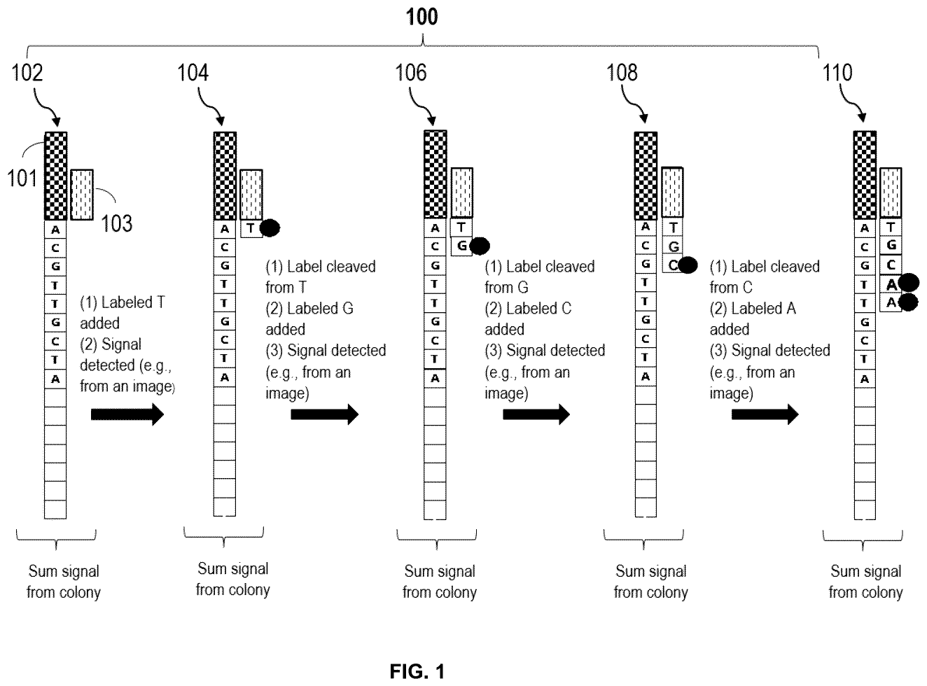

FIG. 1 illustrates an example flow sequencing method that can be used to generate the sequencing data described herein. In some embodiments, polynucleotides may be bound to a surface (e.g., the surface of a bead attached to a substrate), as described in detail herein. The polynucleotides can include a nucleic acid sequence of interest (also referred to as a “template sequence”) and can further include a sequencing adapter sequence. The nucleic acid sequence of interest can be a nucleic acid molecule from or derived from a sample of a subject.

In the depicted example of flow cycle 100 in FIG. 1, the polynucleotide includes an adaptor sequence 101 followed by the nucleic acid sequence of interest (e.g., “ACGTTGCTA . . . ”, or the “template polynucleotide”). The adapter sequence 101 can include a sequencing primer hybridization site. The adapter sequence 101 (hence, the polynucleotide) can be immobilized or deposited on a substrate. The substrate can be a bead. At step 102, a sequencing primer 103 is hybridized to the adapter sequence 101 of the polynucleotide at the sequencing primer hybridization site of the adapter sequence 101.

The sequencing primer is then extended in a series of flow cycles. In a flow cycle, the hybrid (i.e., the complex of the polynucleotide comprising the adapter sequence 101 hybridized to the sequencing primer) is combined with nucleotides (e.g., at least partially labeled nucleotides) and one or more signals indicating nucleotide incorporation into the sequencing primer may be detected. In the depicted example, the flow cycle 100 includes four flow steps 104, 106, 108, and 110. In a given flow step, a single type of nucleobase is combined with the hybrid according to the flow-cycle order T-G-C-A. As shown in FIG. 1, in flow step 104, labeled T nucleotides are combined with the hybrid (and can be incorporated into the growing strand); in flow step 106, labeled G nucleotides are combined with the hybrid (and can be incorporated into the growing strand); in flow step 108, labeled C nucleotides are combined with the hybrid (and can be incorporated into the growing strand); in flow step 110, labeled A nucleotides are combined with the hybrid (and can be incorporated into the growing strand). The flow-cycle order can vary. For example, the flow cycle order can be G-C-A-T, C-A-T-G, G-T-C-A, or other combinations of the sequential incorporations of nucleotides T, G, C, A (or other nucleotides).

At 104, labeled T nucleotides (the solid circle in FIG. 1 represents a label) are combined with the hybrid. Since the T base is complementary to the A base in the template polynucleotide, labeled T nucleotide is incorporated into the extending primer to form the hybrid as shown in 104. Further, a signal indicative of the incorporation of labeled T nucleotide into the sequencing primer (or extending primer) can be detected. The signal may be detected, for example, by imaging the surface the polynucleotides are deposited on (e.g., surface of beads of a sequencing platform) and analyzing the resulting image(s). In some embodiments, the sequencing platform may be washed with a wash buffer to remove unincorporated nucleotides prior to signal detection. In some embodiments, the detection of the signal is based on image processing techniques described herein.

At step 106, the label on the labeled T nucleotide may be removed from the incorporated T nucleotide (e.g., by cleaving the label from the nucleotide). The sequencing method can then be continued with the next base in the flow order, G in the example illustrated in FIG. 1. At step 106, labeled G nucleotides are combined with the hybrid. Since the G base is complementary to the C base in the template polynucleotide, labeled G nucleotide is incorporated to form the hybrid in 106. Further, a signal indicating the incorporation of the labeled G nucleotide into the sequencing primer (or extending primer) can be detected.

At step 108, the label on the labeled G nucleotide may be removed from the G nucleotide (e.g., by cleaving the label from the nucleotide). The sequencing method can then be continued with the next base in the flow order, C. At step 108, labeled C nucleotides are combined with the hybrid. Since the C base is complementary to the G base in the template polynucleotide, the labeled C nucleotide is incorporated into the extending primer to form the hybrid in 108. Further, a signal indicating the incorporation of the labeled C nucleotide into the sequencing primer (or extending primer) can be detected.

At step 110, the label on the labeled C nucleotide may be removed from the C nucleotide (e.g., by cleaving the label from the nucleotide). The sequencing method can then be continued with the next base in the flow order, A. At step 110, labeled A nucleotides are combined with the hybrid. Since the A base is complementary to the T base in the template polynucleotide, labeled A nucleotides are incorporated into the extending primer to form the hybrid in 110. Further, a signal indicating the incorporation of the labeled A nucleotide into the sequencing primer (or extending primer) can be detected. In step 110, because the template sequence includes two consecutive T bases, two A nucleotides are incorporated into the extending sequencing primer. Thus, the detected signal intensity indicating the incorporation of two A nucleotides may be greater than the signal intensity indicating the incorporation of a single nucleotide.

While each flow step in the example flow sequencing method in FIG. 1 results in incorporation of one or more nucleotides (and thus a detected signal indicating such incorporation), it should be appreciated that not all flow steps result in incorporation of nucleotides. In some flow steps, no nucleotide base may be incorporated (for example, in the absence of a complementary base in the template polynucleotide). For example, if C nucleotides are combined with a hybrid having a C base, no incorporation would occur and thus no signal indicative of an incorporation would be detected. Further, as shown in step 110, two nucleotides or more than two nucleotides may be incorporated into the sequencing primer for larger homopolymer lengths in the nucleic acid sequence of interest.

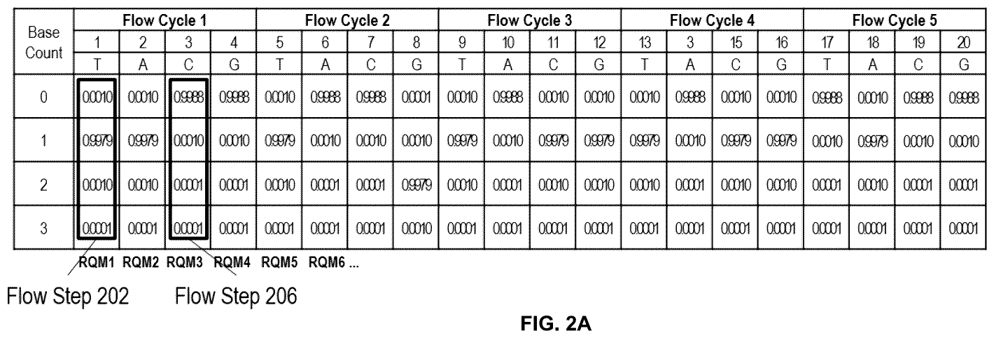

FIG. 2A illustrates an example summary of detected signals after five example flow cycles are performed, in accordance with some embodiments. Solely by way of example, a primer extended using a repeating flow-cycle order of T-A-C-G may result in a sequencing data flowgram set shown in FIG. 2A. Each column in FIG. 2A corresponds to a flow step and the values in each column collectively represent the detected signal intensity in the corresponding flow step, as described below.

In each flow step, the flow signal can be determined from an analog signal that is detected during the sequencing process, such as a fluorescent signal of the one or more bases incorporated into the sequencing primer during sequencing. Although an integer number of zero or more bases are incorporated at any given flow position, a given analog signal many not perfectly match with the analog signal. Therefore, in some embodiments, for a given flow step (e.g., flow step 202), the detected signal intensity can be expressed in probabilistic terms. Specifically, the detected signal intensity can be expressed in four likelihood values corresponding to 0 base, 1 base, 2 bases, and 3 bases, respectively.

In the depicted example, for flow step 202, the detected signal intensity is expressed by a first likelihood value of 0.001 for 0 base, a second likelihood value of 0.9979 for 1 base, a third likelihood value of 0.001 for 3 bases, and a fourth likelihood value of 0.0001 for 4 bases. This can be interpreted to indicate that there is a high statistical likelihood that one nucleotide base has been incorporated. In the depicted example, the incorporation is a T since the flow step introduced labeled T nucleotides, which means there is an A in the template.

On the other hand, in flow step 206, the detected signal intensity is expressed by a first likelihood value of 0.9988 for 0 base, a second likelihood value of 0.001 for 1 base, a third likelihood value of 0.001 for 3 bases, and a fourth likelihood value of 0.0001 for 4 bases. This can be interpreted to indicate that there is a high likelihood that no nucleotide base has been incorporated. In the depicted example, no C has been incorporated.

Accordingly, the flowgram set in FIG. 2A is formatted as a sparse matrix, with a flow signal represented by a plurality of likelihood values indicating a plurality of likelihoods for a plurality of base homopolymer length counts (e.g., 0 base count, 1 base count, 2 base counts, and 3 base counts) at each flow position.

The homopolymer length likelihood may vary, for example, based on the noise or other artifacts present during detection of the analog signal during sequencing. In some embodiments, if the homopolymer length likelihood statistical parameter or likelihood is below a predetermined threshold, the parameter may be set to a predetermined non-zero value that is substantially zero (i.e., some very small value or negligible value) to aid the downstream statistical analysis further discussed herein, wherein a true zero value may give rise to a computational error or insufficiently differentiate between levels of unlikelihood, e.g., very unlikely (0.0001) and inconceivable (0).

With reference to FIG. 2B, a preliminary sequence can be determined based on the flowgram in FIG. 2A. For example, the most likely sequence can be determined by selecting the base count with the highest likelihood at each flow position, as shown by the stars in FIG. 2B. Thus, the preliminary sequence 210 can be determined as: TATGGTCGTCGA (SEQ ID NO: 1257). From the preliminary sequence (e.g., preliminary sequence 210), the reverse complement (i.e., the template strand or the nucleic acid sequence of interest) can be readily determined. Further, the likelihood of this sequencing data set, given the TATGGTCGTCGA (SEQ ID NO: 1257) sequence (or the reverse complement), can be determined as the product of the selected likelihood at each flow position.

The signal for any flow position in the sequencing data is flow-order-dependent in that the flow order used to sequence the polynucleotide at any base position can affect the flow signal at that position. Random fragmentation of nucleic acid molecules (either in vivo fragmentation, such as cell-free DNA, or in vitro fragmentation, such as by sonication or enzymatic digestion) that overlap at the same locus results in multiple different sequencing start sites (relative to the locus) for the nucleic acid molecules.

Sequencing data, such as a flowgram, is based on the detection of a signal detected from an incorporated nucleotide and the order of nucleotide introduction. Take, for example, the flowing template sequences: CTG and CAG, and a repeating flow cycle of T-A-C-G (that is, sequential addition of T, A, C, and G nucleotides, each of which would be incorporated into the primer only if a complementary base is present in the template polynucleotide). A resulting example flowgram is shown in Table 1, where 1 indicates incorporation of an introduced nucleotide and 0 indicates no incorporation of an introduced nucleotide. The flowgram can be used to determine the sequence of the template strand.

TABLE 1
Examples of flowgrams (e.g., vector signal
information for nucleic acid sequences)

| | |
|---|---|
| Cycle 1 | Cycle 2 |

| | | | | | | | | |
|---|---|---|---|---|---|---|---|---|
| Flow: | 0 | 1 | 2 | 3 | 4 | 5 | 6 | 7 |
| Sequence | T | A | C | G | T | A | C | G |
| CTG | 0 | 0 | 0 | 1 | 0 | 1 | 1 | 0 |
| CAG | 0 | 0 | 0 | 1 | 1 | 0 | 1 | 0 |
| CCG | 0 | 0 | 0 | 2 | 0 | 0 | 1 | 0 |

The flowgram can be used to quantitatively determine a number of incorporated nucleotides from each stepwise introduction (e.g., for each nucleotide in a cycle). For example, a sequence of CCG would first incorporate two G bases, and any signal emitted by the labeled two bases would have a greater intensity as compared with the incorporation of a single base. This is shown in Table 1 (e.g., the 2 value in the third row). The flowgram of Table 1 indicates the presence or absence of each indicated base, but flowgrams can also provide additional information including the number of bases incorporated at the given step.

Prior to generating the sequencing data, the polynucleotide is hybridized at a hybridization site to a sequencing primer to generate a hybridized template. The polynucleotide may be ligated to an adapter during sequencing library preparation, such as during the attachment of one or more barcode regions. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support and/or substrate) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. Pat. No. 10,344,328 and international patent application WO 2020/227143, each of which is incorporated herein by reference in its entirety.

The primer hybridized to the polynucleotide is extended through the nucleic acid molecule using the separate nucleotide flows according to the flow order (which may be cyclical according to a flow-cycle order), and incorporation of a nucleotide can be detected as described above, thereby generating the sequencing data set (via a flowgram) for the nucleic acid molecule.

Primer extension using flow sequencing allows for long-range sequencing on the order of hundreds or even thousands of bases in length. The number of flow steps or cycles can be increased or decreased to obtain the desired sequencing length. Extension of the primer can include one or more flow steps for stepwise extension of the primer using nucleotides having one or more different base types. In some embodiments, extension of the primer includes between 1 and about 1000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, or between about 500 and about 1000 flow steps. The flow steps may be segmented into identical or different flow cycles. The number of bases incorporated into the primer depends on the sequence of the sequenced region, and the flow order used to extend the primer. In some embodiments, the sequenced region is about 1 base to about 4000 bases in length, such as about 1 base to about 10 bases in length, about 10 bases to about 20 bases in length, about 20 bases to about 50 bases in length, about 50 bases to about 100 bases in length, about 100 bases to about 250 bases in length, about 250 bases to about 500 bases in length, about 500 bases to about 1000 bases in length, about 1000 bases to about 2000 bases in length, or about 2000 bases to about 4000 bases in length.

The polynucleotides used in the methods described herein may be obtained from any suitable biological source, for example a tissue sample, a blood sample, a plasma sample, a saliva sample, a fecal sample, or a urine sample. The polynucleotides may be DNA or RNA polynucleotides. In some embodiments, RNA polynucleotides are reverse transcribed into DNA polynucleotides prior to hybridizing the polynucleotide to the sequencing primer. In some embodiments, the polynucleotide is a cell-free DNA (cfDNA), such as a circulating tumor DNA (ctDNA) or a fetal cell-free DNA. The nucleic acid molecules may be randomly fragmented, for example in vivo (e.g., as in cfDNA) or in vitro (for example, by sonication or enzymatic fragmentation).

Libraries of the polynucleotides may be prepared through known methods. In some embodiments, the polynucleotides may be ligated to an adapter sequence. The adapter sequence may include a hybridization sequence that hybridized to the primer extended during the generated of the coupled sequencing read pair.

In some embodiments, the sequencing data is obtained without amplifying the nucleic acid molecules prior to establishing sequencing colonies (also referred to as sequencing clusters). Methods for generating sequencing colonies include bridge amplification or emulsion PCR Methods that rely on shotgun sequencing and calling a consensus sequence generally label nucleic acid molecules using unique molecular identifiers (UMIs) and amplify the nucleic acid molecules to generate numerous copies of the same nucleic acid molecules that are independently sequenced. The amplified nucleic acid molecules can then be attached to a surface and bridge amplified to generate sequencing clusters that are independently sequenced. The UMIs can then be used to associate the independently sequenced nucleic acid molecules. However, the amplification process can introduce errors into the nucleic acid molecules, for example due to the limited fidelity of the DNA polymerase. In some embodiments, the nucleic acid molecules are not amplified prior to amplification to generate colonies for obtaining sequencing data. In some embodiments, the nucleic acid sequencing data is obtained without the use of unique molecular identifiers (UMIs).

### Barcode Selection

Provided herein are methods, systems, compositions, and kits for generating or selecting a set of barcode sequences. Sets of barcode sequences may be selected from a plurality of possible barcode sequences based on one or more selection criteria, including, but not limited to: barcode sequence length, distinguishability from all other barcode sequences within the plurality of barcode sequences, number of flow cycles (as described above) to sequence the barcode sequence, etc. One or more methods described herein may comprise a computer-implemented method, and one or more processes of a method may be performed using at least one processor. Such a method (e.g., computer-implemented method) may comprise providing a plurality of barcode sequences and generating a plurality of matrices of flow data, in which each matrix of the plurality of matrices corresponds to a different barcode sequence of the plurality of barcode sequences. Each matrix of flow data may comprise information, such as sequencing information obtained from the methods and processes described herein.

For example, each matrix of flow data may comprise sequence data generated from a plurality of flow cycles, which flow data may be representative of nucleotide addition events for a given barcode sequence. The method may further comprise applying one or more constraints on the plurality of matrices of flow data to generate a first set of filtered matrices, filtering the first set of filtered matrices using a first criterion to generate a second set of filtered matrices, and filtering the second set of filtered matrices based on a second criterion to generate a third set of filtered matrices. Each matrix of the third set of filtered matrices may correspond to a barcode sequence of the plurality of barcode sequences. In some instances, the third set of filtered matrices corresponds to a subset of barcode sequences of the plurality of barcode sequences and may be electronically output. The set of barcode sequences generated from such a method may be useful in generating sets of sufficiently diverse barcode sequences that satisfy one or more selection criteria.

The plurality of matrices of flow data may be generated empirically (e.g., in vitro) or computationally (e.g., in silico). In some instances, the plurality of matrices of flow data may be generated using at least one processor and may comprise use of a simulation or algorithm to prepare the flow data. In other instances, the plurality of matrices of flow data may generated empirically, e.g., by performing the method as described with respect to FIG. 1. For a given barcode sequence, the flow data may comprise information on the number of flow cycles (e.g., the number of iterations of flow cycles) as well as the number of nucleotides added per flow cycle.

Advantageously, the set of barcode sequences that are generated or selected according to the methods, systems, compositions, and kits described herein may be used as reagents, or as reagent components, in the sequencing systems and methods described herein. The set of barcode sequences may be particularly useful for distinguishing between any two barcoded analytes (e.g., a bead comprising a nucleic acid analyte, which nucleic acid analyte has been barcoded such as to contain a barcode sequence or a complement thereof, of the set of barcode sequences) that are immobilized on a planar substrate, even if such barcoded analytes are immobilized at relatively high density (e.g., on the order of 1 million, 10 million, 100 million, 1 billion, 10 billion, 100 billion, or more beads immobilized in a substrate having a maximum surface diameter of at most 20 inches (˜50.8 cm)).

In an example, a plurality of barcode sequences (e.g., single-stranded molecules or partially single-stranded molecules comprising an annealed primer) comprising different sequences may be provided on a substrate, as is described elsewhere herein. The method of sequencing by synthesis (e.g., as illustrated by FIG. 1) may be performed, in which a first nucleotide base or analog is added to the substrate (e.g., a thymine or analog thereof), and the substrate is subjected to conditions to allow the first nucleotide base to incorporate into any barcode sequence comprising a complementary base (e.g., an adenine or analog thereof). Detection may be performed across the substrate to generate a signal, for each barcode sequence, which is indicative of a nucleotide addition or incorporation event. In some instances, the signal (or lack thereof) generated from the detection operation may be registered, e.g., using at least one processor, to each of the barcode sequences. For example, a first flow cycle may be performed in which thymine is added, and barcode sequences comprising an adenine at a first location (e.g., a single-stranded portion adjacent to a double-stranded region or primer-annealed region) along the barcode sequence may incorporate the thymine(s), which may be registered, using the at least one processor, as a “1”, “2”, “3”, etc., depending on the number of adjacent adenines in the barcode sequence. Barcode sequences that do not have an adenine at the first location may be registered as “0”. Subsequently, a second flow cycle may be performed in which guanine is added, and barcode sequences comprising a cytosine at a second location (e.g., a single-stranded portion adjacent to the first location) may incorporate the guanine(s), and the number of incorporated guanines may be registered for each barcode sequence. A third flow cycle may be performed in which cytosine is added, and a fourth flow cycle may be performed in which adenine is added. In such an example, in which the flow sequence (e.g., comprising four flow cycles) is iteratively T-G-C-A, a barcode sequence comprising a sequence of TGCATT may have registered flow cycle values as 1, 1, 1, 1, 2, representative of 1 nucleotide addition of T, one nucleotide addition of G, one nucleotide addition of C, one nucleotide addition of A, and 2 nucleotide additions of T in accordance with nucleotides introduced during the flow sequence. However, a different barcode sequence comprising a sequence of TGCAC may have the registered flow cycle values as 1, 1, 1, 1, 0, 0, representative of 1 nucleotide addition of T, one nucleotide addition of G, one nucleotide addition of C, one nucleotide addition of A, zero nucleotide additions of T, and zero nucleotide additions of G. Additional examples of expected flow cycle values can be found in Examples 1 and 2 below. It can be appreciated that the order of nucleotide base addition (e.g., the flow sequence T, G, C, A) is for illustrative purposes only, and that any order and N-mer (e.g., monomer, dimer, trimer, etc.) of nucleotide bases may be added for each flow cycle.

Barcode sequences typically begin with a preamble sequence, which is determined based on the flow sequence to be used. For example, when the desired flow cycle sequence is T, G, C, A, the preamble sequence can be T, G, C, A, thereby providing flow cycle analog signal values of 1, 1, 1, 1. In some instances, such a preamble sequence is of use for identifying sequencing colonies during signal detection and/or in providing a baseline signal level for downstream analog signal analysis. In some instances, all barcode sequences after the preamble sequence may start with a single nucleotide of a same type. For example, in all instances, all barcodes after the constant preamble sequence may start with a single A, a single T (or a U), a single C. or a single G. In some instances, all barcodes end with a constant sequence to support un-biased library prep. In some instances, the constant sequence is GAT. In some instances, the constant sequence is any series of three nucleotides. In some instances, the constant sequence is a series of more than 3 nucleotides (e.g., 4 or more nucleotides, 5 or more nucleotides, etc.).

The flow cycle values for each barcode sequence may be input, e.g., using the at least one processor, into a matrix or structure of flow data, such that each barcode sequence comprises a matrix or structure of flow data. Each matrix or structure may comprise a plurality of elements indicative of the flow cycle values for each flow cycle. For example, continuing with the abovementioned example of a iterative set of flow cycles of adding T-G-C-A, a 5-round flow cycle adds the nucleotides in a T-G-C-A-T order, and a barcode sequence of TGCATT results in a matrix or structure comprising the elements (e.g., flow cycle values) of 1, 1, 1, 1, 2. In some instances, the matrix or structure of flow data for each barcode sequence comprises a 1×N or an N×1 vector, in which N is the number of flow cycles. For example, for a flow sequence of T-G-C-A-T, five rounds of flow cycles are performed, N=5, and the matrix of flow data may comprise a 1×5 vector (or a 5×1 vector).

The individual flow cycle values may be referred to herein as H-mers, in which H indicates the magnitude of the flow cycle value (e.g., 0, 1, 2, etc.) and the corresponding number of incorporated nucleotides for each flow cycle performed. For example, for a flow cycle resulting in a single nucleotide addition, H=1. For double nucleotide addition events (e.g., TT, GG, CC, AA), H=2, and for triple nucleotide addition events (e.g., TIT, GGG, CCC, AAA), H=3, and so on. For events in which the nucleotide in the flow sequence is not added, H=0. Accordingly, the matrix of flow data may comprise a 1×N vector, in which each element (e.g., flow cycle value) of the 1×N vector is an H-mer (e.g., a vector comprising N elements, each element of which is an H-mer). As such, for a given flow sequence (e.g., iterative T-G-C-A), a given vector (or matrix or structure) may inform the number of nucleotides added per flow cycle, and thus the sequence of the corresponding barcode sequence may be determined.

The plurality of matrices of flow data may be subjected to filtering or application of one or more constraints to generate a first set of filtered matrices. For example, for a given set of barcode sequences (e.g., a set of possible barcode sequences), each barcode sequence of the given set may comprise a matrix of flow data. Subsequent to filtering or application of one or more constraints, one or more matrices of flow data may be removed. As each matrix of flow data corresponds to a single barcode sequence, the filtering or application of one or more constraints may result in removal of barcode sequences from the given set of barcode sequences. Non-limiting examples of constraints include: a minimum, maximum, or range of one or more parameters, e.g., number of elements or flow cycles, H-mer magnitude (e.g., value of H) for each element in the matrix (or vector), number of H-mers above a threshold H value (e.g., H=7). For example, in some instances, it may be useful to generate a set of barcode sequences that can be sequenced within a certain number of flow cycles, e.g., to minimize reagent waste. Using iterative T-G-C-A flow cycles as an example, and an example barcode sequence of ACACG, the resultant matrix of flow data comprises 14 elements (flow cycle values of 0, 0, 0, 1, 0, 0, 1, 1, 0, 0, 1, 0, 0, 1) before the entire 5-base pair barcode sequence is uncovered or sequenced. In contrast, an example barcode sequence of TGCATT results in a matrix of flow data comprising 5 elements (flow cycle values of 1, 1, 1, 1, 2), which reduces the number of total flow cycles and results in reduced reagent waste. As such, it may be beneficial to filter the matrices of flow data to a predetermined constraint (e.g., a maximum number of flow cycles that are required to sequence the entire barcode sequence). In another example, it may be useful or beneficial to apply one or more constraints on H-mer magnitude. For example, in some instances, it may be challenging (e.g., computationally demanding) to distinguish the signal indicative of a 7-mer in comparison to an 8-mer (e.g., TTTTTTT compared to TTTTTTTT), and a maximum H-mer constraint may be useful for ease of signal analysis. In other examples, it may be useful or beneficial to apply a constraint of a maximum number of H-mers (e.g., no more than five 4-mers in any one barcode sequence, no more than two 6-mers in any one barcode sequence, etc.). The resultant first set of filtered matrices may comprise barcode sequences that have been selected to fulfill the one or more applied constraints.

The first set of filtered matrices may be subjected to further filtration processes. The first set of filtered matrices may be subjected to any number of filtration processes to generate a further filtered matrix (e.g., a second set of filtered matrices). In some instances, the first set of filtered matrices are filtered using a first criterion, e.g., a barcode sequence length (e.g., number of nucleotides). For example, it may be useful to generate a set of barcode sequences that are uniform in length, and the first set of filtered matrices may be filtered for barcodes sequences that have a particular length (e.g., barcode sequences comprising at least 5 base pairs, 6 base pairs, 7 base pairs, 8 base pairs, 9 base pairs, 10 base pairs, 11 base pairs, 12 base pairs, 13 base pairs, 14 base pairs, 15 base pairs, 16 base pairs, 17 base pairs, 18 base pairs, 19 base pairs, 20 base pairs, 21 base pairs, 22 base pairs, 23 base pairs, 24 base pairs, 25 base pairs, 26 base pairs, 27 base pairs, 28 base pairs, 29 base pairs, 30 base pairs, or greater) or a range of lengths (e.g., a barcode sequence having from 9 to 11 base pairs). Examples of the range of lengths can be from 9 to 30 base pairs, from 9 to 25 base pairs, from 9 to 20 base pairs, from 9 to 18 base pairs, from 9 to 16 base pairs, from 9 to 15 base pairs, from 9 to 14 base pairs, from 9 to 13 base pairs, or from 9 to 12 base pairs, or other ranges. Further examples of barcode sequences are barcode sequences comprising 5 base pairs, 6 base pairs, 7 base pairs, 8 base pairs, 9 base pairs, 10 base pairs, 11 base pairs, 12 base pairs, 13 base pairs, 14 base pairs, 15 base pairs, 16 base pairs, 17 base pairs, 18 base pairs, 19 base pairs, 20 base pairs, 21 base pairs, 22 base pairs, 23 base pairs, 24 base pairs, 25 base pairs, 26 base pairs, 27 base pairs, 28 base pairs, 29 base pairs, 30 base pairs, or greater. In some examples, it may be useful to generate a set of barcode sequences that have a maximum or minimum length, and the first set of filtered matrices may be filtered for barcode sequences that have the maximum or minimum length.

In some instances, the second set of filtered matrices may be subjected to additional filtering (e.g., using a second criterion) to generate a third set of filtered matrices. In some instances, the second criterion may comprise an edit distance between matrices in the second set of filtered matrices. In such cases, the additional filtering may comprise calculating (e.g., using the at least one processor) an edit distance for all pairs of matrices and removing matrices that do not fall within a set threshold or range of edit distances. The edit distance may be calculated using a variety of approaches. In some instances, the edit distance can be calculated by counting (e.g., using the at least one processor), a number of different elements between two matrices of the second set of filtered matrices. The edit distance may be any useful edit distance (e.g., a Levenshtein distance, a longest common subsequence distance, a Hamming distance, a Jardo distance, a Damerau-Levenshtein distance, or analogs or derivatives thereof).

As one example, a Hamming distance may be calculated for all pairs of matrices within the set (e.g., second set of filtered matrices). In such an example, for any given pair of matrices, each position (e.g., element, which may comprise a flow cycle value or H-mer) of the first matrix of the pair is compared to the corresponding position in the second matrix of the pair. If the values differ for a given position, a value of 1 distance unit is added (e.g., every position in the pair of matrices that differs increases the value of the edit distance between the pair of matrices by 1). By way of example, a first matrix comprising a 1×5 vector of [0, 0, 1, 1, 2] and a second matrix comprising a 1×5 vector of [0, 0, 3, 2, 2] has an edit distance of 2, as two positions (the third and fourth elements) within the matrices differ in value. Each position in the pair of matrices that do not differ in value (e.g., the first, second, and fifth elements in this example) does not increase the edit distance.

The edit distance threshold between all pairs of matrices (e.g., in the second set of filtered matrices) may be set at any useful value. In some instances, a higher edit distance threshold may be applied in order to increase the distinction between barcode sequences (e.g., to increase the difference between barcode sequences, thus decreasing the complexity of downstream analysis). The edit distance threshold may be at least 1, at least 2, at least 3, at least 4, at least 5, at least 6, at least 7, at least 8, at least 9, at least 10 distance units, or more. In other instances, a maximum edit distance threshold may be set, e.g., at most 10, at most 9, at most 8, at most 7, at most 6, at most 5, at most 4, at most 3, at most 2, or at most 1 distance units.

The third set of filtered matrices may correspond to barcode sequences that meet a plurality of criteria (e.g., sequence length, number of flows, edit distance threshold, etc.). It can be appreciated that while various filtering and constraint application examples are provided herein, the order or number of filtering or constraint application events may be altered. For example, the first set of filtered matrices may be filtered for edit distance prior to filtering for barcode sequence length. Similarly, the applied constraints may be performed subsequent to the one or more filtering operations. Any number and combination of filtering or constraint application events may be performed, e.g., 3 events, 4, events, 5 events, 6 events, 7 events, 8 events, 9 events, 10 events, or more. In some instances, a maximum number of filter or constraint application events may be performed, e.g., at most about 10 events, at most 9 events, at most 8 events, at most 7 events, at most 6 events, at most 5 events, at most 4 events, at most 3 events, at most 2 events, etc.

As further described in Examples 1 and 2 below, the methods described herein may be beneficial in generating sufficiently diverse barcode sequences that satisfy one or more applied constraints or filters. Beneficially, barcode sequences may be useful in analyzing or characterizing analytes (e.g., proteins, nucleic acid molecules, etc.), e.g., by uniquely identifying or labeling the analytes from arising from a particular origin, partition, sample, etc. The methods described herein may be useful, for example, in whole genome sequencing or targeted sequencing. In some instances, the barcode sequences may be used for barcoding of analytes (e.g., nucleic acid molecules) and analyzed (e.g., via sequencing) without prior indexing.

In another aspect of the present disclosure, provided herein are systems, compositions, and kits. A composition or system of the present disclosure may comprise a non-naturally occurring nucleic acid barcode molecule comprising a sequence of any one of SEQ ID NOs: 1-1256. In some instances, the non-naturally occurring nucleic acid barcode molecule may be coupled to a support, e.g., a bead. The support may comprise any number or combination of the sequences disclosed herein (e.g., SEQ ID NOs: 1-1256). In some instances, the support may comprise any number or combination of the sequences SEQ ID NOs: 1-238. In some instances, the support may comprise any number of combination of the sequences SEQ ID NOs: 239-1256. In some instances, the support may comprise any number or combination of sequences, where each sequence requires a same number of flows to be fully sequenced.

Also provided herein is a kit comprising a non-naturally occurring nucleic acid barcode molecule comprising a sequence of any one of SEQ ID NOs: 1-1256 and instructions for using the non-naturally occurring nucleic acid barcode molecule. In some instances, a kit comprises at least 8, 16, 24, 48, 96 non-naturally occurring nucleic acid barcode molecules, where each barcode molecule comprises a different sequence selected from the group consisting of SEQ ID NOs: 1-238. In some instances, a kit comprises at least 8, 16, 24, 48, 96 non-naturally occurring nucleic acid barcode molecules, where each barcode molecule comprises a different sequence selected from the group consisting of SEQ ID NOs: 239-1256.

Also provided herein is a composition, comprising a non-naturally occurring nucleic acid barcode molecule consisting of 10-30 linked nucleosides and having a sequence comprising at least 8 contiguous nucleosides (e.g., nucleotide base types) selected from (e.g., selected from a sequence within) the group consisting of SEQ ID NOs: 1-1256. In some instances, the composition comprises a non-naturally occurring nucleic acid barcode molecule consisting of 10-30 linked nucleosides and having a sequence comprising at least 8 contiguous nucleosides (e.g., nucleotide base types) selected from (e.g., selected from a sequence within) the group consisting of SEQ ID NOs: 1-238. In some instances, the composition comprises a non-naturally occurring nucleic acid barcode molecule consisting of 10-30 linked nucleosides and having a sequence comprising at least 8 contiguous nucleosides (e.g., nucleotide base types) selected from (e.g., selected from a sequence within) the group consisting of SEQ ID NOs: 239-1256. In some instances, the non-naturally occurring nucleic acid barcode molecule consists of 10, 11, 12, 13, 14, 15, 16, 17, 18, 19, 20, 21, 22, 23, 24, 25, 26, 27, 28, 29, or 30 contiguous nucleosides, or any range therein. In some instances, the sequence comprises at least 8, 9, 10, 11, 12, 13, 14, 15, 16, 17, 18, 19, 20, 21, 22, 23, 24, 25, 26, 27, 29, or 30 contiguous nucleosides selected from a sequence within the group consisting of SEQ ID NOs: 1-1256.

### Computer Systems

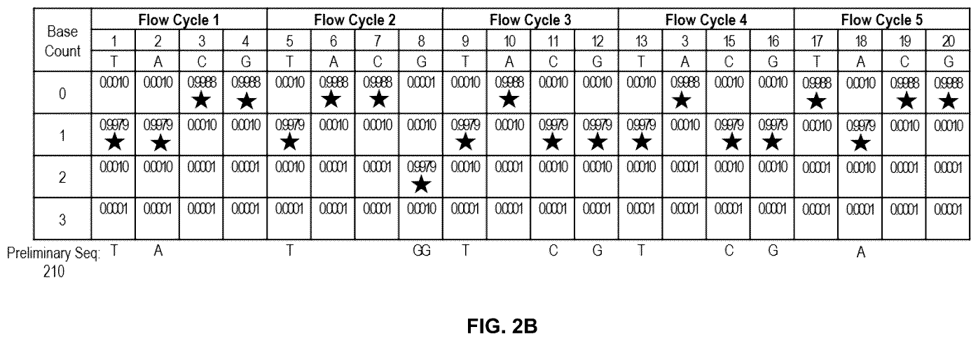

The present disclosure provides computer control systems that are programmed to implement methods of the disclosure. FIG. 3 shows a computer system 301 that is programmed or otherwise configured to implement methods of the disclosure, such as to control the systems described herein (e.g., reagent dispensing, detecting, etc.) and collect, receive, and/or analyze sequencing information. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine-readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example a map of analyte sequences and/or map of geolocation beads. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, spatially resolve a plurality of analyte sequences using sequencing information. The results of sequencing a plurality of nucleic acid molecules, optionally comprising barcode sequences, may be output, e.g., using a processor, as information in flow space (e.g., a matrix or vector of flow data), which may then be further processed.

### EXAMPLES

### Example 1—Generation and Selection of Barcode Sequences

As described herein, barcode sequences may be generated and selected (e.g., at one or more processors in computer system 301) based on one or more criteria and by performing one or more filtering processes. With regards to flow sequencing applications, these barcodes may be used to identify flows of interest from analog data (e.g., just from signals—such as optical signals—generated during sequencing, see, e.g., FIG. 1), instead of after sequencing (e.g., after basecalling).

The time-consuming process of identifying ˜100 million training reads in a substrate comprising 4 billion or more sequence reads may be avoided by identifying the training reads during signal collection (e.g., during sequencing by synthesis using detection of identifiable signals during each flow cycle). During signal collection, a sample data set, used for training may be copied to the monitoring computer system. Beneficially, instead of selecting the sample set randomly or after a nucleic acid base sequence is determined, the training set may be identified at flow 4 (e.g., in flow space) through the design of distinguishable barcode sequences.

The flow sequence used in this example is TGCA. In some instances, as described elsewhere herein, the flow sequence may be any other permutation of the nucleotides T or U, G, C, and A (e.g., GTAC, ACTG, etc.). In some instances, for example for non-WGS runs, a spike-in training data set may be added and used for training a model to evaluate the sample, non-WGS data. That training set may be labeled as described below in Table 2 to prevent contamination at the analysis level with the other, sample data. The training data set may comprise: a set of ˜100 million reads, comprising ˜80 million standard human reads and ˜20 million E. coli reads.

The training and sample data share one flow cycle sequence preamble (e.g., one iteration of T, G, C, A flows). The training data may be identified by a training data indication sequence that can be identified within one flow (e.g., a flow comprising one nucleotide base type). In some instances, the training data indication sequence is TT (e.g., a sequence that results in a double addition of a nucleotide). The analog signal detected from the incorporation of two nucleotides (e.g., a homopolymer of length 2) can be used to clearly discriminate reads that have the TT identification sequence from reads that lack the TT identification sequence. PP-22,n

TABLE 2
Training and sample identification sequences, showing
the comparison between basespace and flowspace.

| | Cycle 1 | | | | Cycle 2 | | | |
|---|---|---|---|---|---|---|---|---|
| Flows: | 0 | 1 | 2 | 3 | 4 | 5 | 6 | 7 |
| Sequence | T | G | C | A | T | G | C | A |
| Training data ID: T, G, C, A, T, | 1 | 1 | 1 | 1 | 2 | 0 | 0 | — |
| T . . . | | | | | | | | |
| Sample sequence ID: T, G, C, A, | 1 | 1 | 1 | 1 | 0 | 0 | 1 | — |
| C . . . | | | | | | | | |

Here in Table 2, flows 0-3 are the preamble (e.g., T, G, C, A, where the indexing begins at 0). Flow 4 (e.g., the first flow of the second flow cycle) identifies the double TT analog signal for training data reads. As shown in Table 2, the sample sequences have a different sequence ID (e.g., the first nucleotide base after the preamble sequence is a C instead of a double T. This may result in a flowgram for the second flow cycle of 0, 0, 1 . . . for all sample reads, as compared with the flowgram 2, 0, 0 . . . for all training data in the second flow cycle. In this way, contamination of training data may be prevented, thereby improving model training (e.g., by providing improved input data). Training data may be identified by a distinct signal at flow 4, where the signal output for training data is 2 and the signal output for sample data are 0. The strong analog signal separation between 2-mers and 0-mers prevents most mis-identifications. Further, confirmation of sample data identity can also include examination of flows 5 and 6, which are always 0, 1 for sample data sequencing reads and 0, 0 for training data sequencing reads.

In this example, a minimum number of barcodes were required (e.g., at least 96×2 different barcodes). Barcode sequences were thus determined for an effective length of 20 flows. The barcode sequences included the following regions: preamble (4 flows, 4 bases), constant prefix (3 flows 1 base), variable sequence, and constant post sequence (4 flows, 3 bases). Barcodes were kept at a constant length in flow space (e.g., each barcode can be fully sequenced in the same number of flows and requires the same number of flows to be fully sequenced). Barcodes were required to be an edit distance of at least 2 from each other barcode sequence (e.g., as measured in the vector space representing flow signals). In addition, each of the values in flow space were 0 or 1 (e.g., there are no homopolymers in base space greater than 1 in any of the barcode sequences). All barcodes in this set start with a single C (e.g., denoting sample data, as described above with respect to Table 2).

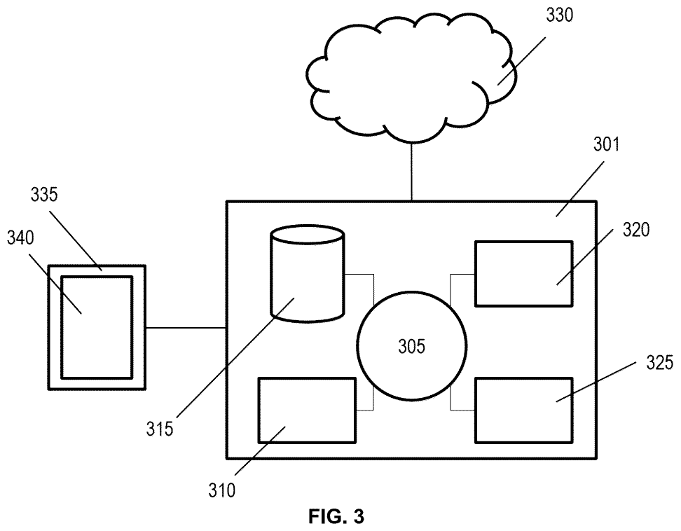

With the above-described restrictions, 20 flows were used to arrive at a set of 238 barcodes. Of these 11 flows are constant (e.g., 4 flows for the preamble, 3 flows constant prefix—the sample sequence ID, and 4 flows at the end of the barcode sequence), thereby leaving 9 flows (e.g., the variable sequence) as variable. In such an instance, these barcode variable sequences may have either 9 or 11 bases (e.g., there is variable length in base space). FIG. 4 illustrates a histogram of the number of base pairs in this set of barcodes. Table 3A lists SEQ ID NOs for the 238 barcode sequences.

TABLE 3A
List of example barcode sequences.

| SEQ ID NO: | Barcode |
|---|---|
| 1 | TGCACGTCATGAT |
| 2 | TGCACGTGATGAT |
| 3 | TGCACGTGCTGAT |
| 4 | TGCACGTGCAGAT |
| 5 | TGCACGACATGAT |
| 6 | TGCACGAGATGAT |
| 7 | TGCACGAGCTGAT |
| 8 | TGCACGAGCAGAT |
| 9 | TGCACGATATGAT |
| 10 | TGCACGATCTGAT |
| 11 | TGCACGATCAGAT |
| 12 | TGCACGATGTGAT |
| 13 | TGCACGATGAGAT |
| 14 | TGCACGATGCGAT |
| 15 | TGCACGATGCATGAT |
| 16 | TGCACGCGATGAT |
| 17 | TGCACGCGCTGAT |
| 18 | TGCACGCGCAGAT |
| 19 | TGCACGCTATGAT |
| 20 | TGCACGCTCTGAT |
| 21 | TGCACGCTCAGAT |
| 22 | TGCACGCTGTGAT |
| 23 | TGCACGCTGAGAT |
| 24 | TGCACGCTGCGAT |
| 25 | TGCACGCTGCATGAT |
| 26 | TGCACGCACTGAT |
| 27 | TGCACGCACAGAT |
| 28 | TGCACGCAGTGAT |
| 29 | TGCACGCAGAGAT |
| 30 | TGCACGCAGCGAT |
| 31 | TGCACGCAGCATGAT |
| 32 | TGCACGCATAGAT |
| 33 | TGCACGCATCGAT |
| 34 | TGCACGCATCATGAT |
| 35 | TGCACGCATGATGAT |
| 36 | TGCACGCATGCTGAT |
| 37 | TGCACGCATGCAGAT |
| 38 | TGCACTACATGAT |
| 39 | TGCACTAGATGAT |
| 40 | TGCACTAGCTGAT |
| 41 | TGCACTAGCAGAT |
| 42 | TGCACTATATGAT |
| 43 | TGCACTATCTGAT |
| 44 | TGCACTATCAGAT |
| 45 | TGCACTATGTGAT |
| 46 | TGCACTATGAGAT |
| 47 | TGCACTATGCGAT |
| 48 | TGCACTATGCATGAT |
| 49 | TGCACTCGATGAT |
| 50 | TGCACTCGCTGAT |
| 51 | TGCACTCGCAGAT |
| 52 | TGCACTCTATGAT |
| 53 | TGCACTCTCTGAT |
| 54 | TGCACTCTCAGAT |
| 55 | TGCACTCTGTGAT |
| 56 | TGCACTCTGAGAT |
| 57 | TGCACTCTGCGAT |
| 58 | TGCACTCTGCATGAT |
| 59 | TGCACTCACTGAT |
| 60 | TGCACTCACAGAT |
| 61 | TGCACTCAGTGAT |
| 62 | TGCACTCAGAGAT |
| 63 | TGCACTCAGCGAT |
| 64 | TGCACTCAGCATGAT |
| 65 | TGCACTCATAGAT |
| 66 | TGCACTCATCGAT |
| 67 | TGCACTCATCATGAT |
| 68 | TGCACTCATGATGAT |
| 69 | TGCACTCATGCTGAT |
| 70 | TGCACTCATGCAGAT |
| 71 | TGCACTGTATGAT |
| 72 | TGCACTGTCTGAT |
| 73 | TGCACTGTCAGAT |
| 74 | TGCACTGTGTGAT |
| 75 | TGCACTGTGAGAT |
| 76 | TGCACTGTGCGAT |
| 77 | TGCACTGTGCATGAT |
| 78 | TGCACTGACTGAT |
| 79 | TGCACTGACAGAT |
| 80 | TGCACTGAGTGAT |
| 81 | TGCACTGAGAGAT |
| 82 | TGCACTGAGCGAT |
| 83 | TGCACTGAGCATGAT |
| 84 | TGCACTGATAGAT |
| 85 | TGCACTGATCGAT |
| 86 | TGCACTGATCATGAT |
| 87 | TGCACTGATGATGAT |
| 88 | TGCACTGATGCTGAT |
| 89 | TGCACTGATGCAGAT |
| 90 | TGCACTGCGTGAT |
| 91 | TGCACTGCGAGAT |
| 92 | TGCACTGCGCGAT |
| 93 | TGCACTGCGCATGAT |
| 94 | TGCACTGCTAGAT |
| 95 | TGCACTGCTCGAT |
| 96 | TGCACTGCTCATGAT |
| 97 | TGCACTGCTGATGAT |
| 98 | TGCACTGCTGCTGAT |
| 99 | TGCACTGCTGCAGAT |
| 100 | TGCACTGCACGAT |
| 101 | TGCACTGCACATGAT |
| 102 | TGCACTGCAGATGAT |
| 103 | TGCACTGCAGCTGAT |
| 104 | TGCACTGCAGCAGAT |
| 105 | TGCACTGCATATGAT |
| 106 | TGCACTGCATCTGAT |
| 107 | TGCACTGCATCAGAT |
| 108 | TGCACTGCATGTGAT |
| 109 | TGCACTGCATGAGAT |
| 110 | TGCACTGCATGCGAT |
| 111 | TGCACACGATGAT |
| 112 | TGCACACGCTGAT |
| 113 | TGCACACGCAGAT |
| 114 | TGCACACTATGAT |
| 115 | TGCACACTCTGAT |
| 116 | TGCACACTCAGAT |
| 117 | TGCACACTGTGAT |
| 118 | TGCACACTGAGAT |
| 119 | TGCACACTGCGAT |
| 120 | TGCACACTGCATGAT |
| 121 | TGCACACACTGAT |
| 122 | TGCACACACAGAT |
| 123 | TGCACACAGTGAT |
| 124 | TGCACACAGAGAT |
| 125 | TGCACACAGCGAT |
| 126 | TGCACACAGCATGAT |
| 127 | TGCACACATAGAT |
| 128 | TGCACACATCGAT |
| 129 | TGCACACATCATGAT |
| 130 | TGCACACATGATGAT |
| 131 | TGCACACATGCTGAT |
| 132 | TGCACACATGCAGAT |
| 133 | TGCACAGTATGAT |
| 134 | TGCACAGTCTGAT |
| 135 | TGCACAGTCAGAT |
| 136 | TGCACAGTGTGAT |
| 137 | TGCACAGTGAGAT |
| 138 | TGCACAGTGCGAT |
| 139 | TGCACAGTGCATGAT |
| 140 | TGCACAGACTGAT |
| 141 | TGCACAGACAGAT |
| 142 | TGCACAGAGTGAT |
| 143 | TGCACAGAGAGAT |
| 144 | TGCACAGAGCGAT |
| 145 | TGCACAGAGCATGAT |
| 146 | TGCACAGATAGAT |
| 147 | TGCACAGATCGAT |
| 148 | TGCACAGATCATGAT |
| 149 | TGCACAGATGATGAT |
| 150 | TGCACAGATGCTGAT |
| 151 | TGCACAGATGCAGAT |
| 152 | TGCACAGCGTGAT |
| 153 | TGCACAGCGAGAT |
| 154 | TGCACAGCGCGAT |
| 155 | TGCACAGCGCATGAT |
| 156 | TGCACAGCTAGAT |
| 157 | TGCACAGCTCGAT |
| 158 | TGCACAGCTCATGAT |
| 159 | TGCACAGCTGATGAT |
| 160 | TGCACAGCTGCTGAT |
| 161 | TGCACAGCTGCAGAT |
| 162 | TGCACAGCACGAT |
| 163 | TGCACAGCACATGAT |
| 164 | TGCACAGCAGATGAT |
| 165 | TGCACAGCAGCTGAT |
| 166 | TGCACAGCAGCAGAT |
| 167 | TGCACAGCATATGAT |
| 168 | TGCACAGCATCTGAT |
| 169 | TGCACAGCATCAGAT |
| 170 | TGCACAGCATGTGAT |
| 171 | TGCACAGCATGAGAT |
| 172 | TGCACAGCATGCGAT |
| 173 | TGCACATACTGAT |
| 174 | TGCACATACAGAT |
| 175 | TGCACATAGTGAT |
| 176 | TGCACATAGAGAT |
| 177 | TGCACATAGCGAT |
| 178 | TGCACATAGCATGAT |
| 179 | TGCACATATAGAT |
| 180 | TGCACATATCGAT |
| 181 | TGCACATATCATGAT |
| 182 | TGCACATATGATGAT |
| 183 | TGCACATATGCTGAT |
| 184 | TGCACATATGCAGAT |
| 185 | TGCACATCGTGAT |
| 186 | TGCACATCGAGAT |
| 187 | TGCACATCGCGAT |
| 188 | TGCACATCGCATGAT |
| 189 | TGCACATCTAGAT |
| 190 | TGCACATCTCGAT |
| 191 | TGCACATCTCATGAT |
| 192 | TGCACATCTGATGAT |
| 193 | TGCACATCTGCTGAT |
| 194 | TGCACATCTGCAGAT |
| 195 | TGCACATCACGAT |
| 196 | TGCACATCACATGAT |
| 197 | TGCACATCAGATGAT |
| 198 | TGCACATCAGCTGAT |
| 199 | TGCACATCAGCAGAT |
| 200 | TGCACATCATATGAT |
| 201 | TGCACATCATCTGAT |
| 202 | TGCACATCATCAGAT |
| 203 | TGCACATCATGTGAT |
| 204 | TGCACATCATGAGAT |
| 205 | TGCACATCATGCGAT |
| 206 | TGCACATGTAGAT |
| 207 | TGCACATGTCGAT |
| 208 | TGCACATGTCATGAT |
| 209 | TGCACATGTGATGAT |
| 210 | TGCACATGTGCTGAT |
| 211 | TGCACATGTGCAGAT |
| 212 | TGCACATGACGAT |
| 213 | TGCACATGACATGAT |
| 214 | TGCACATGAGATGAT |
| 215 | TGCACATGAGCTGAT |
| 216 | TGCACATGAGCAGAT |
| 217 | TGCACATGATATGAT |
| 218 | TGCACATGATCTGAT |
| 219 | TGCACATGATCAGAT |
| 220 | TGCACATGATGTGAT |
| 221 | TGCACATGATGAGAT |
| 222 | TGCACATGATGCGAT |
| 223 | TGCACATGCGATGAT |
| 224 | TGCACATGCGCTGAT |
| 225 | TGCACATGCGCAGAT |
| 226 | TGCACATGCTATGAT |
| 227 | TGCACATGCTCTGAT |
| 228 | TGCACATGCTCAGAT |
| 229 | TGCACATGCTGTGAT |
| 230 | TGCACATGCTGAGAT |
| 231 | TGCACATGCTGCGAT |
| 232 | TGCACATGCACTGAT |
| 233 | TGCACATGCACAGAT |
| 234 | TGCACATGCAGTGAT |
| 235 | TGCACATGCAGAGAT |
| 236 | TGCACATGCAGCGAT |
| 237 | TGCACATGCATAGAT |
| 238 | TGCACATGCATCGAT |

Table 3B provides flowgrams (e.g., vectors of flow cycle values) for each barcode sequence (SEQ ID NOs: 1-238) determined in accordance with these requirements.

TABLE 3B
List of example barcode sequences (represented by their corresponding SEQ ID
NOs) and the flow cycle values resultant from 20 flow cycles, where the edit
distance between each possible pair of barcode sequences is at least 2.

| SEQ | | | | | | | | | | | | | | | | | | | | | |
|---|---|---|---|---|---|---|---|---|---|---|---|---|---|---|---|---|---|---|---|---|---|
| ID | 0 | 1 | 2 | 3 | 4 | 5 | 6 | 7 | 8 | 9 | 10 | 11 | 12 | 13 | 14 | 15 | 16 | 17 | 18 | 19 | 20 |
| NO: | T | G | C | A | T | G | C | A | T | G | C | A | T | G | C | A | T | G | C | A | T |
| 1 | 1 | 1 | 1 | 1 | 0 | 0 | 1 | 0 | 0 | 1 | 0 | 0 | 1 | 0 | 1 | 1 | 1 | 1 | 0 | 1 | 1 |
| 2 | 1 | 1 | 1 | 1 | 0 | 0 | 1 | 0 | 0 | 1 | 0 | 0 | 1 | 1 | 0 | 1 | 1 | 1 | 0 | 1 | 1 |
| 3 | 1 | 1 | 1 | 1 | 0 | 0 | 1 | 0 | 0 | 1 | 0 | 0 | 1 | 1 | 1 | 0 | 1 | 1 | 0 | 1 | 1 |
| 4 | 1 | 1 | 1 | 1 | 0 | 0 | 1 | 0 | 0 | 1 | 0 | 0 | 1 | 1 | 1 | 1 | 0 | 1 | 0 | 1 | 1 |
| 5 | 1 | 1 | 1 | 1 | 0 | 0 | 1 | 0 | 0 | 1 | 0 | 1 | 0 | 0 | 1 | 1 | 1 | 1 | 0 | 1 | 1 |
| 6 | 1 | 1 | 1 | 1 | 0 | 0 | 1 | 0 | 0 | 1 | 0 | 1 | 0 | 1 | 0 | 1 | 1 | 1 | 0 | 1 | 1 |
| 7 | 1 | 1 | 1 | 1 | 0 | 0 | 1 | 0 | 0 | 1 | 0 | 1 | 0 | 1 | 1 | 0 | 1 | 1 | 0 | 1 | 1 |
| 8 | 1 | 1 | 1 | 1 | 0 | 0 | 1 | 0 | 0 | 1 | 0 | 1 | 0 | 1 | 1 | 1 | 0 | 1 | 0 | 1 | 1 |
| 9 | 1 | 1 | 1 | 1 | 0 | 0 | 1 | 0 | 0 | 1 | 0 | 1 | 1 | 0 | 0 | 1 | 1 | 1 | 0 | 1 | 1 |
| 10 | 1 | 1 | 1 | 1 | 0 | 0 | 1 | 0 | 0 | 1 | 0 | 1 | 1 | 0 | 1 | 0 | 1 | 1 | 0 | 1 | 1 |
| 11 | 1 | 1 | 1 | 1 | 0 | 0 | 1 | 0 | 0 | 1 | 0 | 1 | 1 | 0 | 1 | 1 | 0 | 1 | 0 | 1 | 1 |
| 12 | 1 | 1 | 1 | 1 | 0 | 0 | 1 | 0 | 0 | 1 | 0 | 1 | 1 | 1 | 0 | 0 | 1 | 1 | 0 | 1 | 1 |
| 13 | 1 | 1 | 1 | 1 | 0 | 0 | 1 | 0 | 0 | 1 | 0 | 1 | 1 | 1 | 0 | 1 | 0 | 1 | 0 | 1 | 1 |
| 14 | 1 | 1 | 1 | 1 | 0 | 0 | 1 | 0 | 0 | 1 | 0 | 1 | 1 | 1 | 1 | 0 | 0 | 1 | 0 | 1 | 1 |
| 15 | 1 | 1 | 1 | 1 | 0 | 0 | 1 | 0 | 0 | 1 | 0 | 1 | 1 | 1 | 1 | 1 | 1 | 1 | 0 | 1 | 1 |
| 16 | 1 | 1 | 1 | 1 | 0 | 0 | 1 | 0 | 0 | 1 | 1 | 0 | 0 | 1 | 0 | 1 | 1 | 1 | 0 | 1 | 1 |
| 17 | 1 | 1 | 1 | 1 | 0 | 0 | 1 | 0 | 0 | 1 | 1 | 0 | 0 | 1 | 1 | 0 | 1 | 1 | 0 | 1 | 1 |
| 18 | 1 | 1 | 1 | 1 | 0 | 0 | 1 | 0 | 0 | 1 | 1 | 0 | 0 | 1 | 1 | 1 | 0 | 1 | 0 | 1 | 1 |
| 19 | 1 | 1 | 1 | 1 | 0 | 0 | 1 | 0 | 0 | 1 | 1 | 0 | 1 | 0 | 0 | 1 | 1 | 1 | 0 | 1 | 1 |
| 20 | 1 | 1 | 1 | 1 | 0 | 0 | 1 | 0 | 0 | 1 | 1 | 0 | 1 | 0 | 1 | 0 | 1 | 1 | 0 | 1 | 1 |
| 21 | 1 | 1 | 1 | 1 | 0 | 0 | 1 | 0 | 0 | 1 | 1 | 0 | 1 | 0 | 1 | 1 | 0 | 1 | 0 | 1 | 1 |
| 22 | 1 | 1 | 1 | 1 | 0 | 0 | 1 | 0 | 0 | 1 | 1 | 0 | 1 | 1 | 0 | 0 | 1 | 1 | 0 | 1 | 1 |
| 23 | 1 | 1 | 1 | 1 | 0 | 0 | 1 | 0 | 0 | 1 | 1 | 0 | 1 | 1 | 0 | 1 | 0 | 1 | 0 | 1 | 1 |
| 24 | 1 | 1 | 1 | 1 | 0 | 0 | 1 | 0 | 0 | 1 | 1 | 0 | 1 | 1 | 1 | 0 | 0 | 1 | 0 | 1 | 1 |
| 25 | 1 | 1 | 1 | 1 | 0 | 0 | 1 | 0 | 0 | 1 | 1 | 0 | 1 | 1 | 1 | 1 | 1 | 1 | 0 | 1 | 1 |
| 26 | 1 | 1 | 1 | 1 | 0 | 0 | 1 | 0 | 0 | 1 | 1 | 1 | 0 | 0 | 1 | 0 | 1 | 1 | 0 | 1 | 1 |
| 27 | 1 | 1 | 1 | 1 | 0 | 0 | 1 | 0 | 0 | 1 | 1 | 1 | 0 | 0 | 1 | 1 | 0 | 1 | 0 | 1 | 1 |
| 28 | 1 | 1 | 1 | 1 | 0 | 0 | 1 | 0 | 0 | 1 | 1 | 1 | 0 | 1 | 0 | 0 | 1 | 1 | 0 | 1 | 1 |
| 29 | 1 | 1 | 1 | 1 | 0 | 0 | 1 | 0 | 0 | 1 | 1 | 1 | 0 | 1 | 0 | 1 | 0 | 1 | 0 | 1 | 1 |
| 30 | 1 | 1 | 1 | 1 | 0 | 0 | 1 | 0 | 0 | 1 | 1 | 1 | 0 | 1 | 1 | 0 | 0 | 1 | 0 | 1 | 1 |
| 31 | 1 | 1 | 1 | 1 | 0 | 0 | 1 | 0 | 0 | 1 | 1 | 1 | 0 | 1 | 1 | 1 | 1 | 1 | 0 | 1 | 1 |
| 32 | 1 | 1 | 1 | 1 | 0 | 0 | 1 | 0 | 0 | 1 | 1 | 1 | 1 | 0 | 0 | 1 | 0 | 1 | 0 | 1 | 1 |
| 33 | 1 | 1 | 1 | 1 | 0 | 0 | 1 | 0 | 0 | 1 | 1 | 1 | 1 | 0 | 1 | 0 | 0 | 1 | 0 | 1 | 1 |
| 34 | 1 | 1 | 1 | 1 | 0 | 0 | 1 | 0 | 0 | 1 | 1 | 1 | 1 | 0 | 1 | 1 | 1 | 1 | 0 | 1 | 1 |
| 35 | 1 | 1 | 1 | 1 | 0 | 0 | 1 | 0 | 0 | 1 | 1 | 1 | 1 | 1 | 0 | 1 | 1 | 1 | 0 | 1 | 1 |
| 36 | 1 | 1 | 1 | 1 | 0 | 0 | 1 | 0 | 0 | 1 | 1 | 1 | 1 | 1 | 1 | 0 | 1 | 1 | 0 | 1 | 1 |
| 37 | 1 | 1 | 1 | 1 | 0 | 0 | 1 | 0 | 0 | 1 | 1 | 1 | 1 | 1 | 1 | 1 | 0 | 1 | 0 | 1 | 1 |
| 38 | 1 | 1 | 1 | 1 | 0 | 0 | 1 | 0 | 1 | 0 | 0 | 1 | 0 | 0 | 1 | 1 | 1 | 1 | 0 | 1 | 1 |
| 39 | 1 | 1 | 1 | 1 | 0 | 0 | 1 | 0 | 1 | 0 | 0 | 1 | 0 | 1 | 0 | 1 | 1 | 1 | 0 | 1 | 1 |
| 40 | 1 | 1 | 1 | 1 | 0 | 0 | 1 | 0 | 1 | 0 | 0 | 1 | 0 | 1 | 1 | 0 | 1 | 1 | 0 | 1 | 1 |
| 41 | 1 | 1 | 1 | 1 | 0 | 0 | 1 | 0 | 1 | 0 | 0 | 1 | 0 | 1 | 1 | 1 | 0 | 1 | 0 | 1 | 1 |
| 42 | 1 | 1 | 1 | 1 | 0 | 0 | 1 | 0 | 1 | 0 | 0 | 1 | 1 | 0 | 0 | 1 | 1 | 1 | 0 | 1 | 1 |
| 43 | 1 | 1 | 1 | 1 | 0 | 0 | 1 | 0 | 1 | 0 | 0 | 1 | 1 | 0 | 1 | 0 | 1 | 1 | 0 | 1 | 1 |
| 44 | 1 | 1 | 1 | 1 | 0 | 0 | 1 | 0 | 1 | 0 | 0 | 1 | 1 | 0 | 1 | 1 | 0 | 1 | 0 | 1 | 1 |
| 45 | 1 | 1 | 1 | 1 | 0 | 0 | 1 | 0 | 1 | 0 | 0 | 1 | 1 | 1 | 0 | 0 | 1 | 1 | 0 | 1 | 1 |
| 46 | 1 | 1 | 1 | 1 | 0 | 0 | 1 | 0 | 1 | 0 | 0 | 1 | 1 | 1 | 0 | 1 | 0 | 1 | 0 | 1 | 1 |
| 47 | 1 | 1 | 1 | 1 | 0 | 0 | 1 | 0 | 1 | 0 | 0 | 1 | 1 | 1 | 1 | 0 | 0 | 1 | 0 | 1 | 1 |
| 48 | 1 | 1 | 1 | 1 | 0 | 0 | 1 | 0 | 1 | 0 | 0 | 1 | 1 | 1 | 1 | 1 | 1 | 1 | 0 | 1 | 1 |
| 49 | 1 | 1 | 1 | 1 | 0 | 0 | 1 | 0 | 1 | 0 | 1 | 0 | 0 | 1 | 0 | 1 | 1 | 1 | 0 | 1 | 1 |
| 50 | 1 | 1 | 1 | 1 | 0 | 0 | 1 | 0 | 1 | 0 | 1 | 0 | 0 | 1 | 1 | 0 | 1 | 1 | 0 | 1 | 1 |
| 51 | 1 | 1 | 1 | 1 | 0 | 0 | 1 | 0 | 1 | 0 | 1 | 0 | 0 | 1 | 1 | 1 | 0 | 1 | 0 | 1 | 1 |
| 52 | 1 | 1 | 1 | 1 | 0 | 0 | 1 | 0 | 1 | 0 | 1 | 0 | 1 | 0 | 0 | 1 | 1 | 1 | 0 | 1 | 1 |
| 53 | 1 | 1 | 1 | 1 | 0 | 0 | 1 | 0 | 1 | 0 | 1 | 0 | 1 | 0 | 1 | 0 | 1 | 1 | 0 | 1 | 1 |
| 54 | 1 | 1 | 1 | 1 | 0 | 0 | 1 | 0 | 1 | 0 | 1 | 0 | 1 | 0 | 1 | 1 | 0 | 1 | 0 | 1 | 1 |
| 55 | 1 | 1 | 1 | 1 | 0 | 0 | 1 | 0 | 1 | 0 | 1 | 0 | 1 | 1 | 0 | 0 | 1 | 1 | 0 | 1 | 1 |
| 56 | 1 | 1 | 1 | 1 | 0 | 0 | 1 | 0 | 1 | 0 | 1 | 0 | 1 | 1 | 0 | 1 | 0 | 1 | 0 | 1 | 1 |
| 57 | 1 | 1 | 1 | 1 | 0 | 0 | 1 | 0 | 1 | 0 | 1 | 0 | 1 | 1 | 1 | 0 | 0 | 1 | 0 | 1 | 1 |
| 58 | 1 | 1 | 1 | 1 | 0 | 0 | 1 | 0 | 1 | 0 | 1 | 0 | 1 | 1 | 1 | 1 | 1 | 1 | 0 | 1 | 1 |
| 59 | 1 | 1 | 1 | 1 | 0 | 0 | 1 | 0 | 1 | 0 | 1 | 1 | 0 | 0 | 1 | 0 | 1 | 1 | 0 | 1 | 1 |
| 60 | 1 | 1 | 1 | 1 | 0 | 0 | 1 | 0 | 1 | 0 | 1 | 1 | 0 | 0 | 1 | 1 | 0 | 1 | 0 | 1 | 1 |
| 61 | 1 | 1 | 1 | 1 | 0 | 0 | 1 | 0 | 1 | 0 | 1 | 1 | 0 | 1 | 0 | 0 | 1 | 1 | 0 | 1 | 1 |
| 62 | 1 | 1 | 1 | 1 | 0 | 0 | 1 | 0 | 1 | 0 | 1 | 1 | 0 | 1 | 0 | 1 | 0 | 1 | 0 | 1 | 1 |
| 63 | 1 | 1 | 1 | 1 | 0 | 0 | 1 | 0 | 1 | 0 | 1 | 1 | 0 | 1 | 1 | 0 | 0 | 1 | 0 | 1 | 1 |
| 64 | 1 | 1 | 1 | 1 | 0 | 0 | 1 | 0 | 1 | 0 | 1 | 1 | 0 | 1 | 1 | 1 | 1 | 1 | 0 | 1 | 1 |
| 65 | 1 | 1 | 1 | 1 | 0 | 0 | 1 | 0 | 1 | 0 | 1 | 1 | 1 | 0 | 0 | 1 | 0 | 1 | 0 | 1 | 1 |
| 66 | 1 | 1 | 1 | 1 | 0 | 0 | 1 | 0 | 1 | 0 | 1 | 1 | 1 | 0 | 1 | 0 | 0 | 1 | 0 | 1 | 1 |
| 67 | 1 | 1 | 1 | 1 | 0 | 0 | 1 | 0 | 1 | 0 | 1 | 1 | 1 | 0 | 1 | 1 | 1 | 1 | 0 | 1 | 1 |
| 68 | 1 | 1 | 1 | 1 | 0 | 0 | 1 | 0 | 1 | 0 | 1 | 1 | 1 | 1 | 0 | 1 | 1 | 1 | 0 | 1 | 1 |
| 69 | 1 | 1 | 1 | 1 | 0 | 0 | 1 | 0 | 1 | 0 | 1 | 1 | 1 | 1 | 1 | 0 | 1 | 1 | 0 | 1 | 1 |
| 70 | 1 | 1 | 1 | 1 | 0 | 0 | 1 | 0 | 1 | 0 | 1 | 1 | 1 | 1 | 1 | 1 | 0 | 1 | 0 | 1 | 1 |
| 71 | 1 | 1 | 1 | 1 | 0 | 0 | 1 | 0 | 1 | 1 | 0 | 0 | 1 | 0 | 0 | 1 | 1 | 1 | 0 | 1 | 1 |
| 72 | 1 | 1 | 1 | 1 | 0 | 0 | 1 | 0 | 1 | 1 | 0 | 0 | 1 | 0 | 1 | 0 | 1 | 1 | 0 | 1 | 1 |
| 73 | 1 | 1 | 1 | 1 | 0 | 0 | 1 | 0 | 1 | 1 | 0 | 0 | 1 | 0 | 1 | 1 | 0 | 1 | 0 | 1 | 1 |
| 74 | 1 | 1 | 1 | 1 | 0 | 0 | 1 | 0 | 1 | 1 | 0 | 0 | 1 | 1 | 0 | 0 | 1 | 1 | 0 | 1 | 1 |
| 75 | 1 | 1 | 1 | 1 | 0 | 0 | 1 | 0 | 1 | 1 | 0 | 0 | 1 | 1 | 0 | 1 | 0 | 1 | 0 | 1 | 1 |
| 76 | 1 | 1 | 1 | 1 | 0 | 0 | 1 | 0 | 1 | 1 | 0 | 0 | 1 | 1 | 1 | 0 | 0 | 1 | 0 | 1 | 1 |
| 77 | 1 | 1 | 1 | 1 | 0 | 0 | 1 | 0 | 1 | 1 | 0 | 0 | 1 | 1 | 1 | 1 | 1 | 1 | 0 | 1 | 1 |
| 78 | 1 | 1 | 1 | 1 | 0 | 0 | 1 | 0 | 1 | 1 | 0 | 1 | 0 | 0 | 1 | 0 | 1 | 1 | 0 | 1 | 1 |
| 79 | 1 | 1 | 1 | 1 | 0 | 0 | 1 | 0 | 1 | 1 | 0 | 1 | 0 | 0 | 1 | 1 | 0 | 1 | 0 | 1 | 1 |
| 80 | 1 | 1 | 1 | 1 | 0 | 0 | 1 | 0 | 1 | 1 | 0 | 1 | 0 | 1 | 0 | 0 | 1 | 1 | 0 | 1 | 1 |
| 81 | 1 | 1 | 1 | 1 | 0 | 0 | 1 | 0 | 1 | 1 | 0 | 1 | 0 | 1 | 0 | 1 | 0 | 1 | 0 | 1 | 1 |
| 82 | 1 | 1 | 1 | 1 | 0 | 0 | 1 | 0 | 1 | 1 | 0 | 1 | 0 | 1 | 1 | 0 | 0 | 1 | 0 | 1 | 1 |
| 83 | 1 | 1 | 1 | 1 | 0 | 0 | 1 | 0 | 1 | 1 | 0 | 1 | 0 | 1 | 1 | 1 | 1 | 1 | 0 | 1 | 1 |
| 84 | 1 | 1 | 1 | 1 | 0 | 0 | 1 | 0 | 1 | 1 | 0 | 1 | 1 | 0 | 0 | 1 | 0 | 1 | 0 | 1 | 1 |
| 85 | 1 | 1 | 1 | 1 | 0 | 0 | 1 | 0 | 1 | 1 | 0 | 1 | 1 | 0 | 1 | 0 | 0 | 1 | 0 | 1 | 1 |
| 86 | 1 | 1 | 1 | 1 | 0 | 0 | 1 | 0 | 1 | 1 | 0 | 1 | 1 | 0 | 1 | 1 | 1 | 1 | 0 | 1 | 1 |
| 87 | 1 | 1 | 1 | 1 | 0 | 0 | 1 | 0 | 1 | 1 | 0 | 1 | 1 | 1 | 0 | 1 | 1 | 1 | 0 | 1 | 1 |
| 88 | 1 | 1 | 1 | 1 | 0 | 0 | 1 | 0 | 1 | 1 | 0 | 1 | 1 | 1 | 1 | 0 | 1 | 1 | 0 | 1 | 1 |
| 89 | 1 | 1 | 1 | 1 | 0 | 0 | 1 | 0 | 1 | 1 | 0 | 1 | 1 | 1 | 1 | 1 | 0 | 1 | 0 | 1 | 1 |
| 90 | 1 | 1 | 1 | 1 | 0 | 0 | 1 | 0 | 1 | 1 | 1 | 0 | 0 | 1 | 0 | 0 | 1 | 1 | 0 | 1 | 1 |
| 91 | 1 | 1 | 1 | 1 | 0 | 0 | 1 | 0 | 1 | 1 | 1 | 0 | 0 | 1 | 0 | 1 | 0 | 1 | 0 | 1 | 1 |
| 92 | 1 | 1 | 1 | 1 | 0 | 0 | 1 | 0 | 1 | 1 | 1 | 0 | 0 | 1 | 1 | 0 | 0 | 1 | 0 | 1 | 1 |
| 93 | 1 | 1 | 1 | 1 | 0 | 0 | 1 | 0 | 1 | 1 | 1 | 0 | 0 | 1 | 1 | 1 | 1 | 1 | 0 | 1 | 1 |
| 94 | 1 | 1 | 1 | 1 | 0 | 0 | 1 | 0 | 1 | 1 | 1 | 0 | 1 | 0 | 0 | 1 | 0 | 1 | 0 | 1 | 1 |
| 95 | 1 | 1 | 1 | 1 | 0 | 0 | 1 | 0 | 1 | 1 | 1 | 0 | 1 | 0 | 1 | 0 | 0 | 1 | 0 | 1 | 1 |
| 96 | 1 | 1 | 1 | 1 | 0 | 0 | 1 | 0 | 1 | 1 | 1 | 0 | 1 | 0 | 1 | 1 | 1 | 1 | 0 | 1 | 1 |
| 97 | 1 | 1 | 1 | 1 | 0 | 0 | 1 | 0 | 1 | 1 | 1 | 0 | 1 | 1 | 0 | 1 | 1 | 1 | 0 | 1 | 1 |
| 98 | 1 | 1 | 1 | 1 | 0 | 0 | 1 | 0 | 1 | 1 | 1 | 0 | 1 | 1 | 1 | 0 | 1 | 1 | 0 | 1 | 1 |
| 99 | 1 | 1 | 1 | 1 | 0 | 0 | 1 | 0 | 1 | 1 | 1 | 0 | 1 | 1 | 1 | 1 | 0 | 1 | 0 | 1 | 1 |
| 100 | 1 | 1 | 1 | 1 | 0 | 0 | 1 | 0 | 1 | 1 | 1 | 1 | 0 | 0 | 1 | 0 | 0 | 1 | 0 | 1 | 1 |
| 101 | 1 | 1 | 1 | 1 | 0 | 0 | 1 | 0 | 1 | 1 | 1 | 1 | 0 | 0 | 1 | 1 | 1 | 1 | 0 | 1 | 1 |
| 102 | 1 | 1 | 1 | 1 | 0 | 0 | 1 | 0 | 1 | 1 | 1 | 1 | 0 | 1 | 0 | 1 | 1 | 1 | 0 | 1 | 1 |
| 103 | 1 | 1 | 1 | 1 | 0 | 0 | 1 | 0 | 1 | 1 | 1 | 1 | 0 | 1 | 1 | 0 | 1 | 1 | 0 | 1 | 1 |
| 104 | 1 | 1 | 1 | 1 | 0 | 0 | 1 | 0 | 1 | 1 | 1 | 1 | 0 | 1 | 1 | 1 | 0 | 1 | 0 | 1 | 1 |
| 105 | 1 | 1 | 1 | 1 | 0 | 0 | 1 | 0 | 1 | 1 | 1 | 1 | 1 | 0 | 0 | 1 | 1 | 1 | 0 | 1 | 1 |
| 106 | 1 | 1 | 1 | 1 | 0 | 0 | 1 | 0 | 1 | 1 | 1 | 1 | 1 | 0 | 1 | 0 | 1 | 1 | 0 | 1 | 1 |
| 107 | 1 | 1 | 1 | 1 | 0 | 0 | 1 | 0 | 1 | 1 | 1 | 1 | 1 | 0 | 1 | 1 | 0 | 1 | 0 | 1 | 1 |
| 108 | 1 | 1 | 1 | 1 | 0 | 0 | 1 | 0 | 1 | 1 | 1 | 1 | 1 | 1 | 0 | 0 | 1 | 1 | 0 | 1 | 1 |
| 109 | 1 | 1 | 1 | 1 | 0 | 0 | 1 | 0 | 1 | 1 | 1 | 1 | 1 | 1 | 0 | 1 | 0 | 1 | 0 | 1 | 1 |
| 110 | 1 | 1 | 1 | 1 | 0 | 0 | 1 | 0 | 1 | 1 | 1 | 1 | 1 | 1 | 1 | 0 | 0 | 1 | 0 | 1 | 1 |
| 111 | 1 | 1 | 1 | 1 | 0 | 0 | 1 | 1 | 0 | 0 | 1 | 0 | 0 | 1 | 0 | 1 | 1 | 1 | 0 | 1 | 1 |
| 112 | 1 | 1 | 1 | 1 | 0 | 0 | 1 | 1 | 0 | 0 | 1 | 0 | 0 | 1 | 1 | 0 | 1 | 1 | 0 | 1 | 1 |
| 113 | 1 | 1 | 1 | 1 | 0 | 0 | 1 | 1 | 0 | 0 | 1 | 0 | 0 | 1 | 1 | 1 | 0 | 1 | 0 | 1 | 1 |
| 114 | 1 | 1 | 1 | 1 | 0 | 0 | 1 | 1 | 0 | 0 | 1 | 0 | 1 | 0 | 0 | 1 | 1 | 1 | 0 | 1 | 1 |
| 115 | 1 | 1 | 1 | 1 | 0 | 0 | 1 | 1 | 0 | 0 | 1 | 0 | 1 | 0 | 1 | 0 | 1 | 1 | 0 | 1 | 1 |
| 116 | 1 | 1 | 1 | 1 | 0 | 0 | 1 | 1 | 0 | 0 | 1 | 0 | 1 | 0 | 1 | 1 | 0 | 1 | 0 | 1 | 1 |
| 117 | 1 | 1 | 1 | 1 | 0 | 0 | 1 | 1 | 0 | 0 | 1 | 0 | 1 | 1 | 0 | 0 | 1 | 1 | 0 | 1 | 1 |
| 118 | 1 | 1 | 1 | 1 | 0 | 0 | 1 | 1 | 0 | 0 | 1 | 0 | 1 | 1 | 0 | 1 | 0 | 1 | 0 | 1 | 1 |
| 119 | 1 | 1 | 1 | 1 | 0 | 0 | 1 | 1 | 0 | 0 | 1 | 0 | 1 | 1 | 1 | 0 | 0 | 1 | 0 | 1 | 1 |
| 120 | 1 | 1 | 1 | 1 | 0 | 0 | 1 | 1 | 0 | 0 | 1 | 0 | 1 | 1 | 1 | 1 | 1 | 1 | 0 | 1 | 1 |
| 121 | 1 | 1 | 1 | 1 | 0 | 0 | 1 | 1 | 0 | 0 | 1 | 1 | 0 | 0 | 1 | 0 | 1 | 1 | 0 | 1 | 1 |
| 122 | 1 | 1 | 1 | 1 | 0 | 0 | 1 | 1 | 0 | 0 | 1 | 1 | 0 | 0 | 1 | 1 | 0 | 1 | 0 | 1 | 1 |
| 123 | 1 | 1 | 1 | 1 | 0 | 0 | 1 | 1 | 0 | 0 | 1 | 1 | 0 | 1 | 0 | 0 | 1 | 1 | 0 | 1 | 1 |
| 124 | 1 | 1 | 1 | 1 | 0 | 0 | 1 | 1 | 0 | 0 | 1 | 1 | 0 | 1 | 0 | 1 | 0 | 1 | 0 | 1 | 1 |
| 125 | 1 | 1 | 1 | 1 | 0 | 0 | 1 | 1 | 0 | 0 | 1 | 1 | 0 | 1 | 1 | 0 | 0 | 1 | 0 | 1 | 1 |
| 126 | 1 | 1 | 1 | 1 | 0 | 0 | 1 | 1 | 0 | 0 | 1 | 1 | 0 | 1 | 1 | 1 | 1 | 1 | 0 | 1 | 1 |
| 127 | 1 | 1 | 1 | 1 | 0 | 0 | 1 | 1 | 0 | 0 | 1 | 1 | 1 | 0 | 0 | 1 | 0 | 1 | 0 | 1 | 1 |
| 128 | 1 | 1 | 1 | 1 | 0 | 0 | 1 | 1 | 0 | 0 | 1 | 1 | 1 | 0 | 1 | 0 | 0 | 1 | 0 | 1 | 1 |
| 129 | 1 | 1 | 1 | 1 | 0 | 0 | 1 | 1 | 0 | 0 | 1 | 1 | 1 | 0 | 1 | 1 | 1 | 1 | 0 | 1 | 1 |
| 130 | 1 | 1 | 1 | 1 | 0 | 0 | 1 | 1 | 0 | 0 | 1 | 1 | 1 | 1 | 0 | 1 | 1 | 1 | 0 | 1 | 1 |
| 131 | 1 | 1 | 1 | 1 | 0 | 0 | 1 | 1 | 0 | 0 | 1 | 1 | 1 | 1 | 1 | 0 | 1 | 1 | 0 | 1 | 1 |
| 132 | 1 | 1 | 1 | 1 | 0 | 0 | 1 | 1 | 0 | 0 | 1 | 1 | 1 | 1 | 1 | 1 | 0 | 1 | 0 | 1 | 1 |
| 133 | 1 | 1 | 1 | 1 | 0 | 0 | 1 | 1 | 0 | 1 | 0 | 0 | 1 | 0 | 0 | 1 | 1 | 1 | 0 | 1 | 1 |
| 134 | 1 | 1 | 1 | 1 | 0 | 0 | 1 | 1 | 0 | 1 | 0 | 0 | 1 | 0 | 1 | 0 | 1 | 1 | 0 | 1 | 1 |
| 135 | 1 | 1 | 1 | 1 | 0 | 0 | 1 | 1 | 0 | 1 | 0 | 0 | 1 | 0 | 1 | 1 | 0 | 1 | 0 | 1 | 1 |
| 136 | 1 | 1 | 1 | 1 | 0 | 0 | 1 | 1 | 0 | 1 | 0 | 0 | 1 | 1 | 0 | 0 | 1 | 1 | 0 | 1 | 1 |
| 137 | 1 | 1 | 1 | 1 | 0 | 0 | 1 | 1 | 0 | 1 | 0 | 0 | 1 | 1 | 0 | 1 | 0 | 1 | 0 | 1 | 1 |
| 138 | 1 | 1 | 1 | 1 | 0 | 0 | 1 | 1 | 0 | 1 | 0 | 0 | 1 | 1 | 1 | 0 | 0 | 1 | 0 | 1 | 1 |
| 139 | 1 | 1 | 1 | 1 | 0 | 0 | 1 | 1 | 0 | 1 | 0 | 0 | 1 | 1 | 1 | 1 | 1 | 1 | 0 | 1 | 1 |
| 140 | 1 | 1 | 1 | 1 | 0 | 0 | 1 | 1 | 0 | 1 | 0 | 1 | 0 | 0 | 1 | 0 | 1 | 1 | 0 | 1 | 1 |
| 141 | 1 | 1 | 1 | 1 | 0 | 0 | 1 | 1 | 0 | 1 | 0 | 1 | 0 | 0 | 1 | 1 | 0 | 1 | 0 | 1 | 1 |
| 142 | 1 | 1 | 1 | 1 | 0 | 0 | 1 | 1 | 0 | 1 | 0 | 1 | 0 | 1 | 0 | 0 | 1 | 1 | 0 | 1 | 1 |
| 143 | 1 | 1 | 1 | 1 | 0 | 0 | 1 | 1 | 0 | 1 | 0 | 1 | 0 | 1 | 0 | 1 | 0 | 1 | 0 | 1 | 1 |
| 144 | 1 | 1 | 1 | 1 | 0 | 0 | 1 | 1 | 0 | 1 | 0 | 1 | 0 | 1 | 1 | 0 | 0 | 1 | 0 | 1 | 1 |
| 145 | 1 | 1 | 1 | 1 | 0 | 0 | 1 | 1 | 0 | 1 | 0 | 1 | 0 | 1 | 1 | 1 | 1 | 1 | 0 | 1 | 1 |
| 146 | 1 | 1 | 1 | 1 | 0 | 0 | 1 | 1 | 0 | 1 | 0 | 1 | 1 | 0 | 0 | 1 | 0 | 1 | 0 | 1 | 1 |
| 147 | 1 | 1 | 1 | 1 | 0 | 0 | 1 | 1 | 0 | 1 | 0 | 1 | 1 | 0 | 1 | 0 | 0 | 1 | 0 | 1 | 1 |
| 148 | 1 | 1 | 1 | 1 | 0 | 0 | 1 | 1 | 0 | 1 | 0 | 1 | 1 | 0 | 1 | 1 | 1 | 1 | 0 | 1 | 1 |
| 149 | 1 | 1 | 1 | 1 | 0 | 0 | 1 | 1 | 0 | 1 | 0 | 1 | 1 | 1 | 0 | 1 | 1 | 1 | 0 | 1 | 1 |
| 150 | 1 | 1 | 1 | 1 | 0 | 0 | 1 | 1 | 0 | 1 | 0 | 1 | 1 | 1 | 1 | 0 | 1 | 1 | 0 | 1 | 1 |
| 151 | 1 | 1 | 1 | 1 | 0 | 0 | 1 | 1 | 0 | 1 | 0 | 1 | 1 | 1 | 1 | 1 | 0 | 1 | 0 | 1 | 1 |
| 152 | 1 | 1 | 1 | 1 | 0 | 0 | 1 | 1 | 0 | 1 | 1 | 0 | 0 | 1 | 0 | 0 | 1 | 1 | 0 | 1 | 1 |
| 153 | 1 | 1 | 1 | 1 | 0 | 0 | 1 | 1 | 0 | 1 | 1 | 0 | 0 | 1 | 0 | 1 | 0 | 1 | 0 | 1 | 1 |
| 154 | 1 | 1 | 1 | 1 | 0 | 0 | 1 | 1 | 0 | 1 | 1 | 0 | 0 | 1 | 1 | 0 | 0 | 1 | 0 | 1 | 1 |
| 155 | 1 | 1 | 1 | 1 | 0 | 0 | 1 | 1 | 0 | 1 | 1 | 0 | 0 | 1 | 1 | 1 | 1 | 1 | 0 | 1 | 1 |
| 156 | 1 | 1 | 1 | 1 | 0 | 0 | 1 | 1 | 0 | 1 | 1 | 0 | 1 | 0 | 0 | 1 | 0 | 1 | 0 | 1 | 1 |
| 157 | 1 | 1 | 1 | 1 | 0 | 0 | 1 | 1 | 0 | 1 | 1 | 0 | 1 | 0 | 1 | 0 | 0 | 1 | 0 | 1 | 1 |
| 158 | 1 | 1 | 1 | 1 | 0 | 0 | 1 | 1 | 0 | 1 | 1 | 0 | 1 | 0 | 1 | 1 | 1 | 1 | 0 | 1 | 1 |
| 159 | 1 | 1 | 1 | 1 | 0 | 0 | 1 | 1 | 0 | 1 | 1 | 0 | 1 | 1 | 0 | 1 | 1 | 1 | 0 | 1 | 1 |
| 160 | 1 | 1 | 1 | 1 | 0 | 0 | 1 | 1 | 0 | 1 | 1 | 0 | 1 | 1 | 1 | 0 | 1 | 1 | 0 | 1 | 1 |
| 161 | 1 | 1 | 1 | 1 | 0 | 0 | 1 | 1 | 0 | 1 | 1 | 0 | 1 | 1 | 1 | 1 | 0 | 1 | 0 | 1 | 1 |
| 162 | 1 | 1 | 1 | 1 | 0 | 0 | 1 | 1 | 0 | 1 | 1 | 1 | 0 | 0 | 1 | 0 | 0 | 1 | 0 | 1 | 1 |
| 163 | 1 | 1 | 1 | 1 | 0 | 0 | 1 | 1 | 0 | 1 | 1 | 1 | 0 | 0 | 1 | 1 | 1 | 1 | 0 | 1 | 1 |
| 164 | 1 | 1 | 1 | 1 | 0 | 0 | 1 | 1 | 0 | 1 | 1 | 1 | 0 | 1 | 0 | 1 | 1 | 1 | 0 | 1 | 1 |
| 165 | 1 | 1 | 1 | 1 | 0 | 0 | 1 | 1 | 0 | 1 | 1 | 1 | 0 | 1 | 1 | 0 | 1 | 1 | 0 | 1 | 1 |
| 166 | 1 | 1 | 1 | 1 | 0 | 0 | 1 | 1 | 0 | 1 | 1 | 1 | 0 | 1 | 1 | 1 | 0 | 1 | 0 | 1 | 1 |
| 167 | 1 | 1 | 1 | 1 | 0 | 0 | 1 | 1 | 0 | 1 | 1 | 1 | 1 | 0 | 0 | 1 | 1 | 1 | 0 | 1 | 1 |
| 168 | 1 | 1 | 1 | 1 | 0 | 0 | 1 | 1 | 0 | 1 | 1 | 1 | 1 | 0 | 1 | 0 | 1 | 1 | 0 | 1 | 1 |
| 169 | 1 | 1 | 1 | 1 | 0 | 0 | 1 | 1 | 0 | 1 | 1 | 1 | 1 | 0 | 1 | 1 | 0 | 1 | 0 | 1 | 1 |
| 170 | 1 | 1 | 1 | 1 | 0 | 0 | 1 | 1 | 0 | 1 | 1 | 1 | 1 | 1 | 0 | 0 | 1 | 1 | 0 | 1 | 1 |
| 171 | 1 | 1 | 1 | 1 | 0 | 0 | 1 | 1 | 0 | 1 | 1 | 1 | 1 | 1 | 0 | 1 | 0 | 1 | 0 | 1 | 1 |
| 172 | 1 | 1 | 1 | 1 | 0 | 0 | 1 | 1 | 0 | 1 | 1 | 1 | 1 | 1 | 1 | 0 | 0 | 1 | 0 | 1 | 1 |
| 173 | 1 | 1 | 1 | 1 | 0 | 0 | 1 | 1 | 1 | 0 | 0 | 1 | 0 | 0 | 1 | 0 | 1 | 1 | 0 | 1 | 1 |
| 174 | 1 | 1 | 1 | 1 | 0 | 0 | 1 | 1 | 1 | 0 | 0 | 1 | 0 | 0 | 1 | 1 | 0 | 1 | 0 | 1 | 1 |
| 175 | 1 | 1 | 1 | 1 | 0 | 0 | 1 | 1 | 1 | 0 | 0 | 1 | 0 | 1 | 0 | 0 | 1 | 1 | 0 | 1 | 1 |
| 176 | 1 | 1 | 1 | 1 | 0 | 0 | 1 | 1 | 1 | 0 | 0 | 1 | 0 | 1 | 0 | 1 | 0 | 1 | 0 | 1 | 1 |
| 177 | 1 | 1 | 1 | 1 | 0 | 0 | 1 | 1 | 1 | 0 | 0 | 1 | 0 | 1 | 1 | 0 | 0 | 1 | 0 | 1 | 1 |
| 178 | 1 | 1 | 1 | 1 | 0 | 0 | 1 | 1 | 1 | 0 | 0 | 1 | 0 | 1 | 1 | 1 | 1 | 1 | 0 | 1 | 1 |
| 179 | 1 | 1 | 1 | 1 | 0 | 0 | 1 | 1 | 1 | 0 | 0 | 1 | 1 | 0 | 0 | 1 | 0 | 1 | 0 | 1 | 1 |
| 180 | 1 | 1 | 1 | 1 | 0 | 0 | 1 | 1 | 1 | 0 | 0 | 1 | 1 | 0 | 1 | 0 | 0 | 1 | 0 | 1 | 1 |
| 181 | 1 | 1 | 1 | 1 | 0 | 0 | 1 | 1 | 1 | 0 | 0 | 1 | 1 | 0 | 1 | 1 | 1 | 1 | 0 | 1 | 1 |
| 182 | 1 | 1 | 1 | 1 | 0 | 0 | 1 | 1 | 1 | 0 | 0 | 1 | 1 | 1 | 0 | 1 | 1 | 1 | 0 | 1 | 1 |
| 183 | 1 | 1 | 1 | 1 | 0 | 0 | 1 | 1 | 1 | 0 | 0 | 1 | 1 | 1 | 1 | 0 | 1 | 1 | 0 | 1 | 1 |
| 184 | 1 | 1 | 1 | 1 | 0 | 0 | 1 | 1 | 1 | 0 | 0 | 1 | 1 | 1 | 1 | 1 | 0 | 1 | 0 | 1 | 1 |
| 185 | 1 | 1 | 1 | 1 | 0 | 0 | 1 | 1 | 1 | 0 | 1 | 0 | 0 | 1 | 0 | 0 | 1 | 1 | 0 | 1 | 1 |
| 186 | 1 | 1 | 1 | 1 | 0 | 0 | 1 | 1 | 1 | 0 | 1 | 0 | 0 | 1 | 0 | 1 | 0 | 1 | 0 | 1 | 1 |
| 187 | 1 | 1 | 1 | 1 | 0 | 0 | 1 | 1 | 1 | 0 | 1 | 0 | 0 | 1 | 1 | 0 | 0 | 1 | 0 | 1 | 1 |
| 188 | 1 | 1 | 1 | 1 | 0 | 0 | 1 | 1 | 1 | 0 | 1 | 0 | 0 | 1 | 1 | 1 | 1 | 1 | 0 | 1 | 1 |
| 189 | 1 | 1 | 1 | 1 | 0 | 0 | 1 | 1 | 1 | 0 | 1 | 0 | 1 | 0 | 0 | 1 | 0 | 1 | 0 | 1 | 1 |
| 190 | 1 | 1 | 1 | 1 | 0 | 0 | 1 | 1 | 1 | 0 | 1 | 0 | 1 | 0 | 1 | 0 | 0 | 1 | 0 | 1 | 1 |
| 191 | 1 | 1 | 1 | 1 | 0 | 0 | 1 | 1 | 1 | 0 | 1 | 0 | 1 | 0 | 1 | 1 | 1 | 1 | 0 | 1 | 1 |
| 192 | 1 | 1 | 1 | 1 | 0 | 0 | 1 | 1 | 1 | 0 | 1 | 0 | 1 | 1 | 0 | 1 | 1 | 1 | 0 | 1 | 1 |
| 193 | 1 | 1 | 1 | 1 | 0 | 0 | 1 | 1 | 1 | 0 | 1 | 0 | 1 | 1 | 1 | 0 | 1 | 1 | 0 | 1 | 1 |
| 194 | 1 | 1 | 1 | 1 | 0 | 0 | 1 | 1 | 1 | 0 | 1 | 0 | 1 | 1 | 1 | 1 | 0 | 1 | 0 | 1 | 1 |
| 195 | 1 | 1 | 1 | 1 | 0 | 0 | 1 | 1 | 1 | 0 | 1 | 1 | 0 | 0 | 1 | 0 | 0 | 1 | 0 | 1 | 1 |
| 196 | 1 | 1 | 1 | 1 | 0 | 0 | 1 | 1 | 1 | 0 | 1 | 1 | 0 | 0 | 1 | 1 | 1 | 1 | 0 | 1 | 1 |
| 197 | 1 | 1 | 1 | 1 | 0 | 0 | 1 | 1 | 1 | 0 | 1 | 1 | 0 | 1 | 0 | 1 | 1 | 1 | 0 | 1 | 1 |
| 198 | 1 | 1 | 1 | 1 | 0 | 0 | 1 | 1 | 1 | 0 | 1 | 1 | 0 | 1 | 1 | 0 | 1 | 1 | 0 | 1 | 1 |
| 199 | 1 | 1 | 1 | 1 | 0 | 0 | 1 | 1 | 1 | 0 | 1 | 1 | 0 | 1 | 1 | 1 | 0 | 1 | 0 | 1 | 1 |
| 200 | 1 | 1 | 1 | 1 | 0 | 0 | 1 | 1 | 1 | 0 | 1 | 1 | 1 | 0 | 0 | 1 | 1 | 1 | 0 | 1 | 1 |
| 201 | 1 | 1 | 1 | 1 | 0 | 0 | 1 | 1 | 1 | 0 | 1 | 1 | 1 | 0 | 1 | 0 | 1 | 1 | 0 | 1 | 1 |
| 202 | 1 | 1 | 1 | 1 | 0 | 0 | 1 | 1 | 1 | 0 | 1 | 1 | 1 | 0 | 1 | 1 | 0 | 1 | 0 | 1 | 1 |
| 203 | 1 | 1 | 1 | 1 | 0 | 0 | 1 | 1 | 1 | 0 | 1 | 1 | 1 | 1 | 0 | 0 | 1 | 1 | 0 | 1 | 1 |
| 204 | 1 | 1 | 1 | 1 | 0 | 0 | 1 | 1 | 1 | 0 | 1 | 1 | 1 | 1 | 0 | 1 | 0 | 1 | 0 | 1 | 1 |
| 205 | 1 | 1 | 1 | 1 | 0 | 0 | 1 | 1 | 1 | 0 | 1 | 1 | 1 | 1 | 1 | 0 | 0 | 1 | 0 | 1 | 1 |
| 206 | 1 | 1 | 1 | 1 | 0 | 0 | 1 | 1 | 1 | 1 | 0 | 0 | 1 | 0 | 0 | 1 | 0 | 1 | 0 | 1 | 1 |
| 207 | 1 | 1 | 1 | 1 | 0 | 0 | 1 | 1 | 1 | 1 | 0 | 0 | 1 | 0 | 1 | 0 | 0 | 1 | 0 | 1 | 1 |
| 208 | 1 | 1 | 1 | 1 | 0 | 0 | 1 | 1 | 1 | 1 | 0 | 0 | 1 | 0 | 1 | 1 | 1 | 1 | 0 | 1 | 1 |
| 209 | 1 | 1 | 1 | 1 | 0 | 0 | 1 | 1 | 1 | 1 | 0 | 0 | 1 | 1 | 0 | 1 | 1 | 1 | 0 | 1 | 1 |
| 210 | 1 | 1 | 1 | 1 | 0 | 0 | 1 | 1 | 1 | 1 | 0 | 0 | 1 | 1 | 1 | 0 | 1 | 1 | 0 | 1 | 1 |
| 211 | 1 | 1 | 1 | 1 | 0 | 0 | 1 | 1 | 1 | 1 | 0 | 0 | 1 | 1 | 1 | 1 | 0 | 1 | 0 | 1 | 1 |
| 212 | 1 | 1 | 1 | 1 | 0 | 0 | 1 | 1 | 1 | 1 | 0 | 1 | 0 | 0 | 1 | 0 | 0 | 1 | 0 | 1 | 1 |
| 213 | 1 | 1 | 1 | 1 | 0 | 0 | 1 | 1 | 1 | 1 | 0 | 1 | 0 | 0 | 1 | 1 | 1 | 1 | 0 | 1 | 1 |
| 214 | 1 | 1 | 1 | 1 | 0 | 0 | 1 | 1 | 1 | 1 | 0 | 1 | 0 | 1 | 0 | 1 | 1 | 1 | 0 | 1 | 1 |
| 215 | 1 | 1 | 1 | 1 | 0 | 0 | 1 | 1 | 1 | 1 | 0 | 1 | 0 | 1 | 1 | 0 | 1 | 1 | 0 | 1 | 1 |
| 216 | 1 | 1 | 1 | 1 | 0 | 0 | 1 | 1 | 1 | 1 | 0 | 1 | 0 | 1 | 1 | 1 | 0 | 1 | 0 | 1 | 1 |
| 217 | 1 | 1 | 1 | 1 | 0 | 0 | 1 | 1 | 1 | 1 | 0 | 1 | 1 | 0 | 0 | 1 | 1 | 1 | 0 | 1 | 1 |
| 218 | 1 | 1 | 1 | 1 | 0 | 0 | 1 | 1 | 1 | 1 | 0 | 1 | 1 | 0 | 1 | 0 | 1 | 1 | 0 | 1 | 1 |
| 219 | 1 | 1 | 1 | 1 | 0 | 0 | 1 | 1 | 1 | 1 | 0 | 1 | 1 | 0 | 1 | 1 | 0 | 1 | 0 | 1 | 1 |
| 220 | 1 | 1 | 1 | 1 | 0 | 0 | 1 | 1 | 1 | 1 | 0 | 1 | 1 | 1 | 0 | 0 | 1 | 1 | 0 | 1 | 1 |
| 221 | 1 | 1 | 1 | 1 | 0 | 0 | 1 | 1 | 1 | 1 | 0 | 1 | 1 | 1 | 0 | 1 | 0 | 1 | 0 | 1 | 1 |
| 222 | 1 | 1 | 1 | 1 | 0 | 0 | 1 | 1 | 1 | 1 | 0 | 1 | 1 | 1 | 1 | 0 | 0 | 1 | 0 | 1 | 1 |
| 223 | 1 | 1 | 1 | 1 | 0 | 0 | 1 | 1 | 1 | 1 | 1 | 0 | 0 | 1 | 0 | 1 | 1 | 1 | 0 | 1 | 1 |
| 224 | 1 | 1 | 1 | 1 | 0 | 0 | 1 | 1 | 1 | 1 | 1 | 0 | 0 | 1 | 1 | 0 | 1 | 1 | 0 | 1 | 1 |
| 225 | 1 | 1 | 1 | 1 | 0 | 0 | 1 | 1 | 1 | 1 | 1 | 0 | 0 | 1 | 1 | 1 | 0 | 1 | 0 | 1 | 1 |
| 226 | 1 | 1 | 1 | 1 | 0 | 0 | 1 | 1 | 1 | 1 | 1 | 0 | 1 | 0 | 0 | 1 | 1 | 1 | 0 | 1 | 1 |
| 227 | 1 | 1 | 1 | 1 | 0 | 0 | 1 | 1 | 1 | 1 | 1 | 0 | 1 | 0 | 1 | 0 | 1 | 1 | 0 | 1 | 1 |
| 228 | 1 | 1 | 1 | 1 | 0 | 0 | 1 | 1 | 1 | 1 | 1 | 0 | 1 | 0 | 1 | 1 | 0 | 1 | 0 | 1 | 1 |
| 229 | 1 | 1 | 1 | 1 | 0 | 0 | 1 | 1 | 1 | 1 | 1 | 0 | 1 | 1 | 0 | 0 | 1 | 1 | 0 | 1 | 1 |
| 230 | 1 | 1 | 1 | 1 | 0 | 0 | 1 | 1 | 1 | 1 | 1 | 0 | 1 | 1 | 0 | 1 | 0 | 1 | 0 | 1 | 1 |
| 231 | 1 | 1 | 1 | 1 | 0 | 0 | 1 | 1 | 1 | 1 | 1 | 0 | 1 | 1 | 1 | 0 | 0 | 1 | 0 | 1 | 1 |
| 232 | 1 | 1 | 1 | 1 | 0 | 0 | 1 | 1 | 1 | 1 | 1 | 1 | 0 | 0 | 1 | 0 | 1 | 1 | 0 | 1 | 1 |
| 233 | 1 | 1 | 1 | 1 | 0 | 0 | 1 | 1 | 1 | 1 | 1 | 1 | 0 | 0 | 1 | 1 | 0 | 1 | 0 | 1 | 1 |
| 234 | 1 | 1 | 1 | 1 | 0 | 0 | 1 | 1 | 1 | 1 | 1 | 1 | 0 | 1 | 0 | 0 | 1 | 1 | 0 | 1 | 1 |
| 235 | 1 | 1 | 1 | 1 | 0 | 0 | 1 | 1 | 1 | 1 | 1 | 1 | 0 | 1 | 0 | 1 | 0 | 1 | 0 | 1 | 1 |
| 236 | 1 | 1 | 1 | 1 | 0 | 0 | 1 | 1 | 1 | 1 | 1 | 1 | 0 | 1 | 1 | 0 | 0 | 1 | 0 | 1 | 1 |
| 237 | 1 | 1 | 1 | 1 | 0 | 0 | 1 | 1 | 1 | 1 | 1 | 1 | 1 | 0 | 0 | 1 | 0 | 1 | 0 | 1 | 1 |
| 238 | 1 | 1 | 1 | 1 | 0 | 0 | 1 | 1 | 1 | 1 | 1 | 1 | 1 | 0 | 1 | 0 | 0 | 1 | 0 | 1 | 1 |

### Example 2—Generation and Selection of a Larger Barcode Set

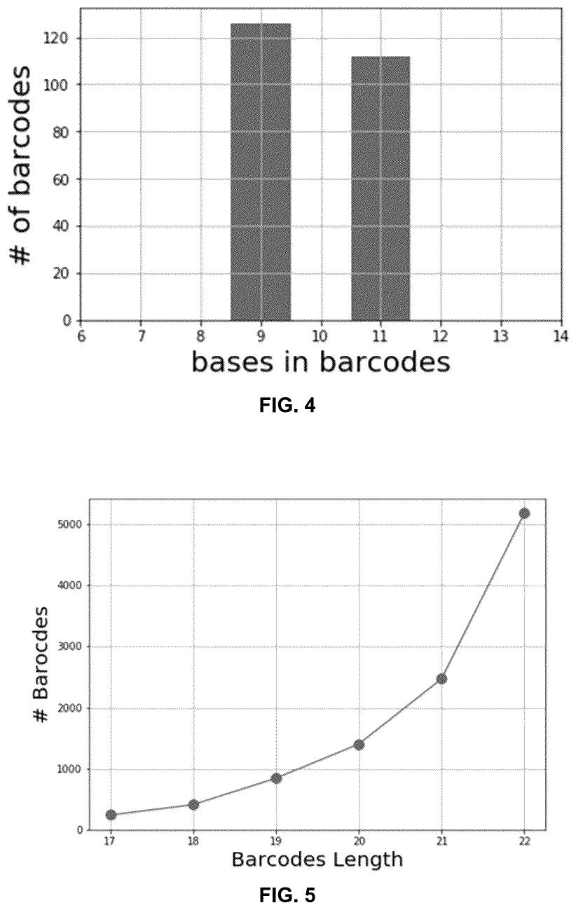

Generating a larger number of barcodes (e.g., more than the 238 barcodes generated in Example 1) may require an increase in the acceptable barcode length in base space, and hence in flow space (e.g., as shown in FIG. 5). In generating a larger barcode set, it may also be beneficial to improve distinction among barcode sequences by increasing the effective edit-distance between each pair of barcode (e.g., from the minimum edit distance of 2 in Example 1 to a minimum edit distance of at least 4 as described here). In some embodiments, the effective-edit distance is at least 4, at least 5, at least 6, at least 7, at least 8, at least 9, at least 10, at least 11, at least 12, at least 13, at least 14, or at least 15. The flow sequence used in this example is TGCA. The requirements (e.g., filters and constraints) for generating a larger barcode set (e.g., more than 1000 distinct barcode sequences) included the increased barcode length, increased edit distance, and constraints on H-mer number and size.

Barcodes were determined for an effective length of 29 flows. The barcode sequences included the following regions: preamble (4 flows, 4 bases), constant prefix (3 flows 1 base), variable sequence, and constant post sequence (4 flows, 3 bases). As in Example 1, the preamble consisted of 4 nucleotides (TGCA) and accounted for 4 flows. Each barcode sequence then started with a C (e.g., the constant prefix, or the sample data identification sequence as described in Example 1). Thus, in accordance with the TGCA flow order, the flowspace vector for each barcode in this set begins as: [1,1,1,1,0,0,1 . . . ] (see Table 4 below). Following the constant prefix, the barcode variable sequence is allotted 18 flows (where the variable sequence length in base space is not constant). The constant post sequence is GAT.

In addition, barcodes were required to have an effective edit distance of at least 4 from each other (e.g., there was a minimum edit distance of at least 4 between each possible pair of barcodes in the set). In effect, this minimum edit distance is only calculated for the variable sequence portions of each barcode sequence (e.g., because the preamble, constant prefix, and constant post sequences are identical for each barcode in the set). Further, each of the values in flow space for the variable sequence regions was set to 0, 1, or 2 (e.g., there were no homopolymers that are longer than 2 nucleotides long in base space). For each barcode, only one value in flow space was 2 (e.g., no more than one 2-mer was allowed per barcode, and each barcode was required to have one 2-mer). Following these requirements, the barcode variable sequences may be either 11 bases or 13 bases in length.

These requirements result in a set of barcodes where, for each pair of barcodes, most sequence differences between the vectors representing the barcodes (see e.g., the flowspace values in Table 4 below) may be either from a 0 to a 1 or from a 1 to a 0. Few of the sequences differences may be from a 1 to a 2 or from a 2 to a 1. All barcodes have a constant length in flow space, as described above for Example 1. The constant length in flow space may lead to each of the barcodes having similar but not exact length in base space, where the differences may come from the length differences of the variable sequences). The overall length of each barcode in the set is either 19 or 21 bases. These parameters serve to increase the contribution of context to signal difference.

In this example, the sequence of interest (or “template polynucleotide”) can be located after the T of flow number 28, which ends each of these barcode sequences (e.g., the end of the constant post sequence GAT). Following the parameters described above, the selection resulted in 1018 distinct barcode sequences. A subset of these barcodes is displayed in Table 4, illustrating the correspondence between flow space and base space. Sequence ID numbers for all the barcode sequences that satisfy the above criteria are also provided in Table 5.

TABLE 4
List of 4 example barcode sequences (SEQ ID NOs: 283, 250, 332
and 400) and the resultant flowspace values for 29 flows.

| SEQ ID | 0 | 1 | 2 | 3 | 4 | 5 | 6 | 7 | 8 | 9 | 10 | 11 | 12 | 13 | 14 |
|---|---|---|---|---|---|---|---|---|---|---|---|---|---|---|---|
| NO: | T | G | C | A | T | G | C | A | T | G | C | A | T | G | C |
| 283 | 1 | 1 | 1 | 1 | 0 | 0 | 1 | 0 | 0 | 1 | 0 | 1 | 1 | 0 | 0 |
| 250 | 1 | 1 | 1 | 1 | 0 | 0 | 1 | 0 | 0 | 1 | 0 | 0 | 1 | 2 | 0 |
| 332 | 1 | 1 | 1 | 1 | 0 | 0 | 1 | 0 | 0 | 1 | 1 | 0 | 2 | 0 | 1 |
| 365 | 1 | 1 | 1 | 1 | 0 | 0 | 1 | 0 | 0 | 1 | 1 | 1 | 0 | 1 | 0 |
| 400 | 1 | 1 | 1 | 1 | 0 | 0 | 1 | 0 | 0 | 1 | 1 | 1 | 1 | 2 | 1 |

| SEQ ID | 15 | 16 | 17 | 18 | 19 | 20 | 21 | 22 | 23 | 24 | 25 | 26 | 27 | 28 |
|---|---|---|---|---|---|---|---|---|---|---|---|---|---|---|
| NO: | A | T | G | C | A | T | G | C | A | T | G | C | A | T |
| 283 | 1 | 2 | 0 | 1 | 0 | 0 | 1 | 1 | 1 | 1 | 1 | 0 | 1 | 1 |
| 250 | 1 | 1 | 0 | 1 | 0 | 1 | 1 | 0 | 1 | 1 | 1 | 0 | 1 | 1 |
| 332 | 0 | 0 | 1 | 1 | 0 | 0 | 1 | 1 | 1 | 1 | 1 | 0 | 1 | 1 |
| 365 | 0 | 1 | 0 | 1 | 1 | 1 | 0 | 2 | 1 | 0 | 1 | 0 | 1 | 1 |
| 400 | 0 | 0 | 1 | 1 | 0 | 0 | 1 | 0 | 1 | 0 | 1 | 0 | 1 | 1 |

### List of Barcode Sequences

Provided herein in Table 5 is a list of barcode sequences generated using the methods described herein, and as described in Example 2 above.

TABLE 5
List of barcode sequences resultant from
29 flow cycles as described in Example 2.

| Sequence | SEQ ID NO: |
|---|---|
| TGCACGGTACATGCATGAT | 239 |
| TGCACGTAATGCTCATGAT | 240 |
| TGCACGTATGGCAGCTGAT | 241 |
| TGCACGTCGCATTCATGAT | 242 |
| TGCACGTCTGATGCCAGAT | 243 |
| TGCACGGTCAGCATGTGAT | 244 |
| TGCACGTCCATCATATGAT | 245 |
| TGCACGTCATTGCACAGAT | 246 |
| TGCACGTGTGCAACATGAT | 247 |
| TGCACGTGTGCATGGCGAT | 248 |
| TGCACGGTGAGCAGATGAT | 249 |
| TGCACGTGGATCTGATGAT | 250 |
| TGCACGTGATTGATGCATGAT | 251 |
| TGCACGTGCGCAAGCAGAT | 252 |
| TGCACGTGCTCATGGCATGAT | 253 |
| TGCACGGTGCTGCTATGAT | 254 |
| TGCACGTGGCACACATGAT | 255 |
| TGCACGTGCAACATGAGAT | 256 |
| TGCACGTGCAGTTCATGAT | 257 |
| TGCACGTGCATAGCCTGAT | 258 |
| TGCACGGTGCATATCAGAT | 259 |
| TGCACGTGGCATGTGTGAT | 260 |
| TGCACGTGCAATGCGCATGAT | 261 |
| TGCACGTGCATGGCATCTGAT | 262 |
| TGCACGACTCATGCCTGAT | 263 |
| TGCACGGACAGCTGCAGAT | 264 |
| TGCACGACCATATCATGAT | 265 |
| TGCACGACATTGTGCTGAT | 266 |
| TGCACGACATGAAGCAGAT | 267 |
| TGCACGACATGCACCTGAT | 268 |
| TGCACGACATGCATGAATGAT | 269 |
| TGCACGGAGTGCATGCATGAT | 270 |
| TGCACGAGGAGCATGTGAT | 271 |
| TGCACGAGATTGAGATGAT | 272 |
| TGCACGAGATGCCTCAGAT | 273 |
| TGCACGAGCGCTCAATGAT | 274 |
| TGCACGGAGCTATGCAGAT | 275 |
| TGCACGAGGCTGATCTGAT | 276 |
| TGCACGAGCTTGCTGTGAT | 277 |
| TGCACGAGCACAATGCATGAT | 278 |
| TGCACGAGCATCAGGTGAT | 279 |
| TGCACGAGCATGCATGGCGAT | 280 |
| TGCACGGATAGATGCTGAT | 281 |
| TGCACGATTAGCATATGAT | 282 |
| TGCACGATATTCGCATGAT | 283 |
| TGCACGATATCAATCAGAT | 284 |
| TGCACGATATCATGGTGAT | 285 |
| TGCACGGATCGCATGCGAT | 286 |
| TGCACGATTCTGTCATGAT | 287 |
| TGCACGATCTTGAGCTGAT | 288 |
| TGCACGATCACAAGCTGAT | 289 |
| TGCACGATCATATGGCGAT | 290 |
| TGCACGGATCATGCGTGAT | 291 |
| TGCACGATTCATGCTCGAT | 292 |
| TGCACGATGTTGAGCAGAT | 293 |
| TGCACGATGTGCCATAGAT | 294 |
| TGCACGATGACTGCCAGAT | 295 |
| TGCACGGATGAGCACTGAT | 296 |
| TGCACGATTGATGCGCGAT | 297 |
| TGCACGATGCCGTGCAGAT | 298 |
| TGCACGATGCGAATATGAT | 299 |
| TGCACGATGCTCTGGCGAT | 300 |
| TGCACGGATGCTCACTGAT | 301 |
| TGCACGATTGCTGCAGATGAT | 302 |
| TGCACGATGCCACTGTGAT | 303 |
| TGCACGATGCAGGAGAGAT | 304 |
| TGCACGATGCAGATTCGAT | 305 |
| TGCACGGATGCAGCTAGAT | 306 |
| TGCACGATTGCATATGATGAT | 307 |
| TGCACGATGCCATCTCATGAT | 308 |
| TGCACGATGCATTCAGCAGAT | 309 |
| TGCACGATGCATGAACATGAT | 310 |
| TGCACGGCGTGCGCATGAT | 311 |
| TGCACGCGGAGCATCAGAT | 312 |
| TGCACGCGATTCATGTGAT | 313 |
| TGCACGCGATGCCTCTGAT | 314 |
| TGCACGCGCGCAGAATGAT | 315 |
| TGCACGGCGCTGAGCTGAT | 316 |
| TGCACGCGGCTGATATGAT | 317 |
| TGCACGCGCTTGCATGCAGAT | 318 |
| TGCACGCGCACAATATGAT | 319 |
| TGCACGCGCAGATGGCATGAT | 320 |
| TGCACGGCGCAGCTGTGAT | 321 |
| TGCACGCGGCATACATGAT | 322 |
| TGCACGCGCAATATGCGAT | 323 |
| TGCACGCGCATCCTGCATGAT | 324 |
| TGCACGCGCATGCTTAGAT | 325 |
| TGCACGGCTAGATGCAGAT | 326 |
| TGCACGCTTAGCTGCTGAT | 327 |
| TGCACGCTATTCACATGAT | 328 |
| TGCACGCTATGAATATGAT | 329 |
| TGCACGCTATGCGAATGAT | 330 |
| TGCACGGCTATGCATCGAT | 331 |
| TGCACGCTTCGCGCATGAT | 332 |
| TGCACGCTCGGCATGAGAT | 333 |
| TGCACGCTCTGTTGATGAT | 334 |
| TGCACGCTCTGCACCTGAT | 335 |
| TGCACGGCTCACTCATGAT | 336 |
| TGCACGCTTCACAGATGAT | 337 |
| TGCACGCTCAACATGCGAT | 338 |
| TGCACGCTCAGAAGCTGAT | 339 |
| TGCACGCTCATATGGCATGAT | 340 |
| TGCACGGCTCATCGCTGAT | 341 |
| TGCACGCTTCATGCTGCAGAT | 342 |
| TGCACGCTGTTGCATGATGAT | 343 |
| TGCACGCTGAGAATCTGAT | 344 |
| TGCACGCTGATCAGGCGAT | 345 |
| TGCACGGCTGATCATAGAT | 346 |
| TGCACGCTTGCGATGTGAT | 347 |
| TGCACGCTGCCTGCTGCTGAT | 348 |
| TGCACGCTGCAGGCACGAT | 349 |
| TGCACGCTGCATGAAGATGAT | 350 |
| TGCACGGCACGATGCTGAT | 351 |
| TGCACGCAACGCATATGAT | 352 |
| TGCACGCACTTCGCATGAT | 353 |
| TGCACGCACTGTTGCAGAT | 354 |
| TGCACGCACTGCTCCTGAT | 355 |
| TGCACGGCACTGCAGTGAT | 356 |
| TGCACGCAACACATGTGAT | 357 |
| TGCACGCACAAGTCATGAT | 358 |
| TGCACGCACAGCCAGCATGAT | 359 |
| TGCACGCACATAGCCTGAT | 360 |
| TGCACGGCACATATGAGAT | 361 |
| TGCACGCAACATCATCGAT | 362 |
| TGCACGCACAATGCGAGAT | 363 |
| TGCACGCAGTCAAGCTGAT | 364 |
| TGCACGCAGTCATCCAGAT | 365 |
| TGCACGCAGAGCTGCAATGAT | 366 |
| TGCACGGCAGAGCAGCGAT | 367 |
| TGCACGCAAGATATGCATGAT | 368 |
| TGCACGCAGAATGCGTGAT | 369 |
| TGCACGCAGATGGCACATGAT | 370 |
| TGCACGCAGATGCAATGAGAT | 371 |
| TGCACGCAGCTCATGAATGAT | 372 |
| TGCACGGCAGCAGACTGAT | 373 |
| TGCACGCAAGCATGTCGAT | 374 |
| TGCACGCAGCCATGTGATGAT | 375 |
| TGCACGCATACTTGATGAT | 376 |
| TGCACGCATACATCCTGAT | 377 |
| TGCACGGCATAGAGATGAT | 378 |
| TGCACGCAATAGCTCAGAT | 379 |
| TGCACGCATAATGTCTGAT | 380 |
| TGCACGCATATGGCAGCAGAT | 381 |
| TGCACGCATCGAGCCAGAT | 382 |
| TGCACGGCATCGCTGTGAT | 383 |
| TGCACGCAATCTATCTGAT | 384 |
| TGCACGCATCCTCACAGAT | 385 |
| TGCACGCATCTGGCGCGAT | 386 |
| TGCACGCATCACATTAGAT | 387 |
| TGCACGGCATCAGTGAGAT | 388 |
| TGCACGCAATCATGATCAGAT | 389 |
| TGCACGCATCCATGATGTGAT | 390 |
| TGCACGCATCATTGCTATGAT | 391 |
| TGCACGCATGTATGGCGAT | 392 |
| TGCACGGCATGTCTGTGAT | 393 |
| TGCACGCAATGTGTGCATGAT | 394 |
| TGCACGCATGGTGACTGAT | 395 |
| TGCACGCATGTGGCTCGAT | 396 |
| TGCACGCATGACACCAGAT | 397 |
| TGCACGGCATGACAGTGAT | 398 |
| TGCACGCAATGAGTGTGAT | 399 |
| TGCACGCATGGCGCGAGAT | 400 |
| TGCACGCATGCGGCACATGAT | 401 |
| TGCACGCATGCTAGGTGAT | 402 |
| TGCACGGCATGCTGTAGAT | 403 |
| TGCACGCAATGCACGCATGAT | 404 |
| TGCACGCATGGCAGCTCTGAT | 405 |
| TGCACGCATGCAATACGAT | 406 |
| TGCACGCATGCATCCTGAGAT | 407 |
| TGCACTTACGCATCATGAT | 408 |
| TGCACTACCTGATGCAGAT | 409 |
| TGCACTACTGGCAGCTGAT | 410 |
| TGCACTACAGCAATGTGAT | 411 |
| TGCACTACATCATGGCATGAT | 412 |
| TGCACTTACATGTCATGAT | 413 |
| TGCACTACCATGCTGAGAT | 414 |
| TGCACTACATTGCACAGAT | 415 |
| TGCACTAGTGCAACATGAT | 416 |
| TGCACTAGTGCATGGTGAT | 417 |
| TGCACTAGAGCATGCAATGAT | 418 |
| TGCACTTAGATATCATGAT | 419 |
| TGCACTAGGATCATGCGAT | 420 |
| TGCACTAGATTGCGCAGAT | 421 |
| TGCACTAGCGCAATGAGAT | 422 |
| TGCACTAGCTCAGCCAGAT | 423 |
| TGCACTTAGCTGTGATGAT | 424 |
| TGCACTAGGCACTGCAGAT | 425 |
| TGCACTAGCAAGATCAGAT | 426 |
| TGCACTAGCAGCCAGCGAT | 427 |
| TGCACTAGCATCTGGTGAT | 428 |
| TGCACTTAGCATCACTGAT | 429 |
| TGCACTAGGCATGAGTGAT | 430 |
| TGCACTAGCAATGCATATGAT | 431 |
| TGCACTATAGCAATGCGAT | 432 |
| TGCACTATATAGCAATGAT | 433 |
| TGCACTTATATCTCATGAT | 434 |
| TGCACTATTATGATATGAT | 435 |
| TGCACTATATTGCGATGAT | 436 |
| TGCACTATCGCTTGATGAT | 437 |
| TGCACTATCTCATAATGAT | 438 |
| TGCACTTATCTGATCTGAT | 439 |
| TGCACTATTCAGATGCATGAT | 440 |
| TGCACTATCAAGCTCAGAT | 441 |
| TGCACTATCATCCAGTGAT | 442 |
| TGCACTATCATGTGGTGAT | 443 |
| TGCACTTATCATGCGCGAT | 444 |
| TGCACTATTGTATGCTGAT | 445 |
| TGCACTATGTTGTGCAGAT | 446 |
| TGCACTATGTGCCAGAGAT | 447 |
| TGCACTATGTGCATTCGAT | 448 |
| TGCACTTATGAGCGCTGAT | 449 |
| TGCACTATTGATATGAGAT | 450 |
| TGCACTATGAATGAGCGAT | 451 |
| TGCACTATGCGAACATGAT | 452 |
| TGCACTATGCGATGGTGAT | 453 |
| TGCACTTATGCTATCAGAT | 454 |
| TGCACTATTGCTCTGCATGAT | 455 |
| TGCACTATGCCACAGCATGAT | 456 |
| TGCACTATGCACCATCGAT | 457 |
| TGCACTATGCAGCGGAGAT | 458 |
| TGCACTTATGCATGTAGAT | 459 |
| TGCACTATTGCATGCTCTGAT | 460 |
| TGCACTCGTGGCATGCATGAT | 461 |
| TGCACTCGAGATTGATGAT | 462 |
| TGCACTCGATGAGCCTGAT | 463 |
| TGCACTTCGATGCTGTGAT | 464 |
| TGCACTCGGCGCGCATGAT | 465 |
| TGCACTCGCGGCATATGAT | 466 |
| TGCACTCGCGCAATGCGAT | 467 |
| TGCACTCGCTGCAGGTGAT | 468 |
| TGCACTTCGCACACATGAT | 469 |
| TGCACTCGGCACATGTGAT | 470 |
| TGCACTCGCAATAGATGAT | 471 |
| TGCACTCGCATCCATGCAGAT | 472 |
| TGCACTCGCATGTGGAGAT | 473 |
| TGCACTCGCATGATCAATGAT | 474 |
| TGCACTTCGCATGCTCGAT | 475 |
| TGCACTCTTAGCATGTGAT | 476 |
| TGCACTCTATTCATATGAT | 477 |
| TGCACTCTATGTTGCTGAT | 478 |
| TGCACTCTATGCGCCAGAT | 479 |
| TGCACTCTCGCATGCAATGAT | 480 |
| TGCACTTCTCTATGATGAT | 481 |
| TGCACTCTTCTCAGCAGAT | 482 |
| TGCACTCTCTTGATGCGAT | 483 |
| TGCACTCTCACAAGCTGAT | 484 |
| TGCACTCTCACATGGAGAT | 485 |
| TGCACTCTCATCTGCAATGAT | 486 |
| TGCACTTCTCATGTCAGAT | 487 |
| TGCACTCTTCATGAGCATGAT | 488 |
| TGCACTCTCAATGCACGAT | 489 |
| TGCACTCTGTATTGCAGAT | 490 |
| TGCACTCTGTCAGCCTGAT | 491 |
| TGCACTTCTGTGAGATGAT | 492 |
| TGCACTCTTGTGATCTGAT | 493 |
| TGCACTCTGTTGCTCAGAT | 494 |
| TGCACTCTGACAATGCATGAT | 495 |
| TGCACTCTGAGTGCCAGAT | 496 |
| TGCACTTCTGATGATAGAT | 497 |
| TGCACTCTTGATGCACATGAT | 498 |
| TGCACTCTGAATGCATGCGAT | 499 |
| TGCACTCTGCTCCTGCGAT | 500 |
| TGCACTCTGCTCATTCATGAT | 501 |
| TGCACTCTGCTGTGCAATGAT | 502 |
| TGCACTTCTGCTGCATGAGAT | 503 |
| TGCACTCTTGCAGAGCGAT | 504 |
| TGCACTCTGCCAGCGTGAT | 505 |
| TGCACTCTGCAGGCTCATGAT | 506 |
| TGCACTCTGCATATTGCTGAT | 507 |
| TGCACTTCACTCATGAGAT | 508 |
| TGCACTCAACTGATATGAT | 509 |
| TGCACTCACTTGCTGTGAT | 510 |
| TGCACTCACAGTTGATGAT | 511 |
| TGCACTCACAGACAATGAT | 512 |
| TGCACTTCACAGCATAGAT | 513 |
| TGCACTCAACATATCTGAT | 514 |
| TGCACTCACAATCGCAGAT | 515 |
| TGCACTCACATGGAGAGAT | 516 |
| TGCACTCACATGCAATGCGAT | 517 |
| TGCACTTCAGTGAGCAGAT | 518 |
| TGCACTCAAGAGCAGTGAT | 519 |
| TGCACTCAGAAGCATCGAT | 520 |
| TGCACTCAGATCCTGCATGAT | 521 |
| TGCACTCAGATGTCCAGAT | 522 |
| TGCACTCAGCGATGCAATGAT | 523 |
| TGCACTTCAGCGCTCTGAT | 524 |
| TGCACTCAAGCGCACAGAT | 525 |
| TGCACTCAGCCTCATGCTGAT | 526 |
| TGCACTCAGCTGGATCGAT | 527 |
| TGCACTCAGCTGCGGCGAT | 528 |
| TGCACTTCAGCATACAGAT | 529 |
| TGCACTCAAGCATGTGCTGAT | 530 |
| TGCACTCAGCCATGCGATGAT | 531 |
| TGCACTCATAGCCTGCATGAT | 532 |
| TGCACTCATATCGCCTGAT | 533 |
| TGCACTTCATATCTGAGAT | 534 |
| TGCACTCAATATGATGCAGAT | 535 |
| TGCACTCATAATGCATCTGAT | 536 |
| TGCACTCATCGCCACTGAT | 537 |
| TGCACTCATCGCAGGAGAT | 538 |
| TGCACTCATCTGCGCAATGAT | 539 |
| TGCACTTCATCTGCATCAGAT | 540 |
| TGCACTCAATCACATCATGAT | 541 |
| TGCACTCATGGTATATGAT | 542 |
| TGCACTCATGTCCACAGAT | 543 |
| TGCACTCATGTGTGGTGAT | 544 |
| TGCACTTCATGACAGAGAT | 545 |
| TGCACTCAATGAGAGCATGAT | 546 |
| TGCACTCATGGAGCATATGAT | 547 |
| TGCACTCATGATTACTGAT | 548 |
| TGCACTCATGATCAATGTGAT | 549 |
| TGCACTTCATGCGTATGAT | 550 |
| TGCACTCAATGCGCTGCAGAT | 551 |
| TGCACTCATGGCTAGCATGAT | 552 |
| TGCACTCATGCAACTGATGAT | 553 |
| TGCACTCATGCAGAATCTGAT | 554 |
| TGCACTCATGCAGATGGAGAT | 555 |
| TGCACTTCATGCATCTCAGAT | 556 |
| TGCACTCAATGCATCAGCGAT | 557 |
| TGCACTGTAGGCATCTGAT | 558 |
| TGCACTGTAGCAATGAGAT | 559 |
| TGCACTGTATGTGCCAGAT | 560 |
| TGCACTTGTATGATGTGAT | 561 |
| TGCACTGTTCGCTGCAGAT | 562 |
| TGCACTGTCGGCAGCTGAT | 563 |
| TGCACTGTCTGAATATGAT | 564 |
| TGCACTGTCTGCTGGTGAT | 565 |
| TGCACTTGTCACGCATGAT | 566 |
| TGCACTGTTCACATCAGAT | 567 |
| TGCACTGTCAAGAGCAGAT | 568 |
| TGCACTGTCAGCCTATGAT | 569 |
| TGCACTGTCATCACCTGAT | 570 |
| TGCACTTGTCATGTCTGAT | 571 |
| TGCACTGTTCATGCAGATGAT | 572 |
| TGCACTGTCAATGCATGCGAT | 573 |
| TGCACTGTGTAGGCATGAT | 574 |
| TGCACTGTGTCATCCTGAT | 575 |
| TGCACTTGTGTCATGAGAT | 576 |
| TGCACTGTTGTGATCAGAT | 577 |
| TGCACTGTGTTGCGATGAT | 578 |
| TGCACTGTGACAAGCTGAT | 579 |
| TGCACTGTGACATAATGAT | 580 |
| TGCACTTGTGAGTGCTGAT | 581 |
| TGCACTGTTGAGCTCAGAT | 582 |
| TGCACTGTGAATATGCGAT | 583 |
| TGCACTGTGATCCGCAGAT | 584 |
| TGCACTGTGATGTAATGAT | 585 |
| TGCACTTGTGATGACTGAT | 586 |
| TGCACTGTTGCGTGATGAT | 587 |
| TGCACTGTGCCGATCTGAT | 588 |
| TGCACTGTGCGCCATAGAT | 589 |
| TGCACTGTGCTCACCAGAT | 590 |
| TGCACTTGTGCTCAGTGAT | 591 |
| TGCACTGTTGCTGTGCGAT | 592 |
| TGCACTGTGCCTGAGAGAT | 593 |
| TGCACTGTGCAGGAGTGAT | 594 |
| TGCACTGTGCATCTTAGAT | 595 |
| TGCACTGTGCATCTGCCTGAT | 596 |
| TGCACTTGACTGCTGCATGAT | 597 |
| TGCACTGAACACATGCGAT | 598 |
| TGCACTGACAAGATCTGAT | 599 |
| TGCACTGAGTGAAGCTGAT | 600 |
| TGCACTGAGTGATGGAGAT | 601 |
| TGCACTTGAGACATGAGAT | 602 |
| TGCACTGAAGATCAGCATGAT | 603 |
| TGCACTGAGAATGTGTGAT | 604 |
| TGCACTGAGATGGATCGAT | 605 |
| TGCACTGAGCGCTGGCGAT | 606 |
| TGCACTTGAGCGCACTGAT | 607 |
| TGCACTGAAGCTATATGAT | 608 |
| TGCACTGAGCCTGTCAGAT | 609 |
| TGCACTGAGCAGGTGCATGAT | 610 |
| TGCACTGAGCAGCAAGATGAT | 611 |
| TGCACTTGAGCATAGAGAT | 612 |
| TGCACTGAAGCATATGCTGAT | 613 |
| TGCACTGAGCCATCATCAGAT | 614 |
| TGCACTGAGCATTGCGCTGAT | 615 |
| TGCACTGATACAGAATGAT | 616 |
| TGCACTTGATATCAGCGAT | 617 |
| TGCACTGAATATGCTGCTGAT | 618 |
| TGCACTGATAATGCACATGAT | 619 |
| TGCACTGATCGAATCAGAT | 620 |
| TGCACTGATCGCTCCTGAT | 621 |
| TGCACTGATCTATGCAATGAT | 622 |
| TGCACTTGATCTCGCTGAT | 623 |
| TGCACTGAATCTGTGAGAT | 624 |
| TGCACTGATCCTGCACGAT | 625 |
| TGCACTGATCACCTGAGAT | 626 |
| TGCACTGATCAGTGGCGAT | 627 |
| TGCACTTGATCATACAGAT | 628 |
| TGCACTGAATCATGCATAGAT | 629 |
| TGCACTGATGGTGCTCATGAT | 630 |
| TGCACTGATGAGGATCATGAT | 631 |
| TGCACTGATGAGCTTGATGAT | 632 |
| TGCACTGATGAGCAGCCAGAT | 633 |
| TGCACTTGATGATAGTGAT | 634 |
| TGCACTGAATGATCTCGAT | 635 |
| TGCACTGATGGCGCGCATGAT | 636 |
| TGCACTGATGCTTAGCGAT | 637 |
| TGCACTGATGCATCCGATGAT | 638 |
| TGCACTTGCGTGCATAGAT | 639 |
| TGCACTGCCGAGCAGCATGAT | 640 |
| TGCACTGCGAATATATGAT | 641 |
| TGCACTGCGATCCACAGAT | 642 |
| TGCACTGCGATGTGGCATGAT | 643 |
| TGCACTGCGCTATGCAATGAT | 644 |
| TGCACTTGCGCTCTCAGAT | 645 |
| TGCACTGCCGCTGCTGATGAT | 646 |
| TGCACTGCGCCTGCACATGAT | 647 |
| TGCACTGCGCAGGATAGAT | 648 |
| TGCACTGCGCAGCTTGCAGAT | 649 |
| TGCACTGCGCAGCATCCTGAT | 650 |
| TGCACTTGCGCATCAGCTGAT | 651 |
| TGCACTGCCGCATGAGCAGAT | 652 |
| TGCACTGCGCCATGATGTGAT | 653 |
| TGCACTGCTACAAGCAGAT | 654 |
| TGCACTGCTATCTGGTGAT | 655 |
| TGCACTTGCTATGAGCGAT | 656 |
| TGCACTGCCTATGCTAGAT | 657 |
| TGCACTGCTCCGCATCGAT | 658 |
| TGCACTGCTCTCCATGCTGAT | 659 |
| TGCACTGCTCTGCTTCATGAT | 660 |
| TGCACTTGCTCAGTGTGAT | 661 |
| TGCACTGCCTCAGATCATGAT | 662 |
| TGCACTGCTCCAGCGAGAT | 663 |
| TGCACTGCTCATTGATGAGAT | 664 |
| TGCACTGCTGTCTGGCATGAT | 665 |
| TGCACTTGCTGTGCGCGAT | 666 |
| TGCACTGCCTGATCAGATGAT | 667 |
| TGCACTGCTGGATGTCGAT | 668 |
| TGCACTGCTGCGGAGCATGAT | 669 |
| TGCACTGCTGCTGAACGAT | 670 |
| TGCACTGCTGCATCATTCGAT | 671 |
| TGCACTTGCACGATGAGAT | 672 |
| TGCACTGCCACGCGATGAT | 673 |
| TGCACTGCACCGCTCAGAT | 674 |
| TGCACTGCACTAAGCAGAT | 675 |
| TGCACTGCACTCACCTGAT | 676 |
| TGCACTGCACACTGCAATGAT | 677 |
| TGCACTTGCACAGAGCGAT | 678 |
| TGCACTGCCACATCGTGAT | 679 |
| TGCACTGCACCATCATATGAT | 680 |
| TGCACTGCACATTGTAGAT | 681 |
| TGCACTGCAGTGATTCATGAT | 682 |
| TGCACTGCAGTGCTGCCTGAT | 683 |
| TGCACTTGCAGTGCAGATGAT | 684 |
| TGCACTGCCAGACTGTGAT | 685 |
| TGCACTGCAGGACATCATGAT | 686 |
| TGCACTGCAGAGGATGCTGAT | 687 |
| TGCACTGCAGATGCCTATGAT | 688 |
| TGCACTTGCAGCGAGTGAT | 689 |
| TGCACTGCCAGCACAGCAGAT | 690 |
| TGCACTGCAGGCATCTCTGAT | 691 |
| TGCACTGCAGCAATGCACGAT | 692 |
| TGCACTGCATAGATTAGAT | 693 |
| TGCACTTGCATAGCGTGAT | 694 |
| TGCACTGCCATAGCACGAT | 695 |
| TGCACTGCATTATATCATGAT | 696 |
| TGCACTGCATATTGTGATGAT | 697 |
| TGCACTGCATCGTGGCATGAT | 698 |
| TGCACTGCATCTCTGAATGAT | 699 |
| TGCACTTGCATCTGACATGAT | 700 |
| TGCACTGCCATCATAGATGAT | 701 |
| TGCACTGCATTCATCTGCGAT | 702 |
| TGCACTGCATGTTGCTGAGAT | 703 |
| TGCACTGCATGACAATGCGAT | 704 |
| TGCACTGCATGATAGCCAGAT | 705 |
| TGCACTTGCATGCGATGCGAT | 706 |
| TGCACTGCCATGCTATGAGAT | 707 |
| TGCACTGCATTGCTCGCAGAT | 708 |
| TGCACTGCATGCCTGTCTGAT | 709 |
| TGCACTGCATGCTGGCGTGAT | 710 |
| TGCACTGCATGCACACCTGAT | 711 |
| TGCACTTGCATGCAGTCAGAT | 712 |
| TGCACTGCCATGCAGCGCGAT | 713 |
| TGCACACGTGGCACATGAT | 714 |
| TGCACACGTGCAATGCGAT | 715 |
| TGCACACGAGCGCAATGAT | 716 |
| TGCACAACGAGCATATGAT | 717 |
| TGCACACGGATAGCATGAT | 718 |
| TGCACACGATTCTGATGAT | 719 |
| TGCACACGCGAGGCATGAT | 720 |
| TGCACACGCGCATGGAGAT | 721 |
| TGCACAACGCTATGCTGAT | 722 |
| TGCACACGGCTCAGATGAT | 723 |
| TGCACACGCTTGATCAGAT | 724 |
| TGCACACGCTGCCGCAGAT | 725 |
| TGCACACGCACTGCCAGAT | 726 |
| TGCACACGCAGCATGCCTGAT | 727 |
| TGCACAACGCATATGAGAT | 728 |
| TGCACACGGCATCACTGAT | 729 |
| TGCACACGCAATGTGCATGAT | 730 |
| TGCACACGCATGGAGCGAT | 731 |
| TGCACACTAGCATGGCGAT | 732 |
| TGCACAACTATCTGCAGAT | 733 |
| TGCACACTTATCATGTGAT | 734 |
| TGCACACTATTGATGCATGAT | 735 |
| TGCACACTCGCAATATGAT | 736 |
| TGCACACTCTCTCAATGAT | 737 |
| TGCACAACTCTCATGAGAT | 738 |
| TGCACACTTCTGCGATGAT | 739 |
| TGCACACTCTTGCATCGAT | 740 |
| TGCACACTCACAATGCATGAT | 741 |
| TGCACACTCAGCGCCAGAT | 742 |
| TGCACAACTCATATGCGAT | 743 |
| TGCACACTTCATGTATGAT | 744 |
| TGCACACTCAATGCTGCTGAT | 745 |
| TGCACACTCATGGCACATGAT | 746 |
| TGCACACTGTCAGCCAGAT | 747 |
| TGCACAACTGTCATCTGAT | 748 |
| TGCACACTTGTGCTATGAT | 749 |
| TGCACACTGAATGTGCGAT | 750 |
| TGCACACTGATGGCGTGAT | 751 |
| TGCACACTGATGCAACGAT | 752 |
| TGCACACTGATGCATGGAGAT | 753 |
| TGCACAACTGCGCTCTGAT | 754 |
| TGCACACTTGCTCTGTGAT | 755 |
| TGCACACTGCCTGATGATGAT | 756 |
| TGCACACTGCTGGCAGCTGAT | 757 |
| TGCACACTGCACGAATGAT | 758 |
| TGCACAACTGCACATAGAT | 759 |
| TGCACACTTGCAGATCGAT | 760 |
| TGCACACTGCCATATCATGAT | 761 |
| TGCACACTGCATTCTCGAT | 762 |
| TGCACACACGCATGGCATGAT | 763 |
| TGCACAACACTCTGCAGAT | 764 |
| TGCACACAACTCATATGAT | 765 |
| TGCACACACTTGATGCGAT | 766 |
| TGCACACACACAACATGAT | 767 |
| TGCACACACAGATAATGAT | 768 |
| TGCACAACACAGCTGTGAT | 769 |
| TGCACACAACATATCAGAT | 770 |
| TGCACACACAATATGTGAT | 771 |
| TGCACACACATCCAGCGAT | 772 |
| TGCACACACATGAGGCATGAT | 773 |
| TGCACAACACATGCTAGAT | 774 |
| TGCACACAACATGCATCTGAT | 775 |
| TGCACACAGTTATGCAGAT | 776 |
| TGCACACAGTCAATGTGAT | 777 |
| TGCACACAGTGCGAATGAT | 778 |
| TGCACAACAGTGCATAGAT | 779 |
| TGCACACAAGACATGAGAT | 780 |
| TGCACACAGAAGATGCGAT | 781 |
| TGCACACAGATAATCTGAT | 782 |
| TGCACACAGATCGCCAGAT | 783 |
| TGCACAACAGATGTATGAT | 784 |
| TGCACACAAGATGACAGAT | 785 |
| TGCACACAGAATGCTCGAT | 786 |
| TGCACACAGATGGCAGCTGAT | 787 |
| TGCACACAGCGCTCCAGAT | 788 |
| TGCACAACAGCTCTCTGAT | 789 |
| TGCACACAAGCTCACAGAT | 790 |
| TGCACACAGCCTGTGTGAT | 791 |
| TGCACACAGCACCGCTGAT | 792 |
| TGCACACAGCACTAATGAT | 793 |
| TGCACACAGCAGCAGAATGAT | 794 |
| TGCACAACATACATCAGAT | 795 |
| TGCACACAATAGCGATGAT | 796 |
| TGCACACATAAGCACTGAT | 797 |
| TGCACACATATAAGATGAT | 798 |
| TGCACACATATCTCCTGAT | 799 |
| TGCACAACATATGTCAGAT | 800 |
| TGCACACAATATGTGTGAT | 801 |
| TGCACACATAATGAGCGAT | 802 |
| TGCACACATATGGCATATGAT | 803 |
| TGCACACATCTATGGCATGAT | 804 |
| TGCACAACATCTGATAGAT | 805 |
| TGCACACAATCTGCAGCAGAT | 806 |
| TGCACACATCCTGCATGTGAT | 807 |
| TGCACACATCACCAGTGAT | 808 |
| TGCACACATCAGTGGCGAT | 809 |
| TGCACACATCAGCTCAATGAT | 810 |
| TGCACAACATCAGCATGAGAT | 811 |
| TGCACACAATCATCGCATGAT | 812 |
| TGCACACATGGTACATGAT | 813 |
| TGCACACATGTCCTGAGAT | 814 |
| TGCACACATGTGAGGAGAT | 815 |
| TGCACAACATGTGATCGAT | 816 |
| TGCACACAATGTGCGCGAT | 817 |
| TGCACACATGGACTGTGAT | 818 |
| TGCACACATGACCAGCGAT | 819 |
| TGCACACATGAGTGGCATGAT | 820 |
| TGCACAACATGAGATAGAT | 821 |
| TGCACACAATGCGAGCGAT | 822 |
| TGCACACATGGCGATCATGAT | 823 |
| TGCACACATGCGGCGCATGAT | 824 |
| TGCACACATGCTCAATGCGAT | 825 |
| TGCACACATGCTGTGCCAGAT | 826 |
| TGCACAACATGCACATCTGAT | 827 |
| TGCACACAATGCAGATGTGAT | 828 |
| TGCACACATGGCAGCACAGAT | 829 |
| TGCACACATGCAATAGCTGAT | 830 |
| TGCACACATGCATCCAGAGAT | 831 |
| TGCACACATGCATGTCCTGAT | 832 |
| TGCACAAGTAGCATCAGAT | 833 |
| TGCACAGTTATGTGCTGAT | 834 |
| TGCACAGTATTGCTGAGAT | 835 |
| TGCACAGTCGCTTGATGAT | 836 |
| TGCACAGTCTGATCCTGAT | 837 |
| TGCACAAGTCTGCTCAGAT | 838 |
| TGCACAGTTCTGCAGTGAT | 839 |
| TGCACAGTCAACAGCAGAT | 840 |
| TGCACAGTCAGTTGCAGAT | 841 |
| TGCACAGTCAGATAATGAT | 842 |
| TGCACAAGTCAGCACTGAT | 843 |
| TGCACAGTTCATCTGTGAT | 844 |
| TGCACAGTCAATGAGCGAT | 845 |
| TGCACAGTGTATTGCTGAT | 846 |
| TGCACAGTGTCAGAATGAT | 847 |
| TGCACAAGTGTGCGCAGAT | 848 |
| TGCACAGTTGTGCTGTGAT | 849 |
| TGCACAGTGAACGCATGAT | 850 |
| TGCACAGTGACAATGTGAT | 851 |
| TGCACAGTGAGCTAATGAT | 852 |
| TGCACAAGTGAGCTGCGAT | 853 |
| TGCACAGTTGATACATGAT | 854 |
| TGCACAGTGAATCATCGAT | 855 |
| TGCACAGTGATGGTCAGAT | 856 |
| TGCACAGTGCGACAATGAT | 857 |
| TGCACAAGTGCGATGAGAT | 858 |
| TGCACAGTTGCGCATGCTGAT | 859 |
| TGCACAGTGCCTAGCAGAT | 860 |
| TGCACAGTGCTCCATAGAT | 861 |
| TGCACAGTGCTGTGGCATGAT | 862 |
| TGCACAAGTGCAGCGAGAT | 863 |
| TGCACAGTTGCATCTGCAGAT | 864 |
| TGCACAGACGGATGCAGAT | 865 |
| TGCACAGACGCAATCTGAT | 866 |
| TGCACAGACTCACAATGAT | 867 |
| TGCACAAGACTGATATGAT | 868 |
| TGCACAGAACTGCGATGAT | 869 |
| TGCACAGACTTGCTGCGAT | 870 |
| TGCACAGACACAATATGAT | 871 |
| TGCACAGACATCTGGCATGAT | 872 |
| TGCACAAGACATCAGAGAT | 873 |
| TGCACAGAACATGTCAGAT | 874 |
| TGCACAGAGTTCATATGAT | 875 |
| TGCACAGAGTGCCGCTGAT | 876 |
| TGCACAGAGACACAATGAT | 877 |
| TGCACAAGAGAGAGCTGAT | 878 |
| TGCACAGAAGAGATATGAT | 879 |
| TGCACAGAGAAGCGATGAT | 880 |
| TGCACAGAGAGCCATGCAGAT | 881 |
| TGCACAGAGATCTGGTGAT | 882 |
| TGCACAGAGATGTGCAATGAT | 883 |
| TGCACAAGAGATGCATCTGAT | 884 |
| TGCACAGAAGCGTGCTGAT | 885 |
| TGCACAGAGCCGAGATGAT | 886 |
| TGCACAGAGCGCCGCAGAT | 887 |
| TGCACAGAGCTAGCCTGAT | 888 |
| TGCACAAGAGCTGACAGAT | 889 |
| TGCACAGAAGCTGCATGAGAT | 890 |
| TGCACAGAGCCACTCAGAT | 891 |
| TGCACAGAGCACCAGCGAT | 892 |
| TGCACAGAGCATGAATGTGAT | 893 |
| TGCACAGAGCATGCTAATGAT | 894 |
| TGCACAAGATACTCATGAT | 895 |
| TGCACAGAATACATGCGAT | 896 |
| TGCACAGATAAGAGCAGAT | 897 |
| TGCACAGATAGCCGCTGAT | 898 |
| TGCACAGATATAGCCTGAT | 899 |
| TGCACAAGATATATATGAT | 900 |
| TGCACAGAATATGCAGATGAT | 901 |
| TGCACAGATCCGATGTGAT | 902 |
| TGCACAGATCGCCACAGAT | 903 |
| TGCACAGATCTATCCAGAT | 904 |
| TGCACAGATCTCATGAATGAT | 905 |
| TGCACAAGATCTGAGAGAT | 906 |
| TGCACAGAATCAGTCTGAT | 907 |
| TGCACAGATCCATCATCTGAT | 908 |
| TGCACAGATCATTGTGATGAT | 909 |
| TGCACAGATCATGCCGCAGAT | 910 |
| TGCACAAGATGTATGAGAT | 911 |
| TGCACAGAATGTCTGCATGAT | 912 |
| TGCACAGATGGTCACAGAT | 913 |
| TGCACAGATGTGGATCATGAT | 914 |
| TGCACAGATGACATTAGAT | 915 |
| TGCACAGATGATGATGGCGAT | 916 |
| TGCACAAGATGCTCGTGAT | 917 |
| TGCACAGAATGCTGTCGAT | 918 |
| TGCACAGATGGCTGCAGCGAT | 919 |
| TGCACAGATGCAACAGATGAT | 920 |
| TGCACAGATGCATGGATAGAT | 921 |
| TGCACAGCGTCATGCAATGAT | 922 |
| TGCACAAGCGACATGCGAT | 923 |
| TGCACAGCCGATATCAGAT | 924 |
| TGCACAGCGAATGATGATGAT | 925 |
| TGCACAGCGATGGCGCGAT | 926 |
| TGCACAGCGCGCTGGCATGAT | 927 |
| TGCACAAGCGCTCTGAGAT | 928 |
| TGCACAGCCGCTGCTCGAT | 929 |
| TGCACAGCGCCTGCATGTGAT | 930 |
| TGCACAGCGCACCAGCATGAT | 931 |
| TGCACAGCGCATAGGTGAT | 932 |
| TGCACAGCGCATGATCCTGAT | 933 |
| TGCACAAGCGCATGCGATGAT | 934 |
| TGCACAGCCGCATGCACAGAT | 935 |
| TGCACAGCTAAGCAGCATGAT | 936 |
| TGCACAGCTCGAATGCGAT | 937 |
| TGCACAGCTCTCAGGCATGAT | 938 |
| TGCACAAGCTCACTGAGAT | 939 |
| TGCACAGCCTCAGCGTGAT | 940 |
| TGCACAGCTCCATCATCAGAT | 941 |
| TGCACAGCTCATTGCAGAGAT | 942 |
| TGCACAGCTGTGACCAGAT | 943 |
| TGCACAAGCTGACTCAGAT | 944 |
| TGCACAGCCTGATCTGCTGAT | 945 |
| TGCACAGCTGGATGAGCTGAT | 946 |
| TGCACAGCTGCGGCTAGAT | 947 |
| TGCACAGCTGCTACCTGAT | 948 |
| TGCACAGCTGCAGTGAATGAT | 949 |
| TGCACAAGCTGCAGAGCAGAT | 950 |
| TGCACAGCCTGCATCTATGAT | 951 |
| TGCACAGCACCTGCATCAGAT | 952 |
| TGCACAGCACACCATGCAGAT | 953 |
| TGCACAGCACAGAGGAGAT | 954 |
| TGCACAGCACATGCGCCTGAT | 955 |
| TGCACAAGCAGTGAGCGAT | 956 |
| TGCACAGCCAGTGCTCATGAT | 957 |
| TGCACAGCAGGAGCTAGAT | 958 |
| TGCACAGCAGATTCACGAT | 959 |
| TGCACAGCAGATCAAGATGAT | 960 |
| TGCACAAGCAGCGATCGAT | 961 |
| TGCACAGCCAGCGCAGCTGAT | 962 |
| TGCACAGCAGGCTATAGAT | 963 |
| TGCACAGCAGCTTCGCGAT | 964 |
| TGCACAGCAGCAGTTGCAGAT | 965 |
| TGCACAGCAGCATAGCCAGAT | 966 |
| TGCACAAGCATAGTATGAT | 967 |
| TGCACAGCCATAGATCGAT | 968 |
| TGCACAGCATTAGCATGTGAT | 969 |
| TGCACAGCATATTACAGAT | 970 |
| TGCACAGCATATCGGTGAT | 971 |
| TGCACAAGCATATCTAGAT | 972 |
| TGCACAGCCATATGATGAGAT | 973 |
| TGCACAGCATTATGCTGCGAT | 974 |
| TGCACAGCATCGGCTCGAT | 975 |
| TGCACAGCATCGCAAGATGAT | 976 |
| TGCACAGCATCTCTGCCTGAT | 977 |
| TGCACAAGCATCTGACGAT | 978 |
| TGCACAGCCATCTGCTGAGAT | 979 |
| TGCACAGCATTCAGACATGAT | 980 |
| TGCACAGCATCAAGCAGCGAT | 981 |
| TGCACAGCATGTGAATGTGAT | 982 |
| TGCACAGCATGAGCGCCAGAT | 983 |
| TGCACAAGCATGCTCTCAGAT | 984 |
| TGCACAGCCATGCACTGCGAT | 985 |
| TGCACATACGGCATGCGAT | 986 |
| TGCACATACTGCCTATGAT | 987 |
| TGCACATACTGCAGGAGAT | 988 |
| TGCACAATACACATCTGAT | 989 |
| TGCACATAACAGTGCAGAT | 990 |
| TGCACATACAAGAGCTGAT | 991 |
| TGCACATACAGCCGATGAT | 992 |
| TGCACATACATAGAATGAT | 993 |
| TGCACAATACATGATCGAT | 994 |
| TGCACATAACATGCTGCTGAT | 995 |
| TGCACATAGTTCATCTGAT | 996 |
| TGCACATAGTGAATATGAT | 997 |
| TGCACATAGTGATGGCGAT | 998 |
| TGCACATAGTGCTGCAATGAT | 999 |
| TGCACAATAGACAGCTGAT | 1000 |
| TGCACATAAGACATATGAT | 1001 |
| TGCACATAGAAGATGTGAT | 1002 |
| TGCACATAGAGCCTCAGAT | 1003 |
| TGCACATAGATAGCCAGAT | 1004 |
| TGCACAATAGATGTGAGAT | 1005 |
| TGCACATAAGATGCGTGAT | 1006 |
| TGCACATAGAATGCACGAT | 1007 |
| TGCACATAGCGTTCATGAT | 1008 |
| TGCACATAGCGAGCCAGAT | 1009 |
| TGCACAATAGCTATGAGAT | 1010 |
| TGCACATAAGCTCAGTGAT | 1011 |
| TGCACATAGCCTGACTGAT | 1012 |
| TGCACATAGCTGGCATCAGAT | 1013 |
| TGCACATAGCAGCAACATGAT | 1014 |
| TGCACAATAGCATCGAGAT | 1015 |
| TGCACATAAGCATCTCATGAT | 1016 |
| TGCACATATAACATGCATGAT | 1017 |
| TGCACATATAGCCTATGAT | 1018 |
| TGCACATATAGCAGGAGAT | 1019 |
| TGCACAATATATATGTGAT | 1020 |
| TGCACATAATATCTGCGAT | 1021 |
| TGCACATATAATCACAGAT | 1022 |
| TGCACATATATGGTGCATGAT | 1023 |
| TGCACATATATGACCTGAT | 1024 |
| TGCACAATATCGATATGAT | 1025 |
| TGCACATAATCGCGCTGAT | 1026 |
| TGCACATATCCTCGCAGAT | 1027 |
| TGCACATATCTCCTGTGAT | 1028 |
| TGCACATATCTGTCCAGAT | 1029 |
| TGCACAATATCTGAGTGAT | 1030 |
| TGCACATAATCTGCACATGAT | 1031 |
| TGCACATATCCACAGCGAT | 1032 |
| TGCACATATCATTATCATGAT | 1033 |
| TGCACATATCATCTTAGAT | 1034 |
| TGCACATATCATGAGCCAGAT | 1035 |
| TGCACAATATGTCGATGAT | 1036 |
| TGCACATAATGTCAGCGAT | 1037 |
| TGCACATATGGTGACAGAT | 1038 |
| TGCACATATGACCTGAGAT | 1039 |
| TGCACATATGAGATTCGAT | 1040 |
| TGCACATATGATGAGAATGAT | 1041 |
| TGCACAATATGATGCATAGAT | 1042 |
| TGCACATAATGCGTGAGAT | 1043 |
| TGCACATATGGCGCACGAT | 1044 |
| TGCACATATGCGGCAGATGAT | 1045 |
| TGCACATATGCTGTTGCTGAT | 1046 |
| TGCACAATATGCACGTGAT | 1047 |
| TGCACATAATGCAGCTGCGAT | 1048 |
| TGCACATATGGCATATGCGAT | 1049 |
| TGCACATCGAGCCATGCAGAT | 1050 |
| TGCACATCGATCATTCATGAT | 1051 |
| TGCACATCGATGCAGAATGAT | 1052 |
| TGCACAATCGCTCTATGAT | 1053 |
| TGCACATCCGCTCATCGAT | 1054 |
| TGCACATCGCCTGCTGCTGAT | 1055 |
| TGCACATCGCACCAGAGAT | 1056 |
| TGCACATCGCAGAGGTGAT | 1057 |
| TGCACATCGCAGCTGAATGAT | 1058 |
| TGCACAATCGCATCGTGAT | 1059 |
| TGCACATCCGCATGCATAGAT | 1060 |
| TGCACATCTAACACATGAT | 1061 |
| TGCACATCTAGCCATAGAT | 1062 |
| TGCACATCTATCAGGCGAT | 1063 |
| TGCACAATCTATGATCGAT | 1064 |
| TGCACATCCTATGCTCATGAT | 1065 |
| TGCACATCTCCTGATCATGAT | 1066 |
| TGCACATCTCTGGCTGCAGAT | 1067 |
| TGCACATCTCACTGGTGAT | 1068 |
| TGCACATCTCAGTGCAATGAT | 1069 |
| TGCACAATCTCAGCAGATGAT | 1070 |
| TGCACATCCTCAGCATCTGAT | 1071 |
| TGCACATCTCCATAGAGAT | 1072 |
| TGCACATCTCATTGATGTGAT | 1073 |
| TGCACATCTGTCATTAGAT | 1074 |
| TGCACAATCTGTGAGCGAT | 1075 |
| TGCACATCCTGTGCGCATGAT | 1076 |
| TGCACATCTGGTGCATGTGAT | 1077 |
| TGCACATCTGAGGATCATGAT | 1078 |
| TGCACATCTGAGCGGAGAT | 1079 |
| TGCACATCTGAGCTGCCTGAT | 1080 |
| TGCACAATCTGATATGATGAT | 1081 |
| TGCACATCCTGCGATAGAT | 1082 |
| TGCACATCTGGCGATGCTGAT | 1083 |
| TGCACATCTGCGGCACATGAT | 1084 |
| TGCACATCTGCTGTTCGAT | 1085 |
| TGCACATCTGCACATGGCGAT | 1086 |
| TGCACAATCTGCATACGAT | 1087 |
| TGCACATCCTGCATCGCAGAT | 1088 |
| TGCACATCACCTCAGCATGAT | 1089 |
| TGCACATCACTGGTGCATGAT | 1090 |
| TGCACATCACTGCAACGAT | 1091 |
| TGCACATCACACATGAATGAT | 1092 |
| TGCACAATCACAGCAGCAGAT | 1093 |
| TGCACATCCACATGCAGTGAT | 1094 |
| TGCACATCAGGTAGCTGAT | 1095 |
| TGCACATCAGTCCTGCGAT | 1096 |
| TGCACATCAGATATTAGAT | 1097 |
| TGCACAATCAGCGCGAGAT | 1098 |
| TGCACATCCAGCGCATGTGAT | 1099 |
| TGCACATCAGGCTATCATGAT | 1100 |
| TGCACATCAGCTTGTAGAT | 1101 |
| TGCACATCAGCTGAAGATGAT | 1102 |
| TGCACATCAGCACATCCAGAT | 1103 |
| TGCACAATCAGCAGACGAT | 1104 |
| TGCACATCCATAGATGATGAT | 1105 |
| TGCACATCATTAGCGCGAT | 1106 |
| TGCACATCATCGGAGCATGAT | 1107 |
| TGCACATCATCGATTCGAT | 1108 |
| TGCACAATCATCGCTAGAT | 1109 |
| TGCACATCCATCTCATCTGAT | 1110 |
| TGCACATCATTCACTGCAGAT | 1111 |
| TGCACATCATCAATGTGAGAT | 1112 |
| TGCACATCATCATGGCTCGAT | 1113 |
| TGCACATCATGTCTCAATGAT | 1114 |
| TGCACAATCATGTGCACTGAT | 1115 |
| TGCACATCCATGACGCATGAT | 1116 |
| TGCACATCATTGATCATCGAT | 1117 |
| TGCACATCATGCCTATGTGAT | 1118 |
| TGCACATCATGCTCCGCTGAT | 1119 |
| TGCACAATGTACTGATGAT | 1120 |
| TGCACATGGTAGAGATGAT | 1121 |
| TGCACATGTAATATCAGAT | 1122 |
| TGCACATGTATCCTCTGAT | 1123 |
| TGCACATGTATCAGGTGAT | 1124 |
| TGCACATGTATGCGCAATGAT | 1125 |
| TGCACAATGTATGCATATGAT | 1126 |
| TGCACATGGTCGATGCATGAT | 1127 |
| TGCACATGTCCGCAGAGAT | 1128 |
| TGCACATGTCTAAGATGAT | 1129 |
| TGCACATGTCTCTAATGAT | 1130 |
| TGCACAATGTCTCTGCGAT | 1131 |
| TGCACATGGTCTGACAGAT | 1132 |
| TGCACATGTCCACATGCTGAT | 1133 |
| TGCACATGTCATTCATGAGAT | 1134 |
| TGCACATGTGTGATTGATGAT | 1135 |
| TGCACAATGTGTGCTAGAT | 1136 |
| TGCACATGGTGTGCACGAT | 1137 |
| TGCACATGTGGACACAGAT | 1138 |
| TGCACATGTGAGGATGCAGAT | 1139 |
| TGCACATGTGAGCGGTGAT | 1140 |
| TGCACATGTGATGCAGGAGAT | 1141 |
| TGCACAATGTGCGAGCGAT | 1142 |
| TGCACATGGTGCGCTCATGAT | 1143 |
| TGCACATGTGGCTATCATGAT | 1144 |
| TGCACATGTGCTTCGCATGAT | 1145 |
| TGCACATGTGCAGCCATCGAT | 1146 |
| TGCACATGTGCATATGGTGAT | 1147 |
| TGCACAATGTGCATCAGCGAT | 1148 |
| TGCACATGGTGCATGTGAGAT | 1149 |
| TGCACATGACCGCTGTGAT | 1150 |
| TGCACATGACGCCAGCATGAT | 1151 |
| TGCACATGACGCATTAGAT | 1152 |
| TGCACAATGACTATCTGAT | 1153 |
| TGCACATGGACTCAGCGAT | 1154 |
| TGCACATGACCACGCAGAT | 1155 |
| TGCACATGACACCAGTGAT | 1156 |
| TGCACATGACAGTAATGAT | 1157 |
| TGCACAATGACAGCTCGAT | 1158 |
| TGCACATGGACATATGCAGAT | 1159 |
| TGCACATGACCATGACATGAT | 1160 |
| TGCACATGAGTAATGCATGAT | 1161 |
| TGCACATGAGTGCTTCGAT | 1162 |
| TGCACATGAGTGCAGCCAGAT | 1163 |
| TGCACAATGAGACTGCGAT | 1164 |
| TGCACATGGAGATACTGAT | 1165 |
| TGCACATGAGGCTCTGATGAT | 1166 |
| TGCACATGAGCAAGATGAGAT | 1167 |
| TGCACATGAGCATGGTCTGAT | 1168 |
| TGCACATGAGCATGAGGCGAT | 1169 |
| TGCACAATGATAGTGTGAT | 1170 |
| TGCACATGGATAGCTGCAGAT | 1171 |
| TGCACATGATTATGTCGAT | 1172 |
| TGCACATGATCGGACTGAT | 1173 |
| TGCACATGATCTGAATGCGAT | 1174 |
| TGCACATGATCACACAATGAT | 1175 |
| TGCACAATGATGTCATGTGAT | 1176 |
| TGCACATGGATGACATCTGAT | 1177 |
| TGCACATGATTGATCGCTGAT | 1178 |
| TGCACATGATGAATCTATGAT | 1179 |
| TGCACATGATGCTCCTCTGAT | 1180 |
| TGCACATGATGCTCAGGAGAT | 1181 |
| TGCACAATGATGCTGTATGAT | 1182 |
| TGCACATGGATGCAGACAGAT | 1183 |
| TGCACATGCGGTATGCGAT | 1184 |
| TGCACATGCGTCCTGTGAT | 1185 |
| TGCACATGCGTCACCTGAT | 1186 |
| TGCACATGCGTGAGCAATGAT | 1187 |
| TGCACAATGCGTGCTGCAGAT | 1188 |
| TGCACATGGCGACTGCATGAT | 1189 |
| TGCACATGCGGAGTGAGAT | 1190 |
| TGCACATGCGAGGAGCGAT | 1191 |
| TGCACATGCGAGCTTCGAT | 1192 |
| TGCACATGCGAGCATGGTGAT | 1193 |
| TGCACAATGCGATCATGAGAT | 1194 |
| TGCACATGGCGCGATCATGAT | 1195 |
| TGCACATGCGGCGCAGCAGAT | 1196 |
| TGCACATGCGCTTACAGAT | 1197 |
| TGCACATGCGCTGAATGAGAT | 1198 |
| TGCACAATGCGCACACGAT | 1199 |
| TGCACATGGCGCAGTGCTGAT | 1200 |
| TGCACATGCGGCATCTGCGAT | 1201 |
| TGCACATGCGCAATGTATGAT | 1202 |
| TGCACATGCTAGTGGCGAT | 1203 |
| TGCACATGCTATAGCAATGAT | 1204 |
| TGCACAATGCTATCGAGAT | 1205 |
| TGCACATGGCTATGCACTGAT | 1206 |
| TGCACATGCTTCGTATGAT | 1207 |
| TGCACATGCTCGGCTGCTGAT | 1208 |
| TGCACATGCTCTATTGCAGAT | 1209 |
| TGCACATGCTCTGAGCCTGAT | 1210 |
| TGCACAATGCTCTGCATAGAT | 1211 |
| TGCACATGGCTCACATATGAT | 1212 |
| TGCACATGCTTCAGCTCAGAT | 1213 |
| TGCACATGCTCAATATCTGAT | 1214 |
| TGCACATGCTCATGGCGCGAT | 1215 |
| TGCACAATGCTGTAGTGAT | 1216 |
| TGCACATGGCTGTCTCGAT | 1217 |
| TGCACATGCTTGTGTCATGAT | 1218 |
| TGCACATGCTGAATGTGTGAT | 1219 |
| TGCACATGCTGCGTTGCAGAT | 1220 |
| TGCACATGCTGCGCGAATGAT | 1221 |
| TGCACAATGCTGCACGCTGAT | 1222 |
| TGCACATGGCTGCAGACTGAT | 1223 |
| TGCACATGCTTGCATATAGAT | 1224 |
| TGCACATGCACGGTGCGAT | 1225 |
| TGCACATGCACTAGGTGAT | 1226 |
| TGCACAATGCACTCGAGAT | 1227 |
| TGCACATGGCACTCTCATGAT | 1228 |
| TGCACATGCAACACTAGAT | 1229 |
| TGCACATGCACAAGATCAGAT | 1230 |
| TGCACATGCACAGAATGTGAT | 1231 |
| TGCACATGCACAGCACCTGAT | 1232 |
| TGCACAATGCACATACGAT | 1233 |
| TGCACATGGCAGTCGCATGAT | 1234 |
| TGCACATGCAAGTGTGATGAT | 1235 |
| TGCACATGCAGAAGTCATGAT | 1236 |
| TGCACATGCAGAGAAGATGAT | 1237 |
| TGCACATGCAGAGCGCCTGAT | 1238 |
| TGCACAATGCAGAGCACAGAT | 1239 |
| TGCACATGGCAGATATGTGAT | 1240 |
| TGCACATGCAAGATCTCAGAT | 1241 |
| TGCACATGCAGAATGTGCGAT | 1242 |
| TGCACATGCAGATGGCGAGAT | 1243 |
| TGCACATGCAGCGCTAATGAT | 1244 |
| TGCACAATGCAGCACGATGAT | 1245 |
| TGCACATGGCATACTGCTGAT | 1246 |
| TGCACATGCAATACATGAGAT | 1247 |
| TGCACATGCATAAGAGCTGAT | 1248 |
| TGCACATGCATATAATGCGAT | 1249 |
| TGCACATGCATCGCGCCAGAT | 1250 |
| TGCACAATGCATCTATATGAT | 1251 |
| TGCACATGGCATCTGTGTGAT | 1252 |
| TGCACATGCAATCACATCGAT | 1253 |
| TGCACATGCATGGTACGAT | 1254 |
| TGCACATGCATGTGGATAGAT | 1255 |
| TGCACATGCATGCGAGGAGAT | 1256 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A composition, comprising a non-naturally occurring nucleic acid barcode molecule comprising a sequence of any one of SEQ ID NOs: 1-1256.

2. The composition of claim 1, wherein said non-naturally occurring nucleic acid barcode molecule is coupled to a support.

3. The composition of claim 2, wherein said support is a bead.

4. (canceled)

5. (canceled)

6. The composition of claim 1, wherein said non-naturally occurring nucleic acid barcode molecule comprises a sequence of any one of SEQ ID NOs: 1-238.

7. The composition of claim 1, wherein said non-naturally occurring nucleic acid barcode molecule comprises a sequence of any one of SEQ ID NOs: 239-1256.

8. The composition of claim 1, wherein said composition comprises a plurality of non-naturally occurring nucleic acid barcode molecules comprising at least 96 different sequences selected from the group consisting of SEQ ID NOs: 1-238.

9. The composition of claim 1, wherein said composition comprises a plurality of non-naturally occurring nucleic acid barcode molecules comprising at least 96 different sequences selected from the group consisting of SEQ ID NOs: 239-1256.

10. A computer-implemented method for generating or selecting a set of barcode sequences, comprising:
(a) providing, by at least one processor, a plurality of barcode sequences;
(b) generating, by said at least one processor, a plurality of matrices of flow data, wherein each matrix of said plurality of matrices of flow data corresponds to a different barcode sequence of said plurality of barcode sequences, and wherein a given matrix of said plurality of matrices of flow data comprises information on a plurality of flow cycles that is representative of nucleotide incorporation events corresponding to a given barcode sequence of said plurality of barcode sequences;
(c) applying, by said at least one processor, one or more constraints on said plurality of matrices of flow data, thereby generating a first set of filtered matrices;
(d) filtering, by said at least one processor, said first set of filtered matrices using one or more criteria to generate a third set of filtered matrices corresponding to said set of barcode sequences, wherein said set of barcode sequences is a subset of barcode sequences of said plurality of barcode sequences; and
(e) electronically outputting said set of barcode sequences.

11. The computer-implemented method of claim 10, wherein each barcode sequence of said set of barcode sequences is from 9 to 30 nucleotides in length.

12. The computer-implemented method of claim 10, wherein each barcode sequence of said set of barcode sequences is from 9 to 11 nucleotides in length.

13. The computer-implemented method of claim 10, wherein said plurality of matrices of flow data comprises a 1×N vector, wherein N is a number of flow cycles in said plurality of flow cycles.

14. The computer-implemented method of claim 10, wherein said one or more criteria comprises barcode sequence length, and wherein said filtering in (c) comprises removing matrices corresponding to barcode sequences that have a sequence length that is greater or less than a predetermined threshold value, thereby yielding a second set of filtered matrices.

15. The computer-implemented method of claim 14, wherein a given matrix of said plurality of matrices of flow data, said first set of filtered matrices, or said second set of filtered matrices comprises a 1×N vector, wherein N is a number of flow cycles in said plurality of flow cycles, wherein each element of said 1×N vector is an H-mer representative of said nucleotide incorporation events, and wherein H corresponds to a number of nucleotides incorporated per flow cycle of said plurality of flow cycles.

16. The computer-implemented method of claim 15, wherein (c) further comprises calculating, using said at least one processor, an edit distance between said given matrix and another matrix of said plurality of matrices of flow data, said first set of filtered matrices, or said second set of filtered matrices, and wherein said one or more criteria in (d) comprise a predetermined threshold or a range of edit distances.

17. The computer-implemented method of claim 16, wherein said edit distance is calculated by counting, using said at least one processor, a number of different elements between two matrices of said second set of filtered matrices.

18. The computer-implemented method of claim 16, wherein said predetermined threshold or said range of edit distances is at least 2.

19. (canceled)

20. The computer-implemented method of claim 15, wherein said one or more constraints in (b) comprises a minimum, a maximum, or a range of one or more parameters selected from the group consisting of: said number of flow cycles, H-mer magnitude, and a number of H-mers above a predetermined threshold H value.

21. The computer-implemented method of claim 20, wherein said predetermined threshold H value is 7.

22. The computer-implemented method of claim 10, wherein said electronically outputting in (e) comprises presenting, on a user interface, said set of barcode sequences.

23. A kit, comprising: at least 96 non-naturally occurring nucleic acid barcode molecules, wherein each of said at least 96 non-naturally occurring nucleic acid barcode molecules comprises a different sequence selected from the group consisting of SEQ ID NOs: 239-1256.

24. (canceled)

25. (canceled)

26. (canceled)


SEQUENCE LISTING (1257 sequences)

SEQ ID NO: 1 - DNA, 13 bp, synthetic construct
  source 1..13: mol_type = other DNA
tgcacgtcatgat

SEQ ID NO: 2 - DNA, 13 bp, synthetic construct
  source 1..13: mol_type = other DNA
tgcacgtgatgat

SEQ ID NO: 3 - DNA, 13 bp, synthetic construct
  source 1..13: mol_type = other DNA
tgcacgtgctgat

SEQ ID NO: 4 - DNA, 13 bp, synthetic construct
  source 1..13: mol_type = other DNA
tgcacgtgcagat

SEQ ID NO: 5 - DNA, 13 bp, synthetic construct
  source 1..13: mol_type = other DNA
tgcacgacatgat

SEQ ID NO: 6 - DNA, 13 bp, synthetic construct
  source 1..13: mol_type = other DNA
tgcacgagatgat

SEQ ID NO: 7 - DNA, 13 bp, synthetic construct
  source 1..13: mol_type = other DNA
tgcacgagctgat

SEQ ID NO: 8 - DNA, 13 bp, synthetic construct
  source 1..13: mol_type = other DNA
tgcacgagcagat

SEQ ID NO: 9 - DNA, 13 bp, synthetic construct
  source 1..13: mol_type = other DNA
tgcacgatatgat

SEQ ID NO: 10 - DNA, 13 bp, synthetic construct
  source 1..13: mol_type = other DNA
tgcacgatctgat

SEQ ID NO: 11 - DNA, 13 bp, synthetic construct
  source 1..13: mol_type = other DNA
tgcacgatcagat

SEQ ID NO: 12 - DNA, 13 bp, synthetic construct
  source 1..13: mol_type = other DNA
tgcacgatgtgat

SEQ ID NO: 13 - DNA, 13 bp, synthetic construct
  source 1..13: mol_type = other DNA
tgcacgatgagat

SEQ ID NO: 14 - DNA, 13 bp, synthetic construct
  source 1..13: mol_type = other DNA
tgcacgatgcgat

SEQ ID NO: 15 - DNA, 15 bp, synthetic construct
  source 1..15: mol_type = other DNA
tgcacgatgcatgat

SEQ ID NO: 16 - DNA, 13 bp, synthetic construct
  source 1..13: mol_type = other DNA
tgcacgcgatgat

SEQ ID NO: 17 - DNA, 13 bp, synthetic construct
  source 1..13: mol_type = other DNA
tgcacgcgctgat

SEQ ID NO: 18 - DNA, 13 bp, synthetic construct
  source 1..13: mol_type = other DNA
tgcacgcgcagat

SEQ ID NO: 19 - DNA, 13 bp, synthetic construct
  source 1..13: mol_type = other DNA
tgcacgctatgat

SEQ ID NO: 20 - DNA, 13 bp, synthetic construct
  source 1..13: mol_type = other DNA
tgcacgctctgat

SEQ ID NO: 21 - DNA, 13 bp, synthetic construct
  source 1..13: mol_type = other DNA
tgcacgctcagat

SEQ ID NO: 22 - DNA, 13 bp, synthetic construct
  source 1..13: mol_type = other DNA
tgcacgctgtgat

SEQ ID NO: 23 - DNA, 13 bp, synthetic construct
  source 1..13: mol_type = other DNA
tgcacgctgagat

SEQ ID NO: 24 - DNA, 13 bp, synthetic construct
  source 1..13: mol_type = other DNA
tgcacgctgcgat

SEQ ID NO: 25 - DNA, 15 bp, synthetic construct
  source 1..15: mol_type = other DNA
tgcacgctgcatgat

SEQ ID NO: 26 - DNA, 13 bp, synthetic construct
  source 1..13: mol_type = other DNA
tgcacgcactgat

SEQ ID NO: 27 - DNA, 13 bp, synthetic construct
  source 1..13: mol_type = other DNA
tgcacgcacagat

SEQ ID NO: 28 - DNA, 13 bp, synthetic construct
  source 1..13: mol_type = other DNA
tgcacgcagtgat

SEQ ID NO: 29 - DNA, 13 bp, synthetic construct
  source 1..13: mol_type = other DNA
tgcacgcagagat

SEQ ID NO: 30 - DNA, 13 bp, synthetic construct
  source 1..13: mol_type = other DNA
tgcacgcagcgat

SEQ ID NO: 31 - DNA, 15 bp, synthetic construct
  source 1..15: mol_type = other DNA
tgcacgcagcatgat

SEQ ID NO: 32 - DNA, 13 bp, synthetic construct
  source 1..13: mol_type = other DNA
tgcacgcatagat

SEQ ID NO: 33 - DNA, 13 bp, synthetic construct
  source 1..13: mol_type = other DNA
tgcacgcatcgat

SEQ ID NO: 34 - DNA, 15 bp, synthetic construct
  source 1..15: mol_type = other DNA
tgcacgcatcatgat

SEQ ID NO: 35 - DNA, 15 bp, synthetic construct
  source 1..15: mol_type = other DNA
tgcacgcatgatgat

SEQ ID NO: 36 - DNA, 15 bp, synthetic construct
  source 1..15: mol_type = other DNA
tgcacgcatgctgat

SEQ ID NO: 37 - DNA, 15 bp, synthetic construct
  source 1..15: mol_type = other DNA
tgcacgcatgcagat

SEQ ID NO: 38 - DNA, 13 bp, synthetic construct
  source 1..13: mol_type = other DNA
tgcactacatgat

SEQ ID NO: 39 - DNA, 13 bp, synthetic construct
  source 1..13: mol_type = other DNA
tgcactagatgat

SEQ ID NO: 40 - DNA, 13 bp, synthetic construct
  source 1..13: mol_type = other DNA
tgcactagctgat

SEQ ID NO: 41 - DNA, 13 bp, synthetic construct
  source 1..13: mol_type = other DNA
tgcactagcagat

SEQ ID NO: 42 - DNA, 13 bp, synthetic construct
  source 1..13: mol_type = other DNA
tgcactatatgat

SEQ ID NO: 43 - DNA, 13 bp, synthetic construct
  source 1..13: mol_type = other DNA
tgcactatctgat

SEQ ID NO: 44 - DNA, 13 bp, synthetic construct
  source 1..13: mol_type = other DNA
tgcactatcagat

SEQ ID NO: 45 - DNA, 13 bp, synthetic construct
  source 1..13: mol_type = other DNA
tgcactatgtgat

SEQ ID NO: 46 - DNA, 13 bp, synthetic construct
  source 1..13: mol_type = other DNA
tgcactatgagat

SEQ ID NO: 47 - DNA, 13 bp, synthetic construct
  source 1..13: mol_type = other DNA
tgcactatgcgat

SEQ ID NO: 48 - DNA, 15 bp, synthetic construct
  source 1..15: mol_type = other DNA
tgcactatgcatgat

SEQ ID NO: 49 - DNA, 13 bp, synthetic construct
  source 1..13: mol_type = other DNA
tgcactcgatgat

SEQ ID NO: 50 - DNA, 13 bp, synthetic construct
  source 1..13: mol_type = other DNA
tgcactcgctgat

SEQ ID NO: 51 - DNA, 13 bp, synthetic construct
  source 1..13: mol_type = other DNA
tgcactcgcagat

SEQ ID NO: 52 - DNA, 13 bp, synthetic construct
  source 1..13: mol_type = other DNA
tgcactctatgat

SEQ ID NO: 53 - DNA, 13 bp, synthetic construct
  source 1..13: mol_type = other DNA
tgcactctctgat

SEQ ID NO: 54 - DNA, 13 bp, synthetic construct
  source 1..13: mol_type = other DNA
tgcactctcagat

SEQ ID NO: 55 - DNA, 13 bp, synthetic construct
  source 1..13: mol_type = other DNA
tgcactctgtgat

SEQ ID NO: 56 - DNA, 13 bp, synthetic construct
  source 1..13: mol_type = other DNA
tgcactctgagat

SEQ ID NO: 57 - DNA, 13 bp, synthetic construct
  source 1..13: mol_type = other DNA
tgcactctgcgat

SEQ ID NO: 58 - DNA, 15 bp, synthetic construct
  source 1..15: mol_type = other DNA
tgcactctgcatgat

SEQ ID NO: 59 - DNA, 13 bp, synthetic construct
  source 1..13: mol_type = other DNA
tgcactcactgat

SEQ ID NO: 60 - DNA, 13 bp, synthetic construct
  source 1..13: mol_type = other DNA
tgcactcacagat

SEQ ID NO: 61 - DNA, 13 bp, synthetic construct
  source 1..13: mol_type = other DNA
tgcactcagtgat

SEQ ID NO: 62 - DNA, 13 bp, synthetic construct
  source 1..13: mol_type = other DNA
tgcactcagagat

SEQ ID NO: 63 - DNA, 13 bp, synthetic construct
  source 1..13: mol_type = other DNA
tgcactcagcgat

SEQ ID NO: 64 - DNA, 15 bp, synthetic construct
  source 1..15: mol_type = other DNA
tgcactcagcatgat

SEQ ID NO: 65 - DNA, 13 bp, synthetic construct
  source 1..13: mol_type = other DNA
tgcactcatagat

SEQ ID NO: 66 - DNA, 13 bp, synthetic construct
  source 1..13: mol_type = other DNA
tgcactcatcgat

SEQ ID NO: 67 - DNA, 15 bp, synthetic construct
  source 1..15: mol_type = other DNA
tgcactcatcatgat

SEQ ID NO: 68 - DNA, 15 bp, synthetic construct
  source 1..15: mol_type = other DNA
tgcactcatgatgat

SEQ ID NO: 69 - DNA, 15 bp, synthetic construct
  source 1..15: mol_type = other DNA
tgcactcatgctgat

SEQ ID NO: 70 - DNA, 15 bp, synthetic construct
  source 1..15: mol_type = other DNA
tgcactcatgcagat

SEQ ID NO: 71 - DNA, 13 bp, synthetic construct
  source 1..13: mol_type = other DNA
tgcactgtatgat

SEQ ID NO: 72 - DNA, 13 bp, synthetic construct
  source 1..13: mol_type = other DNA
tgcactgtctgat

SEQ ID NO: 73 - DNA, 13 bp, synthetic construct
  source 1..13: mol_type = other DNA
tgcactgtcagat

SEQ ID NO: 74 - DNA, 13 bp, synthetic construct
  source 1..13: mol_type = other DNA
tgcactgtgtgat

SEQ ID NO: 75 - DNA, 13 bp, synthetic construct
  source 1..13: mol_type = other DNA
tgcactgtgagat

SEQ ID NO: 76 - DNA, 13 bp, synthetic construct
  source 1..13: mol_type = other DNA
tgcactgtgcgat

SEQ ID NO: 77 - DNA, 15 bp, synthetic construct
  source 1..15: mol_type = other DNA
tgcactgtgcatgat

SEQ ID NO: 78 - DNA, 13 bp, synthetic construct
  source 1..13: mol_type = other DNA
tgcactgactgat

SEQ ID NO: 79 - DNA, 13 bp, synthetic construct
  source 1..13: mol_type = other DNA
tgcactgacagat

SEQ ID NO: 80 - DNA, 13 bp, synthetic construct
  source 1..13: mol_type = other DNA
tgcactgagtgat

SEQ ID NO: 81 - DNA, 13 bp, synthetic construct
  source 1..13: mol_type = other DNA
tgcactgagagat

SEQ ID NO: 82 - DNA, 13 bp, synthetic construct
  source 1..13: mol_type = other DNA
tgcactgagcgat

SEQ ID NO: 83 - DNA, 15 bp, synthetic construct
  source 1..15: mol_type = other DNA
tgcactgagcatgat

SEQ ID NO: 84 - DNA, 13 bp, synthetic construct
  source 1..13: mol_type = other DNA
tgcactgatagat

SEQ ID NO: 85 - DNA, 13 bp, synthetic construct
  source 1..13: mol_type = other DNA
tgcactgatcgat

SEQ ID NO: 86 - DNA, 15 bp, synthetic construct
  source 1..15: mol_type = other DNA
tgcactgatcatgat

SEQ ID NO: 87 - DNA, 15 bp, synthetic construct
  source 1..15: mol_type = other DNA
tgcactgatgatgat

SEQ ID NO: 88 - DNA, 15 bp, synthetic construct
  source 1..15: mol_type = other DNA
tgcactgatgctgat

SEQ ID NO: 89 - DNA, 15 bp, synthetic construct
  source 1..15: mol_type = other DNA
tgcactgatgcagat

SEQ ID NO: 90 - DNA, 13 bp, synthetic construct
  source 1..13: mol_type = other DNA
tgcactgcgtgat

SEQ ID NO: 91 - DNA, 13 bp, synthetic construct
  source 1..13: mol_type = other DNA
tgcactgcgagat

SEQ ID NO: 92 - DNA, 13 bp, synthetic construct
  source 1..13: mol_type = other DNA
tgcactgcgcgat

SEQ ID NO: 93 - DNA, 15 bp, synthetic construct
  source 1..15: mol_type = other DNA
tgcactgcgcatgat

SEQ ID NO: 94 - DNA, 13 bp, synthetic construct
  source 1..13: mol_type = other DNA
tgcactgctagat

SEQ ID NO: 95 - DNA, 13 bp, synthetic construct
  source 1..13: mol_type = other DNA
tgcactgctcgat

SEQ ID NO: 96 - DNA, 15 bp, synthetic construct
  source 1..15: mol_type = other DNA
tgcactgctcatgat

SEQ ID NO: 97 - DNA, 15 bp, synthetic construct
  source 1..15: mol_type = other DNA
tgcactgctgatgat

SEQ ID NO: 98 - DNA, 15 bp, synthetic construct
  source 1..15: mol_type = other DNA
tgcactgctgctgat

SEQ ID NO: 99 - DNA, 15 bp, synthetic construct
  source 1..15: mol_type = other DNA
tgcactgctgcagat

SEQ ID NO: 100 - DNA, 13 bp, synthetic construct
  source 1..13: mol_type = other DNA
tgcactgcacgat

SEQ ID NO: 101 - DNA, 15 bp, synthetic construct
  source 1..15: mol_type = other DNA
tgcactgcacatgat

SEQ ID NO: 102 - DNA, 15 bp, synthetic construct
  source 1..15: mol_type = other DNA
tgcactgcagatgat

SEQ ID NO: 103 - DNA, 15 bp, synthetic construct
  source 1..15: mol_type = other DNA
tgcactgcagctgat

SEQ ID NO: 104 - DNA, 15 bp, synthetic construct
  source 1..15: mol_type = other DNA
tgcactgcagcagat

SEQ ID NO: 105 - DNA, 15 bp, synthetic construct
  source 1..15: mol_type = other DNA
tgcactgcatatgat

SEQ ID NO: 106 - DNA, 15 bp, synthetic construct
  source 1..15: mol_type = other DNA
tgcactgcatctgat

SEQ ID NO: 107 - DNA, 15 bp, synthetic construct
  source 1..15: mol_type = other DNA
tgcactgcatcagat

SEQ ID NO: 108 - DNA, 15 bp, synthetic construct
  source 1..15: mol_type = other DNA
tgcactgcatgtgat

SEQ ID NO: 109 - DNA, 15 bp, synthetic construct
  source 1..15: mol_type = other DNA
tgcactgcatgagat

SEQ ID NO: 110 - DNA, 15 bp, synthetic construct
  source 1..15: mol_type = other DNA
tgcactgcatgcgat

SEQ ID NO: 111 - DNA, 13 bp, synthetic construct
  source 1..13: mol_type = other DNA
tgcacacgatgat

SEQ ID NO: 112 - DNA, 13 bp, synthetic construct
  source 1..13: mol_type = other DNA
tgcacacgctgat

SEQ ID NO: 113 - DNA, 13 bp, synthetic construct
  source 1..13: mol_type = other DNA
tgcacacgcagat

SEQ ID NO: 114 - DNA, 13 bp, synthetic construct
  source 1..13: mol_type = other DNA
tgcacactatgat

SEQ ID NO: 115 - DNA, 13 bp, synthetic construct
  source 1..13: mol_type = other DNA
tgcacactctgat

SEQ ID NO: 116 - DNA, 13 bp, synthetic construct
  source 1..13: mol_type = other DNA
tgcacactcagat

SEQ ID NO: 117 - DNA, 13 bp, synthetic construct
  source 1..13: mol_type = other DNA
tgcacactgtgat

SEQ ID NO: 118 - DNA, 13 bp, synthetic construct
  source 1..13: mol_type = other DNA
tgcacactgagat

SEQ ID NO: 119 - DNA, 13 bp, synthetic construct
  source 1..13: mol_type = other DNA
tgcacactgcgat

SEQ ID NO: 120 - DNA, 15 bp, synthetic construct
  source 1..15: mol_type = other DNA
tgcacactgcatgat

SEQ ID NO: 121 - DNA, 13 bp, synthetic construct
  source 1..13: mol_type = other DNA
tgcacacactgat

SEQ ID NO: 122 - DNA, 13 bp, synthetic construct
  source 1..13: mol_type = other DNA
tgcacacacagat

SEQ ID NO: 123 - DNA, 13 bp, synthetic construct
  source 1..13: mol_type = other DNA
tgcacacagtgat

SEQ ID NO: 124 - DNA, 13 bp, synthetic construct
  source 1..13: mol_type = other DNA
tgcacacagagat

SEQ ID NO: 125 - DNA, 13 bp, synthetic construct
  source 1..13: mol_type = other DNA
tgcacacagcgat

SEQ ID NO: 126 - DNA, 15 bp, synthetic construct
  source 1..15: mol_type = other DNA
tgcacacagcatgat

SEQ ID NO: 127 - DNA, 13 bp, synthetic construct
  source 1..13: mol_type = other DNA
tgcacacatagat

SEQ ID NO: 128 - DNA, 13 bp, synthetic construct
  source 1..13: mol_type = other DNA
tgcacacatcgat

SEQ ID NO: 129 - DNA, 15 bp, synthetic construct
  source 1..15: mol_type = other DNA
tgcacacatcatgat

SEQ ID NO: 130 - DNA, 15 bp, synthetic construct
  source 1..15: mol_type = other DNA
tgcacacatgatgat

SEQ ID NO: 131 - DNA, 15 bp, synthetic construct
  source 1..15: mol_type = other DNA
tgcacacatgctgat

SEQ ID NO: 132 - DNA, 15 bp, synthetic construct
  source 1..15: mol_type = other DNA
tgcacacatgcagat

SEQ ID NO: 133 - DNA, 13 bp, synthetic construct
  source 1..13: mol_type = other DNA
tgcacagtatgat

SEQ ID NO: 134 - DNA, 13 bp, synthetic construct
  source 1..13: mol_type = other DNA
tgcacagtctgat

SEQ ID NO: 135 - DNA, 13 bp, synthetic construct
  source 1..13: mol_type = other DNA
tgcacagtcagat

SEQ ID NO: 136 - DNA, 13 bp, synthetic construct
  source 1..13: mol_type = other DNA
tgcacagtgtgat

SEQ ID NO: 137 - DNA, 13 bp, synthetic construct
  source 1..13: mol_type = other DNA
tgcacagtgagat

SEQ ID NO: 138 - DNA, 13 bp, synthetic construct
  source 1..13: mol_type = other DNA
tgcacagtgcgat

SEQ ID NO: 139 - DNA, 15 bp, synthetic construct
  source 1..15: mol_type = other DNA
tgcacagtgcatgat

SEQ ID NO: 140 - DNA, 13 bp, synthetic construct
  source 1..13: mol_type = other DNA
tgcacagactgat

SEQ ID NO: 141 - DNA, 13 bp, synthetic construct
  source 1..13: mol_type = other DNA
tgcacagacagat

SEQ ID NO: 142 - DNA, 13 bp, synthetic construct
  source 1..13: mol_type = other DNA
tgcacagagtgat

SEQ ID NO: 143 - DNA, 13 bp, synthetic construct
  source 1..13: mol_type = other DNA
tgcacagagagat

SEQ ID NO: 144 - DNA, 13 bp, synthetic construct
  source 1..13: mol_type = other DNA
tgcacagagcgat

SEQ ID NO: 145 - DNA, 15 bp, synthetic construct
  source 1..15: mol_type = other DNA
tgcacagagcatgat

SEQ ID NO: 146 - DNA, 13 bp, synthetic construct
  source 1..13: mol_type = other DNA
tgcacagatagat

SEQ ID NO: 147 - DNA, 13 bp, synthetic construct
  source 1..13: mol_type = other DNA
tgcacagatcgat

SEQ ID NO: 148 - DNA, 15 bp, synthetic construct
  source 1..15: mol_type = other DNA
tgcacagatcatgat

SEQ ID NO: 149 - DNA, 15 bp, synthetic construct
  source 1..15: mol_type = other DNA
tgcacagatgatgat

SEQ ID NO: 150 - DNA, 15 bp, synthetic construct
  source 1..15: mol_type = other DNA
tgcacagatgctgat

SEQ ID NO: 151 - DNA, 15 bp, synthetic construct
  source 1..15: mol_type = other DNA
tgcacagatgcagat

SEQ ID NO: 152 - DNA, 13 bp, synthetic construct
  source 1..13: mol_type = other DNA
tgcacagcgtgat

SEQ ID NO: 153 - DNA, 13 bp, synthetic construct
  source 1..13: mol_type = other DNA
tgcacagcgagat

SEQ ID NO: 154 - DNA, 13 bp, synthetic construct
  source 1..13: mol_type = other DNA
tgcacagcgcgat

SEQ ID NO: 155 - DNA, 15 bp, synthetic construct
  source 1..15: mol_type = other DNA
tgcacagcgcatgat

SEQ ID NO: 156 - DNA, 13 bp, synthetic construct
  source 1..13: mol_type = other DNA
tgcacagctagat

SEQ ID NO: 157 - DNA, 13 bp, synthetic construct
  source 1..13: mol_type = other DNA
tgcacagctcgat

SEQ ID NO: 158 - DNA, 15 bp, synthetic construct
  source 1..15: mol_type = other DNA
tgcacagctcatgat

SEQ ID NO: 159 - DNA, 15 bp, synthetic construct
  source 1..15: mol_type = other DNA
tgcacagctgatgat

SEQ ID NO: 160 - DNA, 15 bp, synthetic construct
  source 1..15: mol_type = other DNA
tgcacagctgctgat

SEQ ID NO: 161 - DNA, 15 bp, synthetic construct
  source 1..15: mol_type = other DNA
tgcacagctgcagat

SEQ ID NO: 162 - DNA, 13 bp, synthetic construct
  source 1..13: mol_type = other DNA
tgcacagcacgat

SEQ ID NO: 163 - DNA, 15 bp, synthetic construct
  source 1..15: mol_type = other DNA
tgcacagcacatgat

SEQ ID NO: 164 - DNA, 15 bp, synthetic construct
  source 1..15: mol_type = other DNA
tgcacagcagatgat

SEQ ID NO: 165 - DNA, 15 bp, synthetic construct
  source 1..15: mol_type = other DNA
tgcacagcagctgat

SEQ ID NO: 166 - DNA, 15 bp, synthetic construct
  source 1..15: mol_type = other DNA
tgcacagcagcagat

SEQ ID NO: 167 - DNA, 15 bp, synthetic construct
  source 1..15: mol_type = other DNA
tgcacagcatatgat

SEQ ID NO: 168 - DNA, 15 bp, synthetic construct
  source 1..15: mol_type = other DNA
tgcacagcatctgat

SEQ ID NO: 169 - DNA, 15 bp, synthetic construct
  source 1..15: mol_type = other DNA
tgcacagcatcagat

SEQ ID NO: 170 - DNA, 15 bp, synthetic construct
  source 1..15: mol_type = other DNA
tgcacagcatgtgat

SEQ ID NO: 171 - DNA, 15 bp, synthetic construct
  source 1..15: mol_type = other DNA
tgcacagcatgagat

SEQ ID NO: 172 - DNA, 15 bp, synthetic construct
  source 1..15: mol_type = other DNA
tgcacagcatgcgat

SEQ ID NO: 173 - DNA, 13 bp, synthetic construct
  source 1..13: mol_type = other DNA
tgcacatactgat

SEQ ID NO: 174 - DNA, 13 bp, synthetic construct
  source 1..13: mol_type = other DNA
tgcacatacagat

SEQ ID NO: 175 - DNA, 13 bp, synthetic construct
  source 1..13: mol_type = other DNA
tgcacatagtgat

SEQ ID NO: 176 - DNA, 13 bp, synthetic construct
  source 1..13: mol_type = other DNA
tgcacatagagat

SEQ ID NO: 177 - DNA, 13 bp, synthetic construct
  source 1..13: mol_type = other DNA
tgcacatagcgat

SEQ ID NO: 178 - DNA, 15 bp, synthetic construct
  source 1..15: mol_type = other DNA
tgcacatagcatgat

SEQ ID NO: 179 - DNA, 13 bp, synthetic construct
  source 1..13: mol_type = other DNA
tgcacatatagat

SEQ ID NO: 180 - DNA, 13 bp, synthetic construct
  source 1..13: mol_type = other DNA
tgcacatatcgat

SEQ ID NO: 181 - DNA, 15 bp, synthetic construct
  source 1..15: mol_type = other DNA
tgcacatatcatgat

SEQ ID NO: 182 - DNA, 15 bp, synthetic construct
  source 1..15: mol_type = other DNA
tgcacatatgatgat

SEQ ID NO: 183 - DNA, 15 bp, synthetic construct
  source 1..15: mol_type = other DNA
tgcacatatgctgat

SEQ ID NO: 184 - DNA, 15 bp, synthetic construct
  source 1..15: mol_type = other DNA
tgcacatatgcagat

SEQ ID NO: 185 - DNA, 13 bp, synthetic construct
  source 1..13: mol_type = other DNA
tgcacatcgtgat

SEQ ID NO: 186 - DNA, 13 bp, synthetic construct
  source 1..13: mol_type = other DNA
tgcacatcgagat

SEQ ID NO: 187 - DNA, 13 bp, synthetic construct
  source 1..13: mol_type = other DNA
tgcacatcgcgat

SEQ ID NO: 188 - DNA, 15 bp, synthetic construct
  source 1..15: mol_type = other DNA
tgcacatcgcatgat

SEQ ID NO: 189 - DNA, 13 bp, synthetic construct
  source 1..13: mol_type = other DNA
tgcacatctagat

SEQ ID NO: 190 - DNA, 13 bp, synthetic construct
  source 1..13: mol_type = other DNA
tgcacatctcgat

SEQ ID NO: 191 - DNA, 15 bp, synthetic construct
  source 1..15: mol_type = other DNA
tgcacatctcatgat

SEQ ID NO: 192 - DNA, 15 bp, synthetic construct
  source 1..15: mol_type = other DNA
tgcacatctgatgat

SEQ ID NO: 193 - DNA, 15 bp, synthetic construct
  source 1..15: mol_type = other DNA
tgcacatctgctgat

SEQ ID NO: 194 - DNA, 15 bp, synthetic construct
  source 1..15: mol_type = other DNA
tgcacatctgcagat

SEQ ID NO: 195 - DNA, 13 bp, synthetic construct
  source 1..13: mol_type = other DNA
tgcacatcacgat

SEQ ID NO: 196 - DNA, 15 bp, synthetic construct
  source 1..15: mol_type = other DNA
tgcacatcacatgat

SEQ ID NO: 197 - DNA, 15 bp, synthetic construct
  source 1..15: mol_type = other DNA
tgcacatcagatgat

SEQ ID NO: 198 - DNA, 15 bp, synthetic construct
  source 1..15: mol_type = other DNA
tgcacatcagctgat

SEQ ID NO: 199 - DNA, 15 bp, synthetic construct
  source 1..15: mol_type = other DNA
tgcacatcagcagat

SEQ ID NO: 200 - DNA, 15 bp, synthetic construct
  source 1..15: mol_type = other DNA
tgcacatcatatgat

SEQ ID NO: 201 - DNA, 15 bp, synthetic construct
  source 1..15: mol_type = other DNA
tgcacatcatctgat

SEQ ID NO: 202 - DNA, 15 bp, synthetic construct
  source 1..15: mol_type = other DNA
tgcacatcatcagat

SEQ ID NO: 203 - DNA, 15 bp, synthetic construct
  source 1..15: mol_type = other DNA
tgcacatcatgtgat

SEQ ID NO: 204 - DNA, 15 bp, synthetic construct
  source 1..15: mol_type = other DNA
tgcacatcatgagat

SEQ ID NO: 205 - DNA, 15 bp, synthetic construct
  source 1..15: mol_type = other DNA
tgcacatcatgcgat

SEQ ID NO: 206 - DNA, 13 bp, synthetic construct
  source 1..13: mol_type = other DNA
tgcacatgtagat

SEQ ID NO: 207 - DNA, 13 bp, synthetic construct
  source 1..13: mol_type = other DNA
tgcacatgtcgat

SEQ ID NO: 208 - DNA, 15 bp, synthetic construct
  source 1..15: mol_type = other DNA
tgcacatgtcatgat

SEQ ID NO: 209 - DNA, 15 bp, synthetic construct
  source 1..15: mol_type = other DNA
tgcacatgtgatgat

SEQ ID NO: 210 - DNA, 15 bp, synthetic construct
  source 1..15: mol_type = other DNA
tgcacatgtgctgat

SEQ ID NO: 211 - DNA, 15 bp, synthetic construct
  source 1..15: mol_type = other DNA
tgcacatgtgcagat

SEQ ID NO: 212 - DNA, 13 bp, synthetic construct
  source 1..13: mol_type = other DNA
tgcacatgacgat

SEQ ID NO: 213 - DNA, 15 bp, synthetic construct
  source 1..15: mol_type = other DNA
tgcacatgacatgat

SEQ ID NO: 214 - DNA, 15 bp, synthetic construct
  source 1..15: mol_type = other DNA
tgcacatgagatgat

SEQ ID NO: 215 - DNA, 15 bp, synthetic construct
  source 1..15: mol_type = other DNA
tgcacatgagctgat

SEQ ID NO: 216 - DNA, 15 bp, synthetic construct
  source 1..15: mol_type = other DNA
tgcacatgagcagat

SEQ ID NO: 217 - DNA, 15 bp, synthetic construct
  source 1..15: mol_type = other DNA
tgcacatgatatgat

SEQ ID NO: 218 - DNA, 15 bp, synthetic construct
  source 1..15: mol_type = other DNA
tgcacatgatctgat

SEQ ID NO: 219 - DNA, 15 bp, synthetic construct
  source 1..15: mol_type = other DNA
tgcacatgatcagat

SEQ ID NO: 220 - DNA, 15 bp, synthetic construct
  source 1..15: mol_type = other DNA
tgcacatgatgtgat

SEQ ID NO: 221 - DNA, 15 bp, synthetic construct
  source 1..15: mol_type = other DNA
tgcacatgatgagat

SEQ ID NO: 222 - DNA, 15 bp, synthetic construct
  source 1..15: mol_type = other DNA
tgcacatgatgcgat

SEQ ID NO: 223 - DNA, 15 bp, synthetic construct
  source 1..15: mol_type = other DNA
tgcacatgcgatgat

SEQ ID NO: 224 - DNA, 15 bp, synthetic construct
  source 1..15: mol_type = other DNA
tgcacatgcgctgat

SEQ ID NO: 225 - DNA, 15 bp, synthetic construct
  source 1..15: mol_type = other DNA
tgcacatgcgcagat

SEQ ID NO: 226 - DNA, 15 bp, synthetic construct
  source 1..15: mol_type = other DNA
tgcacatgctatgat

SEQ ID NO: 227 - DNA, 15 bp, synthetic construct
  source 1..15: mol_type = other DNA
tgcacatgctctgat

SEQ ID NO: 228 - DNA, 15 bp, synthetic construct
  source 1..15: mol_type = other DNA
tgcacatgctcagat

SEQ ID NO: 229 - DNA, 15 bp, synthetic construct
  source 1..15: mol_type = other DNA
tgcacatgctgtgat

SEQ ID NO: 230 - DNA, 15 bp, synthetic construct
  source 1..15: mol_type = other DNA
tgcacatgctgagat

SEQ ID NO: 231 - DNA, 15 bp, synthetic construct
  source 1..15: mol_type = other DNA
tgcacatgctgcgat

SEQ ID NO: 232 - DNA, 15 bp, synthetic construct
  source 1..15: mol_type = other DNA
tgcacatgcactgat

SEQ ID NO: 233 - DNA, 15 bp, synthetic construct
  source 1..15: mol_type = other DNA
tgcacatgcacagat

SEQ ID NO: 234 - DNA, 15 bp, synthetic construct
  source 1..15: mol_type = other DNA
tgcacatgcagtgat

SEQ ID NO: 235 - DNA, 15 bp, synthetic construct
  source 1..15: mol_type = other DNA
tgcacatgcagagat

SEQ ID NO: 236 - DNA, 15 bp, synthetic construct
  source 1..15: mol_type = other DNA
tgcacatgcagcgat

SEQ ID NO: 237 - DNA, 15 bp, synthetic construct
  source 1..15: mol_type = other DNA
tgcacatgcatagat

SEQ ID NO: 238 - DNA, 15 bp, synthetic construct
  source 1..15: mol_type = other DNA
tgcacatgcatcgat

SEQ ID NO: 239 - DNA, 19 bp, synthetic construct
  source 1..19: mol_type = other DNA
tgcacggtacatgcatgat

SEQ ID NO: 240 - DNA, 19 bp, synthetic construct
  source 1..19: mol_type = other DNA
tgcacgtaatgctcatgat

SEQ ID NO: 241 - DNA, 19 bp, synthetic construct
  source 1..19: mol_type = other DNA
tgcacgtatggcagctgat

SEQ ID NO: 242 - DNA, 19 bp, synthetic construct
  source 1..19: mol_type = other DNA
tgcacgtcgcattcatgat

SEQ ID NO: 243 - DNA, 19 bp, synthetic construct
  source 1..19: mol_type = other DNA
tgcacgtctgatgccagat

SEQ ID NO: 244 - DNA, 19 bp, synthetic construct
  source 1..19: mol_type = other DNA
tgcacggtcagcatgtgat

SEQ ID NO: 245 - DNA, 19 bp, synthetic construct
  source 1..19: mol_type = other DNA
tgcacgtccatcatatgat

SEQ ID NO: 246 - DNA, 19 bp, synthetic construct
  source 1..19: mol_type = other DNA
tgcacgtcattgcacagat

SEQ ID NO: 247 - DNA, 19 bp, synthetic construct
  source 1..19: mol_type = other DNA
tgcacgtgtgcaacatgat

SEQ ID NO: 248 - DNA, 19 bp, synthetic construct
  source 1..19: mol_type = other DNA
tgcacgtgtgcatggcgat

SEQ ID NO: 249 - DNA, 19 bp, synthetic construct
  source 1..19: mol_type = other DNA
tgcacggtgagcagatgat

SEQ ID NO: 250 - DNA, 19 bp, synthetic construct
  source 1..19: mol_type = other DNA
tgcacgtggatctgatgat

SEQ ID NO: 251 - DNA, 21 bp, synthetic construct
  source 1..21: mol_type = other DNA
tgcacgtgattgatgcatgat

SEQ ID NO: 252 - DNA, 19 bp, synthetic construct
  source 1..19: mol_type = other DNA
tgcacgtgcgcaagcagat

SEQ ID NO: 253 - DNA, 21 bp, synthetic construct
  source 1..21: mol_type = other DNA
tgcacgtgctcatggcatgat

SEQ ID NO: 254 - DNA, 19 bp, synthetic construct
  source 1..19: mol_type = other DNA
tgcacggtgctgctatgat

SEQ ID NO: 255 - DNA, 19 bp, synthetic construct
  source 1..19: mol_type = other DNA
tgcacgtggcacacatgat

SEQ ID NO: 256 - DNA, 19 bp, synthetic construct
  source 1..19: mol_type = other DNA
tgcacgtgcaacatgagat

SEQ ID NO: 257 - DNA, 19 bp, synthetic construct
  source 1..19: mol_type = other DNA
tgcacgtgcagttcatgat

SEQ ID NO: 258 - DNA, 19 bp, synthetic construct
  source 1..19: mol_type = other DNA
tgcacgtgcatagcctgat

SEQ ID NO: 259 - DNA, 19 bp, synthetic construct
  source 1..19: mol_type = other DNA
tgcacggtgcatatcagat

SEQ ID NO: 260 - DNA, 19 bp, synthetic construct
  source 1..19: mol_type = other DNA
tgcacgtggcatgtgtgat

SEQ ID NO: 261 - DNA, 21 bp, synthetic construct
  source 1..21: mol_type = other DNA
tgcacgtgcaatgcgcatgat

SEQ ID NO: 262 - DNA, 21 bp, synthetic construct
  source 1..21: mol_type = other DNA
tgcacgtgcatggcatctgat

SEQ ID NO: 263 - DNA, 19 bp, synthetic construct
  source 1..19: mol_type = other DNA
tgcacgactcatgcctgat

SEQ ID NO: 264 - DNA, 19 bp, synthetic construct
  source 1..19: mol_type = other DNA
tgcacggacagctgcagat

SEQ ID NO: 265 - DNA, 19 bp, synthetic construct
  source 1..19: mol_type = other DNA
tgcacgaccatatcatgat

SEQ ID NO: 266 - DNA, 19 bp, synthetic construct
  source 1..19: mol_type = other DNA
tgcacgacattgtgctgat

SEQ ID NO: 267 - DNA, 19 bp, synthetic construct
  source 1..19: mol_type = other DNA
tgcacgacatgaagcagat

SEQ ID NO: 268 - DNA, 19 bp, synthetic construct
  source 1..19: mol_type = other DNA
tgcacgacatgcacctgat

SEQ ID NO: 269 - DNA, 21 bp, synthetic construct
  source 1..21: mol_type = other DNA
tgcacgacatgcatgaatgat

SEQ ID NO: 270 - DNA, 21 bp, synthetic construct
  source 1..21: mol_type = other DNA
tgcacggagtgcatgcatgat

SEQ ID NO: 271 - DNA, 19 bp, synthetic construct
  source 1..19: mol_type = other DNA
tgcacgaggagcatgtgat

SEQ ID NO: 272 - DNA, 19 bp, synthetic construct
  source 1..19: mol_type = other DNA
tgcacgagattgagatgat

SEQ ID NO: 273 - DNA, 19 bp, synthetic construct
  source 1..19: mol_type = other DNA
tgcacgagatgcctcagat

SEQ ID NO: 274 - DNA, 19 bp, synthetic construct
  source 1..19: mol_type = other DNA
tgcacgagcgctcaatgat

SEQ ID NO: 275 - DNA, 19 bp, synthetic construct
  source 1..19: mol_type = other DNA
tgcacggagctatgcagat

SEQ ID NO: 276 - DNA, 19 bp, synthetic construct
  source 1..19: mol_type = other DNA
tgcacgaggctgatctgat

SEQ ID NO: 277 - DNA, 19 bp, synthetic construct
  source 1..19: mol_type = other DNA
tgcacgagcttgctgtgat

SEQ ID NO: 278 - DNA, 21 bp, synthetic construct
  source 1..21: mol_type = other DNA
tgcacgagcacaatgcatgat

SEQ ID NO: 279 - DNA, 19 bp, synthetic construct
  source 1..19: mol_type = other DNA
tgcacgagcatcaggtgat

SEQ ID NO: 280 - DNA, 21 bp, synthetic construct
  source 1..21: mol_type = other DNA
tgcacgagcatgcatggcgat

SEQ ID NO: 281 - DNA, 19 bp, synthetic construct
  source 1..19: mol_type = other DNA
tgcacggatagatgctgat

SEQ ID NO: 282 - DNA, 19 bp, synthetic construct
  source 1..19: mol_type = other DNA
tgcacgattagcatatgat

SEQ ID NO: 283 - DNA, 19 bp, synthetic construct
  source 1..19: mol_type = other DNA
tgcacgatattcgcatgat

SEQ ID NO: 284 - DNA, 19 bp, synthetic construct
  source 1..19: mol_type = other DNA
tgcacgatatcaatcagat

SEQ ID NO: 285 - DNA, 19 bp, synthetic construct
  source 1..19: mol_type = other DNA
tgcacgatatcatggtgat

SEQ ID NO: 286 - DNA, 19 bp, synthetic construct
  source 1..19: mol_type = other DNA
tgcacggatcgcatgcgat

SEQ ID NO: 287 - DNA, 19 bp, synthetic construct
  source 1..19: mol_type = other DNA
tgcacgattctgtcatgat

SEQ ID NO: 288 - DNA, 19 bp, synthetic construct
  source 1..19: mol_type = other DNA
tgcacgatcttgagctgat

SEQ ID NO: 289 - DNA, 19 bp, synthetic construct
  source 1..19: mol_type = other DNA
tgcacgatcacaagctgat

SEQ ID NO: 290 - DNA, 19 bp, synthetic construct
  source 1..19: mol_type = other DNA
tgcacgatcatatggcgat

SEQ ID NO: 291 - DNA, 19 bp, synthetic construct
  source 1..19: mol_type = other DNA
tgcacggatcatgcgtgat

SEQ ID NO: 292 - DNA, 19 bp, synthetic construct
  source 1..19: mol_type = other DNA
tgcacgattcatgctcgat

SEQ ID NO: 293 - DNA, 19 bp, synthetic construct
  source 1..19: mol_type = other DNA
tgcacgatgttgagcagat

SEQ ID NO: 294 - DNA, 19 bp, synthetic construct
  source 1..19: mol_type = other DNA
tgcacgatgtgccatagat

SEQ ID NO: 295 - DNA, 19 bp, synthetic construct
  source 1..19: mol_type = other DNA
tgcacgatgactgccagat

SEQ ID NO: 296 - DNA, 19 bp, synthetic construct
  source 1..19: mol_type = other DNA
tgcacggatgagcactgat

SEQ ID NO: 297 - DNA, 19 bp, synthetic construct
  source 1..19: mol_type = other DNA
tgcacgattgatgcgcgat

SEQ ID NO: 298 - DNA, 19 bp, synthetic construct
  source 1..19: mol_type = other DNA
tgcacgatgccgtgcagat

SEQ ID NO: 299 - DNA, 19 bp, synthetic construct
  source 1..19: mol_type = other DNA
tgcacgatgcgaatatgat

SEQ ID NO: 300 - DNA, 19 bp, synthetic construct
  source 1..19: mol_type = other DNA
tgcacgatgctctggcgat

SEQ ID NO: 301 - DNA, 19 bp, synthetic construct
  source 1..19: mol_type = other DNA
tgcacggatgctcactgat

SEQ ID NO: 302 - DNA, 21 bp, synthetic construct
  source 1..21: mol_type = other DNA
tgcacgattgctgcagatgat

SEQ ID NO: 303 - DNA, 19 bp, synthetic construct
  source 1..19: mol_type = other DNA
tgcacgatgccactgtgat

SEQ ID NO: 304 - DNA, 19 bp, synthetic construct
  source 1..19: mol_type = other DNA
tgcacgatgcaggagagat

SEQ ID NO: 305 - DNA, 19 bp, synthetic construct
  source 1..19: mol_type = other DNA
tgcacgatgcagattcgat

SEQ ID NO: 306 - DNA, 19 bp, synthetic construct
  source 1..19: mol_type = other DNA
tgcacggatgcagctagat

SEQ ID NO: 307 - DNA, 21 bp, synthetic construct
  source 1..21: mol_type = other DNA
tgcacgattgcatatgatgat

SEQ ID NO: 308 - DNA, 21 bp, synthetic construct
  source 1..21: mol_type = other DNA
tgcacgatgccatctcatgat

SEQ ID NO: 309 - DNA, 21 bp, synthetic construct
  source 1..21: mol_type = other DNA
tgcacgatgcattcagcagat

SEQ ID NO: 310 - DNA, 21 bp, synthetic construct
  source 1..21: mol_type = other DNA
tgcacgatgcatgaacatgat

SEQ ID NO: 311 - DNA, 19 bp, synthetic construct
  source 1..19: mol_type = other DNA
tgcacggcgtgcgcatgat

SEQ ID NO: 312 - DNA, 19 bp, synthetic construct
  source 1..19: mol_type = other DNA
tgcacgcggagcatcagat

SEQ ID NO: 313 - DNA, 19 bp, synthetic construct
  source 1..19: mol_type = other DNA
tgcacgcgattcatgtgat

SEQ ID NO: 314 - DNA, 19 bp, synthetic construct
  source 1..19: mol_type = other DNA
tgcacgcgatgcctctgat

SEQ ID NO: 315 - DNA, 19 bp, synthetic construct
  source 1..19: mol_type = other DNA
tgcacgcgcgcagaatgat

SEQ ID NO: 316 - DNA, 19 bp, synthetic construct
  source 1..19: mol_type = other DNA
tgcacggcgctgagctgat

SEQ ID NO: 317 - DNA, 19 bp, synthetic construct
  source 1..19: mol_type = other DNA
tgcacgcggctgatatgat

SEQ ID NO: 318 - DNA, 21 bp, synthetic construct
  source 1..21: mol_type = other DNA
tgcacgcgcttgcatgcagat

SEQ ID NO: 319 - DNA, 19 bp, synthetic construct
  source 1..19: mol_type = other DNA
tgcacgcgcacaatatgat

SEQ ID NO: 320 - DNA, 21 bp, synthetic construct
  source 1..21: mol_type = other DNA
tgcacgcgcagatggcatgat

SEQ ID NO: 321 - DNA, 19 bp, synthetic construct
  source 1..19: mol_type = other DNA
tgcacggcgcagctgtgat

SEQ ID NO: 322 - DNA, 19 bp, synthetic construct
  source 1..19: mol_type = other DNA
tgcacgcggcatacatgat

SEQ ID NO: 323 - DNA, 19 bp, synthetic construct
  source 1..19: mol_type = other DNA
tgcacgcgcaatatgcgat

SEQ ID NO: 324 - DNA, 21 bp, synthetic construct
  source 1..21: mol_type = other DNA
tgcacgcgcatcctgcatgat

SEQ ID NO: 325 - DNA, 19 bp, synthetic construct
  source 1..19: mol_type = other DNA
tgcacgcgcatgcttagat

SEQ ID NO: 326 - DNA, 19 bp, synthetic construct
  source 1..19: mol_type = other DNA
tgcacggctagatgcagat

SEQ ID NO: 327 - DNA, 19 bp, synthetic construct
  source 1..19: mol_type = other DNA
tgcacgcttagctgctgat

SEQ ID NO: 328 - DNA, 19 bp, synthetic construct
  source 1..19: mol_type = other DNA
tgcacgctattcacatgat

SEQ ID NO: 329 - DNA, 19 bp, synthetic construct
  source 1..19: mol_type = other DNA
tgcacgctatgaatatgat

SEQ ID NO: 330 - DNA, 19 bp, synthetic construct
  source 1..19: mol_type = other DNA
tgcacgctatgcgaatgat

SEQ ID NO: 331 - DNA, 19 bp, synthetic construct
  source 1..19: mol_type = other DNA
tgcacggctatgcatcgat

SEQ ID NO: 332 - DNA, 19 bp, synthetic construct
  source 1..19: mol_type = other DNA
tgcacgcttcgcgcatgat

SEQ ID NO: 333 - DNA, 19 bp, synthetic construct
  source 1..19: mol_type = other DNA
tgcacgctcggcatgagat

SEQ ID NO: 334 - DNA, 19 bp, synthetic construct
  source 1..19: mol_type = other DNA
tgcacgctctgttgatgat

SEQ ID NO: 335 - DNA, 19 bp, synthetic construct
  source 1..19: mol_type = other DNA
tgcacgctctgcacctgat

SEQ ID NO: 336 - DNA, 19 bp, synthetic construct
  source 1..19: mol_type = other DNA
tgcacggctcactcatgat

SEQ ID NO: 337 - DNA, 19 bp, synthetic construct
  source 1..19: mol_type = other DNA
tgcacgcttcacagatgat

SEQ ID NO: 338 - DNA, 19 bp, synthetic construct
  source 1..19: mol_type = other DNA
tgcacgctcaacatgcgat

SEQ ID NO: 339 - DNA, 19 bp, synthetic construct
  source 1..19: mol_type = other DNA
tgcacgctcagaagctgat

SEQ ID NO: 340 - DNA, 21 bp, synthetic construct
  source 1..21: mol_type = other DNA
tgcacgctcatatggcatgat

SEQ ID NO: 341 - DNA, 19 bp, synthetic construct
  source 1..19: mol_type = other DNA
tgcacggctcatcgctgat

SEQ ID NO: 342 - DNA, 21 bp, synthetic construct
  source 1..21: mol_type = other DNA
tgcacgcttcatgctgcagat

SEQ ID NO: 343 - DNA, 21 bp, synthetic construct
  source 1..21: mol_type = other DNA
tgcacgctgttgcatgatgat

SEQ ID NO: 344 - DNA, 19 bp, synthetic construct
  source 1..19: mol_type = other DNA
tgcacgctgagaatctgat

SEQ ID NO: 345 - DNA, 19 bp, synthetic construct
  source 1..19: mol_type = other DNA
tgcacgctgatcaggcgat

SEQ ID NO: 346 - DNA, 19 bp, synthetic construct
  source 1..19: mol_type = other DNA
tgcacggctgatcatagat

SEQ ID NO: 347 - DNA, 19 bp, synthetic construct
  source 1..19: mol_type = other DNA
tgcacgcttgcgatgtgat

SEQ ID NO: 348 - DNA, 21 bp, synthetic construct
  source 1..21: mol_type = other DNA
tgcacgctgcctgctgctgat

SEQ ID NO: 349 - DNA, 19 bp, synthetic construct
  source 1..19: mol_type = other DNA
tgcacgctgcaggcacgat

SEQ ID NO: 350 - DNA, 21 bp, synthetic construct
  source 1..21: mol_type = other DNA
tgcacgctgcatgaagatgat

SEQ ID NO: 351 - DNA, 19 bp, synthetic construct
  source 1..19: mol_type = other DNA
tgcacggcacgatgctgat

SEQ ID NO: 352 - DNA, 19 bp, synthetic construct
  source 1..19: mol_type = other DNA
tgcacgcaacgcatatgat

SEQ ID NO: 353 - DNA, 19 bp, synthetic construct
  source 1..19: mol_type = other DNA
tgcacgcacttcgcatgat

SEQ ID NO: 354 - DNA, 19 bp, synthetic construct
  source 1..19: mol_type = other DNA
tgcacgcactgttgcagat

SEQ ID NO: 355 - DNA, 19 bp, synthetic construct
  source 1..19: mol_type = other DNA
tgcacgcactgctcctgat

SEQ ID NO: 356 - DNA, 19 bp, synthetic construct
  source 1..19: mol_type = other DNA
tgcacggcactgcagtgat

SEQ ID NO: 357 - DNA, 19 bp, synthetic construct
  source 1..19: mol_type = other DNA
tgcacgcaacacatgtgat

SEQ ID NO: 358 - DNA, 19 bp, synthetic construct
  source 1..19: mol_type = other DNA
tgcacgcacaagtcatgat

SEQ ID NO: 359 - DNA, 21 bp, synthetic construct
  source 1..21: mol_type = other DNA
tgcacgcacagccagcatgat

SEQ ID NO: 360 - DNA, 19 bp, synthetic construct
  source 1..19: mol_type = other DNA
tgcacgcacatagcctgat

SEQ ID NO: 361 - DNA, 19 bp, synthetic construct
  source 1..19: mol_type = other DNA
tgcacggcacatatgagat

SEQ ID NO: 362 - DNA, 19 bp, synthetic construct
  source 1..19: mol_type = other DNA
tgcacgcaacatcatcgat

SEQ ID NO: 363 - DNA, 19 bp, synthetic construct
  source 1..19: mol_type = other DNA
tgcacgcacaatgcgagat

SEQ ID NO: 364 - DNA, 19 bp, synthetic construct
  source 1..19: mol_type = other DNA
tgcacgcagtcaagctgat

SEQ ID NO: 365 - DNA, 19 bp, synthetic construct
  source 1..19: mol_type = other DNA
tgcacgcagtcatccagat

SEQ ID NO: 366 - DNA, 21 bp, synthetic construct
  source 1..21: mol_type = other DNA
tgcacgcagagctgcaatgat

SEQ ID NO: 367 - DNA, 19 bp, synthetic construct
  source 1..19: mol_type = other DNA
tgcacggcagagcagcgat

SEQ ID NO: 368 - DNA, 21 bp, synthetic construct
  source 1..21: mol_type = other DNA
tgcacgcaagatatgcatgat

SEQ ID NO: 369 - DNA, 19 bp, synthetic construct
  source 1..19: mol_type = other DNA
tgcacgcagaatgcgtgat

SEQ ID NO: 370 - DNA, 21 bp, synthetic construct
  source 1..21: mol_type = other DNA
tgcacgcagatggcacatgat

SEQ ID NO: 371 - DNA, 21 bp, synthetic construct
  source 1..21: mol_type = other DNA
tgcacgcagatgcaatgagat

SEQ ID NO: 372 - DNA, 21 bp, synthetic construct
  source 1..21: mol_type = other DNA
tgcacgcagctcatgaatgat

SEQ ID NO: 373 - DNA, 19 bp, synthetic construct
  source 1..19: mol_type = other DNA
tgcacggcagcagactgat

SEQ ID NO: 374 - DNA, 19 bp, synthetic construct
  source 1..19: mol_type = other DNA
tgcacgcaagcatgtcgat

SEQ ID NO: 375 - DNA, 21 bp, synthetic construct
  source 1..21: mol_type = other DNA
tgcacgcagccatgtgatgat

SEQ ID NO: 376 - DNA, 19 bp, synthetic construct
  source 1..19: mol_type = other DNA
tgcacgcatacttgatgat

SEQ ID NO: 377 - DNA, 19 bp, synthetic construct
  source 1..19: mol_type = other DNA
tgcacgcatacatcctgat

SEQ ID NO: 378 - DNA, 19 bp, synthetic construct
  source 1..19: mol_type = other DNA
tgcacggcatagagatgat

SEQ ID NO: 379 - DNA, 19 bp, synthetic construct
  source 1..19: mol_type = other DNA
tgcacgcaatagctcagat

SEQ ID NO: 380 - DNA, 19 bp, synthetic construct
  source 1..19: mol_type = other DNA
tgcacgcataatgtctgat

SEQ ID NO: 381 - DNA, 21 bp, synthetic construct
  source 1..21: mol_type = other DNA
tgcacgcatatggcagcagat

SEQ ID NO: 382 - DNA, 19 bp, synthetic construct
  source 1..19: mol_type = other DNA
tgcacgcatcgagccagat

SEQ ID NO: 383 - DNA, 19 bp, synthetic construct
  source 1..19: mol_type = other DNA
tgcacggcatcgctgtgat

SEQ ID NO: 384 - DNA, 19 bp, synthetic construct
  source 1..19: mol_type = other DNA
tgcacgcaatctatctgat

SEQ ID NO: 385 - DNA, 19 bp, synthetic construct
  source 1..19: mol_type = other DNA
tgcacgcatcctcacagat

SEQ ID NO: 386 - DNA, 19 bp, synthetic construct
  source 1..19: mol_type = other DNA
tgcacgcatctggcgcgat

SEQ ID NO: 387 - DNA, 19 bp, synthetic construct
  source 1..19: mol_type = other DNA
tgcacgcatcacattagat

SEQ ID NO: 388 - DNA, 19 bp, synthetic construct
  source 1..19: mol_type = other DNA
tgcacggcatcagtgagat

SEQ ID NO: 389 - DNA, 21 bp, synthetic construct
  source 1..21: mol_type = other DNA
tgcacgcaatcatgatcagat

SEQ ID NO: 390 - DNA, 21 bp, synthetic construct
  source 1..21: mol_type = other DNA
tgcacgcatccatgatgtgat

SEQ ID NO: 391 - DNA, 21 bp, synthetic construct
  source 1..21: mol_type = other DNA
tgcacgcatcattgctatgat

SEQ ID NO: 392 - DNA, 19 bp, synthetic construct
  source 1..19: mol_type = other DNA
tgcacgcatgtatggcgat

SEQ ID NO: 393 - DNA, 19 bp, synthetic construct
  source 1..19: mol_type = other DNA
tgcacggcatgtctgtgat

SEQ ID NO: 394 - DNA, 21 bp, synthetic construct
  source 1..21: mol_type = other DNA
tgcacgcaatgtgtgcatgat

SEQ ID NO: 395 - DNA, 19 bp, synthetic construct
  source 1..19: mol_type = other DNA
tgcacgcatggtgactgat

SEQ ID NO: 396 - DNA, 19 bp, synthetic construct
  source 1..19: mol_type = other DNA
tgcacgcatgtggctcgat

SEQ ID NO: 397 - DNA, 19 bp, synthetic construct
  source 1..19: mol_type = other DNA
tgcacgcatgacaccagat

SEQ ID NO: 398 - DNA, 19 bp, synthetic construct
  source 1..19: mol_type = other DNA
tgcacggcatgacagtgat

SEQ ID NO: 399 - DNA, 19 bp, synthetic construct
  source 1..19: mol_type = other DNA
tgcacgcaatgagtgtgat

SEQ ID NO: 400 - DNA, 19 bp, synthetic construct
  source 1..19: mol_type = other DNA
tgcacgcatggcgcgagat

SEQ ID NO: 401 - DNA, 21 bp, synthetic construct
  source 1..21: mol_type = other DNA
tgcacgcatgcggcacatgat

SEQ ID NO: 402 - DNA, 19 bp, synthetic construct
  source 1..19: mol_type = other DNA
tgcacgcatgctaggtgat

SEQ ID NO: 403 - DNA, 19 bp, synthetic construct
  source 1..19: mol_type = other DNA
tgcacggcatgctgtagat

SEQ ID NO: 404 - DNA, 21 bp, synthetic construct
  source 1..21: mol_type = other DNA
tgcacgcaatgcacgcatgat

SEQ ID NO: 405 - DNA, 21 bp, synthetic construct
  source 1..21: mol_type = other DNA
tgcacgcatggcagctctgat

SEQ ID NO: 406 - DNA, 19 bp, synthetic construct
  source 1..19: mol_type = other DNA
tgcacgcatgcaatacgat

SEQ ID NO: 407 - DNA, 21 bp, synthetic construct
  source 1..21: mol_type = other DNA
tgcacgcatgcatcctgagat

SEQ ID NO: 408 - DNA, 19 bp, synthetic construct
  source 1..19: mol_type = other DNA
tgcacttacgcatcatgat

SEQ ID NO: 409 - DNA, 19 bp, synthetic construct
  source 1..19: mol_type = other DNA
tgcactacctgatgcagat

SEQ ID NO: 410 - DNA, 19 bp, synthetic construct
  source 1..19: mol_type = other DNA
tgcactactggcagctgat

SEQ ID NO: 411 - DNA, 19 bp, synthetic construct
  source 1..19: mol_type = other DNA
tgcactacagcaatgtgat

SEQ ID NO: 412 - DNA, 21 bp, synthetic construct
  source 1..21: mol_type = other DNA
tgcactacatcatggcatgat

SEQ ID NO: 413 - DNA, 19 bp, synthetic construct
  source 1..19: mol_type = other DNA
tgcacttacatgtcatgat

SEQ ID NO: 414 - DNA, 19 bp, synthetic construct
  source 1..19: mol_type = other DNA
tgcactaccatgctgagat

SEQ ID NO: 415 - DNA, 19 bp, synthetic construct
  source 1..19: mol_type = other DNA
tgcactacattgcacagat

SEQ ID NO: 416 - DNA, 19 bp, synthetic construct
  source 1..19: mol_type = other DNA
tgcactagtgcaacatgat

SEQ ID NO: 417 - DNA, 19 bp, synthetic construct
  source 1..19: mol_type = other DNA
tgcactagtgcatggtgat

SEQ ID NO: 418 - DNA, 21 bp, synthetic construct
  source 1..21: mol_type = other DNA
tgcactagagcatgcaatgat

SEQ ID NO: 419 - DNA, 19 bp, synthetic construct
  source 1..19: mol_type = other DNA
tgcacttagatatcatgat

SEQ ID NO: 420 - DNA, 19 bp, synthetic construct
  source 1..19: mol_type = other DNA
tgcactaggatcatgcgat

SEQ ID NO: 421 - DNA, 19 bp, synthetic construct
  source 1..19: mol_type = other DNA
tgcactagattgcgcagat

SEQ ID NO: 422 - DNA, 19 bp, synthetic construct
  source 1..19: mol_type = other DNA
tgcactagcgcaatgagat

SEQ ID NO: 423 - DNA, 19 bp, synthetic construct
  source 1..19: mol_type = other DNA
tgcactagctcagccagat

SEQ ID NO: 424 - DNA, 19 bp, synthetic construct
  source 1..19: mol_type = other DNA
tgcacttagctgtgatgat

SEQ ID NO: 425 - DNA, 19 bp, synthetic construct
  source 1..19: mol_type = other DNA
tgcactaggcactgcagat

SEQ ID NO: 426 - DNA, 19 bp, synthetic construct
  source 1..19: mol_type = other DNA
tgcactagcaagatcagat

SEQ ID NO: 427 - DNA, 19 bp, synthetic construct
  source 1..19: mol_type = other DNA
tgcactagcagccagcgat

SEQ ID NO: 428 - DNA, 19 bp, synthetic construct
  source 1..19: mol_type = other DNA
tgcactagcatctggtgat

SEQ ID NO: 429 - DNA, 19 bp, synthetic construct
  source 1..19: mol_type = other DNA
tgcacttagcatcactgat

SEQ ID NO: 430 - DNA, 19 bp, synthetic construct
  source 1..19: mol_type = other DNA
tgcactaggcatgagtgat

SEQ ID NO: 431 - DNA, 21 bp, synthetic construct
  source 1..21: mol_type = other DNA
tgcactagcaatgcatatgat

SEQ ID NO: 432 - DNA, 19 bp, synthetic construct
  source 1..19: mol_type = other DNA
tgcactatagcaatgcgat

SEQ ID NO: 433 - DNA, 19 bp, synthetic construct
  source 1..19: mol_type = other DNA
tgcactatatagcaatgat

SEQ ID NO: 434 - DNA, 19 bp, synthetic construct
  source 1..19: mol_type = other DNA
tgcacttatatctcatgat

SEQ ID NO: 435 - DNA, 19 bp, synthetic construct
  source 1..19: mol_type = other DNA
tgcactattatgatatgat

SEQ ID NO: 436 - DNA, 19 bp, synthetic construct
  source 1..19: mol_type = other DNA
tgcactatattgcgatgat

SEQ ID NO: 437 - DNA, 19 bp, synthetic construct
  source 1..19: mol_type = other DNA
tgcactatcgcttgatgat

SEQ ID NO: 438 - DNA, 19 bp, synthetic construct
  source 1..19: mol_type = other DNA
tgcactatctcataatgat

SEQ ID NO: 439 - DNA, 19 bp, synthetic construct
  source 1..19: mol_type = other DNA
tgcacttatctgatctgat

SEQ ID NO: 440 - DNA, 21 bp, synthetic construct
  source 1..21: mol_type = other DNA
tgcactattcagatgcatgat

SEQ ID NO: 441 - DNA, 19 bp, synthetic construct
  source 1..19: mol_type = other DNA
tgcactatcaagctcagat

SEQ ID NO: 442 - DNA, 19 bp, synthetic construct
  source 1..19: mol_type = other DNA
tgcactatcatccagtgat

SEQ ID NO: 443 - DNA, 19 bp, synthetic construct
  source 1..19: mol_type = other DNA
tgcactatcatgtggtgat

SEQ ID NO: 444 - DNA, 19 bp, synthetic construct
  source 1..19: mol_type = other DNA
tgcacttatcatgcgcgat

SEQ ID NO: 445 - DNA, 19 bp, synthetic construct
  source 1..19: mol_type = other DNA
tgcactattgtatgctgat

SEQ ID NO: 446 - DNA, 19 bp, synthetic construct
  source 1..19: mol_type = other DNA
tgcactatgttgtgcagat

SEQ ID NO: 447 - DNA, 19 bp, synthetic construct
  source 1..19: mol_type = other DNA
tgcactatgtgccagagat

SEQ ID NO: 448 - DNA, 19 bp, synthetic construct
  source 1..19: mol_type = other DNA
tgcactatgtgcattcgat

SEQ ID NO: 449 - DNA, 19 bp, synthetic construct
  source 1..19: mol_type = other DNA
tgcacttatgagcgctgat

SEQ ID NO: 450 - DNA, 19 bp, synthetic construct
  source 1..19: mol_type = other DNA
tgcactattgatatgagat

SEQ ID NO: 451 - DNA, 19 bp, synthetic construct
  source 1..19: mol_type = other DNA
tgcactatgaatgagcgat

SEQ ID NO: 452 - DNA, 19 bp, synthetic construct
  source 1..19: mol_type = other DNA
tgcactatgcgaacatgat

SEQ ID NO: 453 - DNA, 19 bp, synthetic construct
  source 1..19: mol_type = other DNA
tgcactatgcgatggtgat

SEQ ID NO: 454 - DNA, 19 bp, synthetic construct
  source 1..19: mol_type = other DNA
tgcacttatgctatcagat

SEQ ID NO: 455 - DNA, 21 bp, synthetic construct
  source 1..21: mol_type = other DNA
tgcactattgctctgcatgat

SEQ ID NO: 456 - DNA, 21 bp, synthetic construct
  source 1..21: mol_type = other DNA
tgcactatgccacagcatgat

SEQ ID NO: 457 - DNA, 19 bp, synthetic construct
  source 1..19: mol_type = other DNA
tgcactatgcaccatcgat

SEQ ID NO: 458 - DNA, 19 bp, synthetic construct
  source 1..19: mol_type = other DNA
tgcactatgcagcggagat

SEQ ID NO: 459 - DNA, 19 bp, synthetic construct
  source 1..19: mol_type = other DNA
tgcacttatgcatgtagat

SEQ ID NO: 460 - DNA, 21 bp, synthetic construct
  source 1..21: mol_type = other DNA
tgcactattgcatgctctgat

SEQ ID NO: 461 - DNA, 21 bp, synthetic construct
  source 1..21: mol_type = other DNA
tgcactcgtggcatgcatgat

SEQ ID NO: 462 - DNA, 19 bp, synthetic construct
  source 1..19: mol_type = other DNA
tgcactcgagattgatgat

SEQ ID NO: 463 - DNA, 19 bp, synthetic construct
  source 1..19: mol_type = other DNA
tgcactcgatgagcctgat

SEQ ID NO: 464 - DNA, 19 bp, synthetic construct
  source 1..19: mol_type = other DNA
tgcacttcgatgctgtgat

SEQ ID NO: 465 - DNA, 19 bp, synthetic construct
  source 1..19: mol_type = other DNA
tgcactcggcgcgcatgat

SEQ ID NO: 466 - DNA, 19 bp, synthetic construct
  source 1..19: mol_type = other DNA
tgcactcgcggcatatgat

SEQ ID NO: 467 - DNA, 19 bp, synthetic construct
  source 1..19: mol_type = other DNA
tgcactcgcgcaatgcgat

SEQ ID NO: 468 - DNA, 19 bp, synthetic construct
  source 1..19: mol_type = other DNA
tgcactcgctgcaggtgat

SEQ ID NO: 469 - DNA, 19 bp, synthetic construct
  source 1..19: mol_type = other DNA
tgcacttcgcacacatgat

SEQ ID NO: 470 - DNA, 19 bp, synthetic construct
  source 1..19: mol_type = other DNA
tgcactcggcacatgtgat

SEQ ID NO: 471 - DNA, 19 bp, synthetic construct
  source 1..19: mol_type = other DNA
tgcactcgcaatagatgat

SEQ ID NO: 472 - DNA, 21 bp, synthetic construct
  source 1..21: mol_type = other DNA
tgcactcgcatccatgcagat

SEQ ID NO: 473 - DNA, 19 bp, synthetic construct
  source 1..19: mol_type = other DNA
tgcactcgcatgtggagat

SEQ ID NO: 474 - DNA, 21 bp, synthetic construct
  source 1..21: mol_type = other DNA
tgcactcgcatgatcaatgat

SEQ ID NO: 475 - DNA, 19 bp, synthetic construct
  source 1..19: mol_type = other DNA
tgcacttcgcatgctcgat

SEQ ID NO: 476 - DNA, 19 bp, synthetic construct
  source 1..19: mol_type = other DNA
tgcactcttagcatgtgat

SEQ ID NO: 477 - DNA, 19 bp, synthetic construct
  source 1..19: mol_type = other DNA
tgcactctattcatatgat

SEQ ID NO: 478 - DNA, 19 bp, synthetic construct
  source 1..19: mol_type = other DNA
tgcactctatgttgctgat

SEQ ID NO: 479 - DNA, 19 bp, synthetic construct
  source 1..19: mol_type = other DNA
tgcactctatgcgccagat

SEQ ID NO: 480 - DNA, 21 bp, synthetic construct
  source 1..21: mol_type = other DNA
tgcactctcgcatgcaatgat

SEQ ID NO: 481 - DNA, 19 bp, synthetic construct
  source 1..19: mol_type = other DNA
tgcacttctctatgatgat

SEQ ID NO: 482 - DNA, 19 bp, synthetic construct
  source 1..19: mol_type = other DNA
tgcactcttctcagcagat

SEQ ID NO: 483 - DNA, 19 bp, synthetic construct
  source 1..19: mol_type = other DNA
tgcactctcttgatgcgat

SEQ ID NO: 484 - DNA, 19 bp, synthetic construct
  source 1..19: mol_type = other DNA
tgcactctcacaagctgat

SEQ ID NO: 485 - DNA, 19 bp, synthetic construct
  source 1..19: mol_type = other DNA
tgcactctcacatggagat

SEQ ID NO: 486 - DNA, 21 bp, synthetic construct
  source 1..21: mol_type = other DNA
tgcactctcatctgcaatgat

SEQ ID NO: 487 - DNA, 19 bp, synthetic construct
  source 1..19: mol_type = other DNA
tgcacttctcatgtcagat

SEQ ID NO: 488 - DNA, 21 bp, synthetic construct
  source 1..21: mol_type = other DNA
tgcactcttcatgagcatgat

SEQ ID NO: 489 - DNA, 19 bp, synthetic construct
  source 1..19: mol_type = other DNA
tgcactctcaatgcacgat

SEQ ID NO: 490 - DNA, 19 bp, synthetic construct
  source 1..19: mol_type = other DNA
tgcactctgtattgcagat

SEQ ID NO: 491 - DNA, 19 bp, synthetic construct
  source 1..19: mol_type = other DNA
tgcactctgtcagcctgat

SEQ ID NO: 492 - DNA, 19 bp, synthetic construct
  source 1..19: mol_type = other DNA
tgcacttctgtgagatgat

SEQ ID NO: 493 - DNA, 19 bp, synthetic construct
  source 1..19: mol_type = other DNA
tgcactcttgtgatctgat

SEQ ID NO: 494 - DNA, 19 bp, synthetic construct
  source 1..19: mol_type = other DNA
tgcactctgttgctcagat

SEQ ID NO: 495 - DNA, 21 bp, synthetic construct
  source 1..21: mol_type = other DNA
tgcactctgacaatgcatgat

SEQ ID NO: 496 - DNA, 19 bp, synthetic construct
  source 1..19: mol_type = other DNA
tgcactctgagtgccagat

SEQ ID NO: 497 - DNA, 19 bp, synthetic construct
  source 1..19: mol_type = other DNA
tgcacttctgatgatagat

SEQ ID NO: 498 - DNA, 21 bp, synthetic construct
  source 1..21: mol_type = other DNA
tgcactcttgatgcacatgat

SEQ ID NO: 499 - DNA, 21 bp, synthetic construct
  source 1..21: mol_type = other DNA
tgcactctgaatgcatgcgat

SEQ ID NO: 500 - DNA, 19 bp, synthetic construct
  source 1..19: mol_type = other DNA
tgcactctgctcctgcgat

SEQ ID NO: 501 - DNA, 21 bp, synthetic construct
  source 1..21: mol_type = other DNA
tgcactctgctcattcatgat

SEQ ID NO: 502 - DNA, 21 bp, synthetic construct
  source 1..21: mol_type = other DNA
tgcactctgctgtgcaatgat

SEQ ID NO: 503 - DNA, 21 bp, synthetic construct
  source 1..21: mol_type = other DNA
tgcacttctgctgcatgagat

SEQ ID NO: 504 - DNA, 19 bp, synthetic construct
  source 1..19: mol_type = other DNA
tgcactcttgcagagcgat

SEQ ID NO: 505 - DNA, 19 bp, synthetic construct
  source 1..19: mol_type = other DNA
tgcactctgccagcgtgat

SEQ ID NO: 506 - DNA, 21 bp, synthetic construct
  source 1..21: mol_type = other DNA
tgcactctgcaggctcatgat

SEQ ID NO: 507 - DNA, 21 bp, synthetic construct
  source 1..21: mol_type = other DNA
tgcactctgcatattgctgat

SEQ ID NO: 508 - DNA, 19 bp, synthetic construct
  source 1..19: mol_type = other DNA
tgcacttcactcatgagat

SEQ ID NO: 509 - DNA, 19 bp, synthetic construct
  source 1..19: mol_type = other DNA
tgcactcaactgatatgat

SEQ ID NO: 510 - DNA, 19 bp, synthetic construct
  source 1..19: mol_type = other DNA
tgcactcacttgctgtgat

SEQ ID NO: 511 - DNA, 19 bp, synthetic construct
  source 1..19: mol_type = other DNA
tgcactcacagttgatgat

SEQ ID NO: 512 - DNA, 19 bp, synthetic construct
  source 1..19: mol_type = other DNA
tgcactcacagacaatgat

SEQ ID NO: 513 - DNA, 19 bp, synthetic construct
  source 1..19: mol_type = other DNA
tgcacttcacagcatagat

SEQ ID NO: 514 - DNA, 19 bp, synthetic construct
  source 1..19: mol_type = other DNA
tgcactcaacatatctgat

SEQ ID NO: 515 - DNA, 19 bp, synthetic construct
  source 1..19: mol_type = other DNA
tgcactcacaatcgcagat

SEQ ID NO: 516 - DNA, 19 bp, synthetic construct
  source 1..19: mol_type = other DNA
tgcactcacatggagagat

SEQ ID NO: 517 - DNA, 21 bp, synthetic construct
  source 1..21: mol_type = other DNA
tgcactcacatgcaatgcgat

SEQ ID NO: 518 - DNA, 19 bp, synthetic construct
  source 1..19: mol_type = other DNA
tgcacttcagtgagcagat

SEQ ID NO: 519 - DNA, 19 bp, synthetic construct
  source 1..19: mol_type = other DNA
tgcactcaagagcagtgat

SEQ ID NO: 520 - DNA, 19 bp, synthetic construct
  source 1..19: mol_type = other DNA
tgcactcagaagcatcgat

SEQ ID NO: 521 - DNA, 21 bp, synthetic construct
  source 1..21: mol_type = other DNA
tgcactcagatcctgcatgat

SEQ ID NO: 522 - DNA, 19 bp, synthetic construct
  source 1..19: mol_type = other DNA
tgcactcagatgtccagat

SEQ ID NO: 523 - DNA, 21 bp, synthetic construct
  source 1..21: mol_type = other DNA
tgcactcagcgatgcaatgat

SEQ ID NO: 524 - DNA, 19 bp, synthetic construct
  source 1..19: mol_type = other DNA
tgcacttcagcgctctgat

SEQ ID NO: 525 - DNA, 19 bp, synthetic construct
  source 1..19: mol_type = other DNA
tgcactcaagcgcacagat

SEQ ID NO: 526 - DNA, 21 bp, synthetic construct
  source 1..21: mol_type = other DNA
tgcactcagcctcatgctgat

SEQ ID NO: 527 - DNA, 19 bp, synthetic construct
  source 1..19: mol_type = other DNA
tgcactcagctggatcgat

SEQ ID NO: 528 - DNA, 19 bp, synthetic construct
  source 1..19: mol_type = other DNA
tgcactcagctgcggcgat

SEQ ID NO: 529 - DNA, 19 bp, synthetic construct
  source 1..19: mol_type = other DNA
tgcacttcagcatacagat

SEQ ID NO: 530 - DNA, 21 bp, synthetic construct
  source 1..21: mol_type = other DNA
tgcactcaagcatgtgctgat

SEQ ID NO: 531 - DNA, 21 bp, synthetic construct
  source 1..21: mol_type = other DNA
tgcactcagccatgcgatgat

SEQ ID NO: 532 - DNA, 21 bp, synthetic construct
  source 1..21: mol_type = other DNA
tgcactcatagcctgcatgat

SEQ ID NO: 533 - DNA, 19 bp, synthetic construct
  source 1..19: mol_type = other DNA
tgcactcatatcgcctgat

SEQ ID NO: 534 - DNA, 19 bp, synthetic construct
  source 1..19: mol_type = other DNA
tgcacttcatatctgagat

SEQ ID NO: 535 - DNA, 21 bp, synthetic construct
  source 1..21: mol_type = other DNA
tgcactcaatatgatgcagat

SEQ ID NO: 536 - DNA, 21 bp, synthetic construct
  source 1..21: mol_type = other DNA
tgcactcataatgcatctgat

SEQ ID NO: 537 - DNA, 19 bp, synthetic construct
  source 1..19: mol_type = other DNA
tgcactcatcgccactgat

SEQ ID NO: 538 - DNA, 19 bp, synthetic construct
  source 1..19: mol_type = other DNA
tgcactcatcgcaggagat

SEQ ID NO: 539 - DNA, 21 bp, synthetic construct
  source 1..21: mol_type = other DNA
tgcactcatctgcgcaatgat

SEQ ID NO: 540 - DNA, 21 bp, synthetic construct
  source 1..21: mol_type = other DNA
tgcacttcatctgcatcagat

SEQ ID NO: 541 - DNA, 21 bp, synthetic construct
  source 1..21: mol_type = other DNA
tgcactcaatcacatcatgat

SEQ ID NO: 542 - DNA, 19 bp, synthetic construct
  source 1..19: mol_type = other DNA
tgcactcatggtatatgat

SEQ ID NO: 543 - DNA, 19 bp, synthetic construct
  source 1..19: mol_type = other DNA
tgcactcatgtccacagat

SEQ ID NO: 544 - DNA, 19 bp, synthetic construct
  source 1..19: mol_type = other DNA
tgcactcatgtgtggtgat

SEQ ID NO: 545 - DNA, 19 bp, synthetic construct
  source 1..19: mol_type = other DNA
tgcacttcatgacagagat

SEQ ID NO: 546 - DNA, 21 bp, synthetic construct
  source 1..21: mol_type = other DNA
tgcactcaatgagagcatgat

SEQ ID NO: 547 - DNA, 21 bp, synthetic construct
  source 1..21: mol_type = other DNA
tgcactcatggagcatatgat

SEQ ID NO: 548 - DNA, 19 bp, synthetic construct
  source 1..19: mol_type = other DNA
tgcactcatgattactgat

SEQ ID NO: 549 - DNA, 21 bp, synthetic construct
  source 1..21: mol_type = other DNA
tgcactcatgatcaatgtgat

SEQ ID NO: 550 - DNA, 19 bp, synthetic construct
  source 1..19: mol_type = other DNA
tgcacttcatgcgtatgat

SEQ ID NO: 551 - DNA, 21 bp, synthetic construct
  source 1..21: mol_type = other DNA
tgcactcaatgcgctgcagat

SEQ ID NO: 552 - DNA, 21 bp, synthetic construct
  source 1..21: mol_type = other DNA
tgcactcatggctagcatgat

SEQ ID NO: 553 - DNA, 21 bp, synthetic construct
  source 1..21: mol_type = other DNA
tgcactcatgcaactgatgat

SEQ ID NO: 554 - DNA, 21 bp, synthetic construct
  source 1..21: mol_type = other DNA
tgcactcatgcagaatctgat

SEQ ID NO: 555 - DNA, 21 bp, synthetic construct
  source 1..21: mol_type = other DNA
tgcactcatgcagatggagat

SEQ ID NO: 556 - DNA, 21 bp, synthetic construct
  source 1..21: mol_type = other DNA
tgcacttcatgcatctcagat

SEQ ID NO: 557 - DNA, 21 bp, synthetic construct
  source 1..21: mol_type = other DNA
tgcactcaatgcatcagcgat

SEQ ID NO: 558 - DNA, 19 bp, synthetic construct
  source 1..19: mol_type = other DNA
tgcactgtaggcatctgat

SEQ ID NO: 559 - DNA, 19 bp, synthetic construct
  source 1..19: mol_type = other DNA
tgcactgtagcaatgagat

SEQ ID NO: 560 - DNA, 19 bp, synthetic construct
  source 1..19: mol_type = other DNA
tgcactgtatgtgccagat

SEQ ID NO: 561 - DNA, 19 bp, synthetic construct
  source 1..19: mol_type = other DNA
tgcacttgtatgatgtgat

SEQ ID NO: 562 - DNA, 19 bp, synthetic construct
  source 1..19: mol_type = other DNA
tgcactgttcgctgcagat

SEQ ID NO: 563 - DNA, 19 bp, synthetic construct
  source 1..19: mol_type = other DNA
tgcactgtcggcagctgat

SEQ ID NO: 564 - DNA, 19 bp, synthetic construct
  source 1..19: mol_type = other DNA
tgcactgtctgaatatgat

SEQ ID NO: 565 - DNA, 19 bp, synthetic construct
  source 1..19: mol_type = other DNA
tgcactgtctgctggtgat

SEQ ID NO: 566 - DNA, 19 bp, synthetic construct
  source 1..19: mol_type = other DNA
tgcacttgtcacgcatgat

SEQ ID NO: 567 - DNA, 19 bp, synthetic construct
  source 1..19: mol_type = other DNA
tgcactgttcacatcagat

SEQ ID NO: 568 - DNA, 19 bp, synthetic construct
  source 1..19: mol_type = other DNA
tgcactgtcaagagcagat

SEQ ID NO: 569 - DNA, 19 bp, synthetic construct
  source 1..19: mol_type = other DNA
tgcactgtcagcctatgat

SEQ ID NO: 570 - DNA, 19 bp, synthetic construct
  source 1..19: mol_type = other DNA
tgcactgtcatcacctgat

SEQ ID NO: 571 - DNA, 19 bp, synthetic construct
  source 1..19: mol_type = other DNA
tgcacttgtcatgtctgat

SEQ ID NO: 572 - DNA, 21 bp, synthetic construct
  source 1..21: mol_type = other DNA
tgcactgttcatgcagatgat

SEQ ID NO: 573 - DNA, 21 bp, synthetic construct
  source 1..21: mol_type = other DNA
tgcactgtcaatgcatgcgat

SEQ ID NO: 574 - DNA, 19 bp, synthetic construct
  source 1..19: mol_type = other DNA
tgcactgtgtaggcatgat

SEQ ID NO: 575 - DNA, 19 bp, synthetic construct
  source 1..19: mol_type = other DNA
tgcactgtgtcatcctgat

SEQ ID NO: 576 - DNA, 19 bp, synthetic construct
  source 1..19: mol_type = other DNA
tgcacttgtgtcatgagat

SEQ ID NO: 577 - DNA, 19 bp, synthetic construct
  source 1..19: mol_type = other DNA
tgcactgttgtgatcagat

SEQ ID NO: 578 - DNA, 19 bp, synthetic construct
  source 1..19: mol_type = other DNA
tgcactgtgttgcgatgat

SEQ ID NO: 579 - DNA, 19 bp, synthetic construct
  source 1..19: mol_type = other DNA
tgcactgtgacaagctgat

SEQ ID NO: 580 - DNA, 19 bp, synthetic construct
  source 1..19: mol_type = other DNA
tgcactgtgacataatgat

SEQ ID NO: 581 - DNA, 19 bp, synthetic construct
  source 1..19: mol_type = other DNA
tgcacttgtgagtgctgat

SEQ ID NO: 582 - DNA, 19 bp, synthetic construct
  source 1..19: mol_type = other DNA
tgcactgttgagctcagat

SEQ ID NO: 583 - DNA, 19 bp, synthetic construct
  source 1..19: mol_type = other DNA
tgcactgtgaatatgcgat

SEQ ID NO: 584 - DNA, 19 bp, synthetic construct
  source 1..19: mol_type = other DNA
tgcactgtgatccgcagat

SEQ ID NO: 585 - DNA, 19 bp, synthetic construct
  source 1..19: mol_type = other DNA
tgcactgtgatgtaatgat

SEQ ID NO: 586 - DNA, 19 bp, synthetic construct
  source 1..19: mol_type = other DNA
tgcacttgtgatgactgat

SEQ ID NO: 587 - DNA, 19 bp, synthetic construct
  source 1..19: mol_type = other DNA
tgcactgttgcgtgatgat

SEQ ID NO: 588 - DNA, 19 bp, synthetic construct
  source 1..19: mol_type = other DNA
tgcactgtgccgatctgat

SEQ ID NO: 589 - DNA, 19 bp, synthetic construct
  source 1..19: mol_type = other DNA
tgcactgtgcgccatagat

SEQ ID NO: 590 - DNA, 19 bp, synthetic construct
  source 1..19: mol_type = other DNA
tgcactgtgctcaccagat

SEQ ID NO: 591 - DNA, 19 bp, synthetic construct
  source 1..19: mol_type = other DNA
tgcacttgtgctcagtgat

SEQ ID NO: 592 - DNA, 19 bp, synthetic construct
  source 1..19: mol_type = other DNA
tgcactgttgctgtgcgat

SEQ ID NO: 593 - DNA, 19 bp, synthetic construct
  source 1..19: mol_type = other DNA
tgcactgtgcctgagagat

SEQ ID NO: 594 - DNA, 19 bp, synthetic construct
  source 1..19: mol_type = other DNA
tgcactgtgcaggagtgat

SEQ ID NO: 595 - DNA, 19 bp, synthetic construct
  source 1..19: mol_type = other DNA
tgcactgtgcatcttagat

SEQ ID NO: 596 - DNA, 21 bp, synthetic construct
  source 1..21: mol_type = other DNA
tgcactgtgcatctgcctgat

SEQ ID NO: 597 - DNA, 21 bp, synthetic construct
  source 1..21: mol_type = other DNA
tgcacttgactgctgcatgat

SEQ ID NO: 598 - DNA, 19 bp, synthetic construct
  source 1..19: mol_type = other DNA
tgcactgaacacatgcgat

SEQ ID NO: 599 - DNA, 19 bp, synthetic construct
  source 1..19: mol_type = other DNA
tgcactgacaagatctgat

SEQ ID NO: 600 - DNA, 19 bp, synthetic construct
  source 1..19: mol_type = other DNA
tgcactgagtgaagctgat

SEQ ID NO: 601 - DNA, 19 bp, synthetic construct
  source 1..19: mol_type = other DNA
tgcactgagtgatggagat

SEQ ID NO: 602 - DNA, 19 bp, synthetic construct
  source 1..19: mol_type = other DNA
tgcacttgagacatgagat

SEQ ID NO: 603 - DNA, 21 bp, synthetic construct
  source 1..21: mol_type = other DNA
tgcactgaagatcagcatgat

SEQ ID NO: 604 - DNA, 19 bp, synthetic construct
  source 1..19: mol_type = other DNA
tgcactgagaatgtgtgat

SEQ ID NO: 605 - DNA, 19 bp, synthetic construct
  source 1..19: mol_type = other DNA
tgcactgagatggatcgat

SEQ ID NO: 606 - DNA, 19 bp, synthetic construct
  source 1..19: mol_type = other DNA
tgcactgagcgctggcgat

SEQ ID NO: 607 - DNA, 19 bp, synthetic construct
  source 1..19: mol_type = other DNA
tgcacttgagcgcactgat

SEQ ID NO: 608 - DNA, 19 bp, synthetic construct
  source 1..19: mol_type = other DNA
tgcactgaagctatatgat

SEQ ID NO: 609 - DNA, 19 bp, synthetic construct
  source 1..19: mol_type = other DNA
tgcactgagcctgtcagat

SEQ ID NO: 610 - DNA, 21 bp, synthetic construct
  source 1..21: mol_type = other DNA
tgcactgagcaggtgcatgat

SEQ ID NO: 611 - DNA, 21 bp, synthetic construct
  source 1..21: mol_type = other DNA
tgcactgagcagcaagatgat

SEQ ID NO: 612 - DNA, 19 bp, synthetic construct
  source 1..19: mol_type = other DNA
tgcacttgagcatagagat

SEQ ID NO: 613 - DNA, 21 bp, synthetic construct
  source 1..21: mol_type = other DNA
tgcactgaagcatatgctgat

SEQ ID NO: 614 - DNA, 21 bp, synthetic construct
  source 1..21: mol_type = other DNA
tgcactgagccatcatcagat

SEQ ID NO: 615 - DNA, 21 bp, synthetic construct
  source 1..21: mol_type = other DNA
tgcactgagcattgcgctgat

SEQ ID NO: 616 - DNA, 19 bp, synthetic construct
  source 1..19: mol_type = other DNA
tgcactgatacagaatgat

SEQ ID NO: 617 - DNA, 19 bp, synthetic construct
  source 1..19: mol_type = other DNA
tgcacttgatatcagcgat

SEQ ID NO: 618 - DNA, 21 bp, synthetic construct
  source 1..21: mol_type = other DNA
tgcactgaatatgctgctgat

SEQ ID NO: 619 - DNA, 21 bp, synthetic construct
  source 1..21: mol_type = other DNA
tgcactgataatgcacatgat

SEQ ID NO: 620 - DNA, 19 bp, synthetic construct
  source 1..19: mol_type = other DNA
tgcactgatcgaatcagat

SEQ ID NO: 621 - DNA, 19 bp, synthetic construct
  source 1..19: mol_type = other DNA
tgcactgatcgctcctgat

SEQ ID NO: 622 - DNA, 21 bp, synthetic construct
  source 1..21: mol_type = other DNA
tgcactgatctatgcaatgat

SEQ ID NO: 623 - DNA, 19 bp, synthetic construct
  source 1..19: mol_type = other DNA
tgcacttgatctcgctgat

SEQ ID NO: 624 - DNA, 19 bp, synthetic construct
  source 1..19: mol_type = other DNA
tgcactgaatctgtgagat

SEQ ID NO: 625 - DNA, 19 bp, synthetic construct
  source 1..19: mol_type = other DNA
tgcactgatcctgcacgat

SEQ ID NO: 626 - DNA, 19 bp, synthetic construct
  source 1..19: mol_type = other DNA
tgcactgatcacctgagat

SEQ ID NO: 627 - DNA, 19 bp, synthetic construct
  source 1..19: mol_type = other DNA
tgcactgatcagtggcgat

SEQ ID NO: 628 - DNA, 19 bp, synthetic construct
  source 1..19: mol_type = other DNA
tgcacttgatcatacagat

SEQ ID NO: 629 - DNA, 21 bp, synthetic construct
  source 1..21: mol_type = other DNA
tgcactgaatcatgcatagat

SEQ ID NO: 630 - DNA, 21 bp, synthetic construct
  source 1..21: mol_type = other DNA
tgcactgatggtgctcatgat

SEQ ID NO: 631 - DNA, 21 bp, synthetic construct
  source 1..21: mol_type = other DNA
tgcactgatgaggatcatgat

SEQ ID NO: 632 - DNA, 21 bp, synthetic construct
  source 1..21: mol_type = other DNA
tgcactgatgagcttgatgat

SEQ ID NO: 633 - DNA, 21 bp, synthetic construct
  source 1..21: mol_type = other DNA
tgcactgatgagcagccagat

SEQ ID NO: 634 - DNA, 19 bp, synthetic construct
  source 1..19: mol_type = other DNA
tgcacttgatgatagtgat

SEQ ID NO: 635 - DNA, 19 bp, synthetic construct
  source 1..19: mol_type = other DNA
tgcactgaatgatctcgat

SEQ ID NO: 636 - DNA, 21 bp, synthetic construct
  source 1..21: mol_type = other DNA
tgcactgatggcgcgcatgat

SEQ ID NO: 637 - DNA, 19 bp, synthetic construct
  source 1..19: mol_type = other DNA
tgcactgatgcttagcgat

SEQ ID NO: 638 - DNA, 21 bp, synthetic construct
  source 1..21: mol_type = other DNA
tgcactgatgcatccgatgat

SEQ ID NO: 639 - DNA, 19 bp, synthetic construct
  source 1..19: mol_type = other DNA
tgcacttgcgtgcatagat

SEQ ID NO: 640 - DNA, 21 bp, synthetic construct
  source 1..21: mol_type = other DNA
tgcactgccgagcagcatgat

SEQ ID NO: 641 - DNA, 19 bp, synthetic construct
  source 1..19: mol_type = other DNA
tgcactgcgaatatatgat

SEQ ID NO: 642 - DNA, 19 bp, synthetic construct
  source 1..19: mol_type = other DNA
tgcactgcgatccacagat

SEQ ID NO: 643 - DNA, 21 bp, synthetic construct
  source 1..21: mol_type = other DNA
tgcactgcgatgtggcatgat

SEQ ID NO: 644 - DNA, 21 bp, synthetic construct
  source 1..21: mol_type = other DNA
tgcactgcgctatgcaatgat

SEQ ID NO: 645 - DNA, 19 bp, synthetic construct
  source 1..19: mol_type = other DNA
tgcacttgcgctctcagat

SEQ ID NO: 646 - DNA, 21 bp, synthetic construct
  source 1..21: mol_type = other DNA
tgcactgccgctgctgatgat

SEQ ID NO: 647 - DNA, 21 bp, synthetic construct
  source 1..21: mol_type = other DNA
tgcactgcgcctgcacatgat

SEQ ID NO: 648 - DNA, 19 bp, synthetic construct
  source 1..19: mol_type = other DNA
tgcactgcgcaggatagat

SEQ ID NO: 649 - DNA, 21 bp, synthetic construct
  source 1..21: mol_type = other DNA
tgcactgcgcagcttgcagat

SEQ ID NO: 650 - DNA, 21 bp, synthetic construct
  source 1..21: mol_type = other DNA
tgcactgcgcagcatcctgat

SEQ ID NO: 651 - DNA, 21 bp, synthetic construct
  source 1..21: mol_type = other DNA
tgcacttgcgcatcagctgat

SEQ ID NO: 652 - DNA, 21 bp, synthetic construct
  source 1..21: mol_type = other DNA
tgcactgccgcatgagcagat

SEQ ID NO: 653 - DNA, 21 bp, synthetic construct
  source 1..21: mol_type = other DNA
tgcactgcgccatgatgtgat

SEQ ID NO: 654 - DNA, 19 bp, synthetic construct
  source 1..19: mol_type = other DNA
tgcactgctacaagcagat

SEQ ID NO: 655 - DNA, 19 bp, synthetic construct
  source 1..19: mol_type = other DNA
tgcactgctatctggtgat

SEQ ID NO: 656 - DNA, 19 bp, synthetic construct
  source 1..19: mol_type = other DNA
tgcacttgctatgagcgat

SEQ ID NO: 657 - DNA, 19 bp, synthetic construct
  source 1..19: mol_type = other DNA
tgcactgcctatgctagat

SEQ ID NO: 658 - DNA, 19 bp, synthetic construct
  source 1..19: mol_type = other DNA
tgcactgctccgcatcgat

SEQ ID NO: 659 - DNA, 21 bp, synthetic construct
  source 1..21: mol_type = other DNA
tgcactgctctccatgctgat

SEQ ID NO: 660 - DNA, 21 bp, synthetic construct
  source 1..21: mol_type = other DNA
tgcactgctctgcttcatgat

SEQ ID NO: 661 - DNA, 19 bp, synthetic construct
  source 1..19: mol_type = other DNA
tgcacttgctcagtgtgat

SEQ ID NO: 662 - DNA, 21 bp, synthetic construct
  source 1..21: mol_type = other DNA
tgcactgcctcagatcatgat

SEQ ID NO: 663 - DNA, 19 bp, synthetic construct
  source 1..19: mol_type = other DNA
tgcactgctccagcgagat

SEQ ID NO: 664 - DNA, 21 bp, synthetic construct
  source 1..21: mol_type = other DNA
tgcactgctcattgatgagat

SEQ ID NO: 665 - DNA, 21 bp, synthetic construct
  source 1..21: mol_type = other DNA
tgcactgctgtctggcatgat

SEQ ID NO: 666 - DNA, 19 bp, synthetic construct
  source 1..19: mol_type = other DNA
tgcacttgctgtgcgcgat

SEQ ID NO: 667 - DNA, 21 bp, synthetic construct
  source 1..21: mol_type = other DNA
tgcactgcctgatcagatgat

SEQ ID NO: 668 - DNA, 19 bp, synthetic construct
  source 1..19: mol_type = other DNA
tgcactgctggatgtcgat

SEQ ID NO: 669 - DNA, 21 bp, synthetic construct
  source 1..21: mol_type = other DNA
tgcactgctgcggagcatgat

SEQ ID NO: 670 - DNA, 19 bp, synthetic construct
  source 1..19: mol_type = other DNA
tgcactgctgctgaacgat

SEQ ID NO: 671 - DNA, 21 bp, synthetic construct
  source 1..21: mol_type = other DNA
tgcactgctgcatcattcgat

SEQ ID NO: 672 - DNA, 19 bp, synthetic construct
  source 1..19: mol_type = other DNA
tgcacttgcacgatgagat

SEQ ID NO: 673 - DNA, 19 bp, synthetic construct
  source 1..19: mol_type = other DNA
tgcactgccacgcgatgat

SEQ ID NO: 674 - DNA, 19 bp, synthetic construct
  source 1..19: mol_type = other DNA
tgcactgcaccgctcagat

SEQ ID NO: 675 - DNA, 19 bp, synthetic construct
  source 1..19: mol_type = other DNA
tgcactgcactaagcagat

SEQ ID NO: 676 - DNA, 19 bp, synthetic construct
  source 1..19: mol_type = other DNA
tgcactgcactcacctgat

SEQ ID NO: 677 - DNA, 21 bp, synthetic construct
  source 1..21: mol_type = other DNA
tgcactgcacactgcaatgat

SEQ ID NO: 678 - DNA, 19 bp, synthetic construct
  source 1..19: mol_type = other DNA
tgcacttgcacagagcgat

SEQ ID NO: 679 - DNA, 19 bp, synthetic construct
  source 1..19: mol_type = other DNA
tgcactgccacatcgtgat

SEQ ID NO: 680 - DNA, 21 bp, synthetic construct
  source 1..21: mol_type = other DNA
tgcactgcaccatcatatgat

SEQ ID NO: 681 - DNA, 19 bp, synthetic construct
  source 1..19: mol_type = other DNA
tgcactgcacattgtagat

SEQ ID NO: 682 - DNA, 21 bp, synthetic construct
  source 1..21: mol_type = other DNA
tgcactgcagtgattcatgat

SEQ ID NO: 683 - DNA, 21 bp, synthetic construct
  source 1..21: mol_type = other DNA
tgcactgcagtgctgcctgat

SEQ ID NO: 684 - DNA, 21 bp, synthetic construct
  source 1..21: mol_type = other DNA
tgcacttgcagtgcagatgat

SEQ ID NO: 685 - DNA, 19 bp, synthetic construct
  source 1..19: mol_type = other DNA
tgcactgccagactgtgat

SEQ ID NO: 686 - DNA, 21 bp, synthetic construct
  source 1..21: mol_type = other DNA
tgcactgcaggacatcatgat

SEQ ID NO: 687 - DNA, 21 bp, synthetic construct
  source 1..21: mol_type = other DNA
tgcactgcagaggatgctgat

SEQ ID NO: 688 - DNA, 21 bp, synthetic construct
  source 1..21: mol_type = other DNA
tgcactgcagatgcctatgat

SEQ ID NO: 689 - DNA, 19 bp, synthetic construct
  source 1..19: mol_type = other DNA
tgcacttgcagcgagtgat

SEQ ID NO: 690 - DNA, 21 bp, synthetic construct
  source 1..21: mol_type = other DNA
tgcactgccagcacagcagat

SEQ ID NO: 691 - DNA, 21 bp, synthetic construct
  source 1..21: mol_type = other DNA
tgcactgcaggcatctctgat

SEQ ID NO: 692 - DNA, 21 bp, synthetic construct
  source 1..21: mol_type = other DNA
tgcactgcagcaatgcacgat

SEQ ID NO: 693 - DNA, 19 bp, synthetic construct
  source 1..19: mol_type = other DNA
tgcactgcatagattagat

SEQ ID NO: 694 - DNA, 19 bp, synthetic construct
  source 1..19: mol_type = other DNA
tgcacttgcatagcgtgat

SEQ ID NO: 695 - DNA, 19 bp, synthetic construct
  source 1..19: mol_type = other DNA
tgcactgccatagcacgat

SEQ ID NO: 696 - DNA, 21 bp, synthetic construct
  source 1..21: mol_type = other DNA
tgcactgcattatatcatgat

SEQ ID NO: 697 - DNA, 21 bp, synthetic construct
  source 1..21: mol_type = other DNA
tgcactgcatattgtgatgat

SEQ ID NO: 698 - DNA, 21 bp, synthetic construct
  source 1..21: mol_type = other DNA
tgcactgcatcgtggcatgat

SEQ ID NO: 699 - DNA, 21 bp, synthetic construct
  source 1..21: mol_type = other DNA
tgcactgcatctctgaatgat

SEQ ID NO: 700 - DNA, 21 bp, synthetic construct
  source 1..21: mol_type = other DNA
tgcacttgcatctgacatgat

SEQ ID NO: 701 - DNA, 21 bp, synthetic construct
  source 1..21: mol_type = other DNA
tgcactgccatcatagatgat

SEQ ID NO: 702 - DNA, 21 bp, synthetic construct
  source 1..21: mol_type = other DNA
tgcactgcattcatctgcgat

SEQ ID NO: 703 - DNA, 21 bp, synthetic construct
  source 1..21: mol_type = other DNA
tgcactgcatgttgctgagat

SEQ ID NO: 704 - DNA, 21 bp, synthetic construct
  source 1..21: mol_type = other DNA
tgcactgcatgacaatgcgat

SEQ ID NO: 705 - DNA, 21 bp, synthetic construct
  source 1..21: mol_type = other DNA
tgcactgcatgatagccagat

SEQ ID NO: 706 - DNA, 21 bp, synthetic construct
  source 1..21: mol_type = other DNA
tgcacttgcatgcgatgcgat

SEQ ID NO: 707 - DNA, 21 bp, synthetic construct
  source 1..21: mol_type = other DNA
tgcactgccatgctatgagat

SEQ ID NO: 708 - DNA, 21 bp, synthetic construct
  source 1..21: mol_type = other DNA
tgcactgcattgctcgcagat

SEQ ID NO: 709 - DNA, 21 bp, synthetic construct
  source 1..21: mol_type = other DNA
tgcactgcatgcctgtctgat

SEQ ID NO: 710 - DNA, 21 bp, synthetic construct
  source 1..21: mol_type = other DNA
tgcactgcatgctggcgtgat

SEQ ID NO: 711 - DNA, 21 bp, synthetic construct
  source 1..21: mol_type = other DNA
tgcactgcatgcacacctgat

SEQ ID NO: 712 - DNA, 21 bp, synthetic construct
  source 1..21: mol_type = other DNA
tgcacttgcatgcagtcagat

SEQ ID NO: 713 - DNA, 21 bp, synthetic construct
  source 1..21: mol_type = other DNA
tgcactgccatgcagcgcgat

SEQ ID NO: 714 - DNA, 19 bp, synthetic construct
  source 1..19: mol_type = other DNA
tgcacacgtggcacatgat

SEQ ID NO: 715 - DNA, 19 bp, synthetic construct
  source 1..19: mol_type = other DNA
tgcacacgtgcaatgcgat

SEQ ID NO: 716 - DNA, 19 bp, synthetic construct
  source 1..19: mol_type = other DNA
tgcacacgagcgcaatgat

SEQ ID NO: 717 - DNA, 19 bp, synthetic construct
  source 1..19: mol_type = other DNA
tgcacaacgagcatatgat

SEQ ID NO: 718 - DNA, 19 bp, synthetic construct
  source 1..19: mol_type = other DNA
tgcacacggatagcatgat

SEQ ID NO: 719 - DNA, 19 bp, synthetic construct
  source 1..19: mol_type = other DNA
tgcacacgattctgatgat

SEQ ID NO: 720 - DNA, 19 bp, synthetic construct
  source 1..19: mol_type = other DNA
tgcacacgcgaggcatgat

SEQ ID NO: 721 - DNA, 19 bp, synthetic construct
  source 1..19: mol_type = other DNA
tgcacacgcgcatggagat

SEQ ID NO: 722 - DNA, 19 bp, synthetic construct
  source 1..19: mol_type = other DNA
tgcacaacgctatgctgat

SEQ ID NO: 723 - DNA, 19 bp, synthetic construct
  source 1..19: mol_type = other DNA
tgcacacggctcagatgat

SEQ ID NO: 724 - DNA, 19 bp, synthetic construct
  source 1..19: mol_type = other DNA
tgcacacgcttgatcagat

SEQ ID NO: 725 - DNA, 19 bp, synthetic construct
  source 1..19: mol_type = other DNA
tgcacacgctgccgcagat

SEQ ID NO: 726 - DNA, 19 bp, synthetic construct
  source 1..19: mol_type = other DNA
tgcacacgcactgccagat

SEQ ID NO: 727 - DNA, 21 bp, synthetic construct
  source 1..21: mol_type = other DNA
tgcacacgcagcatgcctgat

SEQ ID NO: 728 - DNA, 19 bp, synthetic construct
  source 1..19: mol_type = other DNA
tgcacaacgcatatgagat

SEQ ID NO: 729 - DNA, 19 bp, synthetic construct
  source 1..19: mol_type = other DNA
tgcacacggcatcactgat

SEQ ID NO: 730 - DNA, 21 bp, synthetic construct
  source 1..21: mol_type = other DNA
tgcacacgcaatgtgcatgat

SEQ ID NO: 731 - DNA, 19 bp, synthetic construct
  source 1..19: mol_type = other DNA
tgcacacgcatggagcgat

SEQ ID NO: 732 - DNA, 19 bp, synthetic construct
  source 1..19: mol_type = other DNA
tgcacactagcatggcgat

SEQ ID NO: 733 - DNA, 19 bp, synthetic construct
  source 1..19: mol_type = other DNA
tgcacaactatctgcagat

SEQ ID NO: 734 - DNA, 19 bp, synthetic construct
  source 1..19: mol_type = other DNA
tgcacacttatcatgtgat

SEQ ID NO: 735 - DNA, 21 bp, synthetic construct
  source 1..21: mol_type = other DNA
tgcacactattgatgcatgat

SEQ ID NO: 736 - DNA, 19 bp, synthetic construct
  source 1..19: mol_type = other DNA
tgcacactcgcaatatgat

SEQ ID NO: 737 - DNA, 19 bp, synthetic construct
  source 1..19: mol_type = other DNA
tgcacactctctcaatgat

SEQ ID NO: 738 - DNA, 19 bp, synthetic construct
  source 1..19: mol_type = other DNA
tgcacaactctcatgagat

SEQ ID NO: 739 - DNA, 19 bp, synthetic construct
  source 1..19: mol_type = other DNA
tgcacacttctgcgatgat

SEQ ID NO: 740 - DNA, 19 bp, synthetic construct
  source 1..19: mol_type = other DNA
tgcacactcttgcatcgat

SEQ ID NO: 741 - DNA, 21 bp, synthetic construct
  source 1..21: mol_type = other DNA
tgcacactcacaatgcatgat

SEQ ID NO: 742 - DNA, 19 bp, synthetic construct
  source 1..19: mol_type = other DNA
tgcacactcagcgccagat

SEQ ID NO: 743 - DNA, 19 bp, synthetic construct
  source 1..19: mol_type = other DNA
tgcacaactcatatgcgat

SEQ ID NO: 744 - DNA, 19 bp, synthetic construct
  source 1..19: mol_type = other DNA
tgcacacttcatgtatgat

SEQ ID NO: 745 - DNA, 21 bp, synthetic construct
  source 1..21: mol_type = other DNA
tgcacactcaatgctgctgat

SEQ ID NO: 746 - DNA, 21 bp, synthetic construct
  source 1..21: mol_type = other DNA
tgcacactcatggcacatgat

SEQ ID NO: 747 - DNA, 19 bp, synthetic construct
  source 1..19: mol_type = other DNA
tgcacactgtcagccagat

SEQ ID NO: 748 - DNA, 19 bp, synthetic construct
  source 1..19: mol_type = other DNA
tgcacaactgtcatctgat

SEQ ID NO: 749 - DNA, 19 bp, synthetic construct
  source 1..19: mol_type = other DNA
tgcacacttgtgctatgat

SEQ ID NO: 750 - DNA, 19 bp, synthetic construct
  source 1..19: mol_type = other DNA
tgcacactgaatgtgcgat

SEQ ID NO: 751 - DNA, 19 bp, synthetic construct
  source 1..19: mol_type = other DNA
tgcacactgatggcgtgat

SEQ ID NO: 752 - DNA, 19 bp, synthetic construct
  source 1..19: mol_type = other DNA
tgcacactgatgcaacgat

SEQ ID NO: 753 - DNA, 21 bp, synthetic construct
  source 1..21: mol_type = other DNA
tgcacactgatgcatggagat

SEQ ID NO: 754 - DNA, 19 bp, synthetic construct
  source 1..19: mol_type = other DNA
tgcacaactgcgctctgat

SEQ ID NO: 755 - DNA, 19 bp, synthetic construct
  source 1..19: mol_type = other DNA
tgcacacttgctctgtgat

SEQ ID NO: 756 - DNA, 21 bp, synthetic construct
  source 1..21: mol_type = other DNA
tgcacactgcctgatgatgat

SEQ ID NO: 757 - DNA, 21 bp, synthetic construct
  source 1..21: mol_type = other DNA
tgcacactgctggcagctgat

SEQ ID NO: 758 - DNA, 19 bp, synthetic construct
  source 1..19: mol_type = other DNA
tgcacactgcacgaatgat

SEQ ID NO: 759 - DNA, 19 bp, synthetic construct
  source 1..19: mol_type = other DNA
tgcacaactgcacatagat

SEQ ID NO: 760 - DNA, 19 bp, synthetic construct
  source 1..19: mol_type = other DNA
tgcacacttgcagatcgat

SEQ ID NO: 761 - DNA, 21 bp, synthetic construct
  source 1..21: mol_type = other DNA
tgcacactgccatatcatgat

SEQ ID NO: 762 - DNA, 19 bp, synthetic construct
  source 1..19: mol_type = other DNA
tgcacactgcattctcgat

SEQ ID NO: 763 - DNA, 21 bp, synthetic construct
  source 1..21: mol_type = other DNA
tgcacacacgcatggcatgat

SEQ ID NO: 764 - DNA, 19 bp, synthetic construct
  source 1..19: mol_type = other DNA
tgcacaacactctgcagat

SEQ ID NO: 765 - DNA, 19 bp, synthetic construct
  source 1..19: mol_type = other DNA
tgcacacaactcatatgat

SEQ ID NO: 766 - DNA, 19 bp, synthetic construct
  source 1..19: mol_type = other DNA
tgcacacacttgatgcgat

SEQ ID NO: 767 - DNA, 19 bp, synthetic construct
  source 1..19: mol_type = other DNA
tgcacacacacaacatgat

SEQ ID NO: 768 - DNA, 19 bp, synthetic construct
  source 1..19: mol_type = other DNA
tgcacacacagataatgat

SEQ ID NO: 769 - DNA, 19 bp, synthetic construct
  source 1..19: mol_type = other DNA
tgcacaacacagctgtgat

SEQ ID NO: 770 - DNA, 19 bp, synthetic construct
  source 1..19: mol_type = other DNA
tgcacacaacatatcagat

SEQ ID NO: 771 - DNA, 19 bp, synthetic construct
  source 1..19: mol_type = other DNA
tgcacacacaatatgtgat

SEQ ID NO: 772 - DNA, 19 bp, synthetic construct
  source 1..19: mol_type = other DNA
tgcacacacatccagcgat

SEQ ID NO: 773 - DNA, 21 bp, synthetic construct
  source 1..21: mol_type = other DNA
tgcacacacatgaggcatgat

SEQ ID NO: 774 - DNA, 19 bp, synthetic construct
  source 1..19: mol_type = other DNA
tgcacaacacatgctagat

SEQ ID NO: 775 - DNA, 21 bp, synthetic construct
  source 1..21: mol_type = other DNA
tgcacacaacatgcatctgat

SEQ ID NO: 776 - DNA, 19 bp, synthetic construct
  source 1..19: mol_type = other DNA
tgcacacagttatgcagat

SEQ ID NO: 777 - DNA, 19 bp, synthetic construct
  source 1..19: mol_type = other DNA
tgcacacagtcaatgtgat

SEQ ID NO: 778 - DNA, 19 bp, synthetic construct
  source 1..19: mol_type = other DNA
tgcacacagtgcgaatgat

SEQ ID NO: 779 - DNA, 19 bp, synthetic construct
  source 1..19: mol_type = other DNA
tgcacaacagtgcatagat

SEQ ID NO: 780 - DNA, 19 bp, synthetic construct
  source 1..19: mol_type = other DNA
tgcacacaagacatgagat

SEQ ID NO: 781 - DNA, 19 bp, synthetic construct
  source 1..19: mol_type = other DNA
tgcacacagaagatgcgat

SEQ ID NO: 782 - DNA, 19 bp, synthetic construct
  source 1..19: mol_type = other DNA
tgcacacagataatctgat

SEQ ID NO: 783 - DNA, 19 bp, synthetic construct
  source 1..19: mol_type = other DNA
tgcacacagatcgccagat

SEQ ID NO: 784 - DNA, 19 bp, synthetic construct
  source 1..19: mol_type = other DNA
tgcacaacagatgtatgat

SEQ ID NO: 785 - DNA, 19 bp, synthetic construct
  source 1..19: mol_type = other DNA
tgcacacaagatgacagat

SEQ ID NO: 786 - DNA, 19 bp, synthetic construct
  source 1..19: mol_type = other DNA
tgcacacagaatgctcgat

SEQ ID NO: 787 - DNA, 21 bp, synthetic construct
  source 1..21: mol_type = other DNA
tgcacacagatggcagctgat

SEQ ID NO: 788 - DNA, 19 bp, synthetic construct
  source 1..19: mol_type = other DNA
tgcacacagcgctccagat

SEQ ID NO: 789 - DNA, 19 bp, synthetic construct
  source 1..19: mol_type = other DNA
tgcacaacagctctctgat

SEQ ID NO: 790 - DNA, 19 bp, synthetic construct
  source 1..19: mol_type = other DNA
tgcacacaagctcacagat

SEQ ID NO: 791 - DNA, 19 bp, synthetic construct
  source 1..19: mol_type = other DNA
tgcacacagcctgtgtgat

SEQ ID NO: 792 - DNA, 19 bp, synthetic construct
  source 1..19: mol_type = other DNA
tgcacacagcaccgctgat

SEQ ID NO: 793 - DNA, 19 bp, synthetic construct
  source 1..19: mol_type = other DNA
tgcacacagcactaatgat

SEQ ID NO: 794 - DNA, 21 bp, synthetic construct
  source 1..21: mol_type = other DNA
tgcacacagcagcagaatgat

SEQ ID NO: 795 - DNA, 19 bp, synthetic construct
  source 1..19: mol_type = other DNA
tgcacaacatacatcagat

SEQ ID NO: 796 - DNA, 19 bp, synthetic construct
  source 1..19: mol_type = other DNA
tgcacacaatagcgatgat

SEQ ID NO: 797 - DNA, 19 bp, synthetic construct
  source 1..19: mol_type = other DNA
tgcacacataagcactgat

SEQ ID NO: 798 - DNA, 19 bp, synthetic construct
  source 1..19: mol_type = other DNA
tgcacacatataagatgat

SEQ ID NO: 799 - DNA, 19 bp, synthetic construct
  source 1..19: mol_type = other DNA
tgcacacatatctcctgat

SEQ ID NO: 800 - DNA, 19 bp, synthetic construct
  source 1..19: mol_type = other DNA
tgcacaacatatgtcagat

SEQ ID NO: 801 - DNA, 19 bp, synthetic construct
  source 1..19: mol_type = other DNA
tgcacacaatatgtgtgat

SEQ ID NO: 802 - DNA, 19 bp, synthetic construct
  source 1..19: mol_type = other DNA
tgcacacataatgagcgat

SEQ ID NO: 803 - DNA, 21 bp, synthetic construct
  source 1..21: mol_type = other DNA
tgcacacatatggcatatgat

SEQ ID NO: 804 - DNA, 21 bp, synthetic construct
  source 1..21: mol_type = other DNA
tgcacacatctatggcatgat

SEQ ID NO: 805 - DNA, 19 bp, synthetic construct
  source 1..19: mol_type = other DNA
tgcacaacatctgatagat

SEQ ID NO: 806 - DNA, 21 bp, synthetic construct
  source 1..21: mol_type = other DNA
tgcacacaatctgcagcagat

SEQ ID NO: 807 - DNA, 21 bp, synthetic construct
  source 1..21: mol_type = other DNA
tgcacacatcctgcatgtgat

SEQ ID NO: 808 - DNA, 19 bp, synthetic construct
  source 1..19: mol_type = other DNA
tgcacacatcaccagtgat

SEQ ID NO: 809 - DNA, 19 bp, synthetic construct
  source 1..19: mol_type = other DNA
tgcacacatcagtggcgat

SEQ ID NO: 810 - DNA, 21 bp, synthetic construct
  source 1..21: mol_type = other DNA
tgcacacatcagctcaatgat

SEQ ID NO: 811 - DNA, 21 bp, synthetic construct
  source 1..21: mol_type = other DNA
tgcacaacatcagcatgagat

SEQ ID NO: 812 - DNA, 21 bp, synthetic construct
  source 1..21: mol_type = other DNA
tgcacacaatcatcgcatgat

SEQ ID NO: 813 - DNA, 19 bp, synthetic construct
  source 1..19: mol_type = other DNA
tgcacacatggtacatgat

SEQ ID NO: 814 - DNA, 19 bp, synthetic construct
  source 1..19: mol_type = other DNA
tgcacacatgtcctgagat

SEQ ID NO: 815 - DNA, 19 bp, synthetic construct
  source 1..19: mol_type = other DNA
tgcacacatgtgaggagat

SEQ ID NO: 816 - DNA, 19 bp, synthetic construct
  source 1..19: mol_type = other DNA
tgcacaacatgtgatcgat

SEQ ID NO: 817 - DNA, 19 bp, synthetic construct
  source 1..19: mol_type = other DNA
tgcacacaatgtgcgcgat

SEQ ID NO: 818 - DNA, 19 bp, synthetic construct
  source 1..19: mol_type = other DNA
tgcacacatggactgtgat

SEQ ID NO: 819 - DNA, 19 bp, synthetic construct
  source 1..19: mol_type = other DNA
tgcacacatgaccagcgat

SEQ ID NO: 820 - DNA, 21 bp, synthetic construct
  source 1..21: mol_type = other DNA
tgcacacatgagtggcatgat

SEQ ID NO: 821 - DNA, 19 bp, synthetic construct
  source 1..19: mol_type = other DNA
tgcacaacatgagatagat

SEQ ID NO: 822 - DNA, 19 bp, synthetic construct
  source 1..19: mol_type = other DNA
tgcacacaatgcgagcgat

SEQ ID NO: 823 - DNA, 21 bp, synthetic construct
  source 1..21: mol_type = other DNA
tgcacacatggcgatcatgat

SEQ ID NO: 824 - DNA, 21 bp, synthetic construct
  source 1..21: mol_type = other DNA
tgcacacatgcggcgcatgat

SEQ ID NO: 825 - DNA, 21 bp, synthetic construct
  source 1..21: mol_type = other DNA
tgcacacatgctcaatgcgat

SEQ ID NO: 826 - DNA, 21 bp, synthetic construct
  source 1..21: mol_type = other DNA
tgcacacatgctgtgccagat

SEQ ID NO: 827 - DNA, 21 bp, synthetic construct
  source 1..21: mol_type = other DNA
tgcacaacatgcacatctgat

SEQ ID NO: 828 - DNA, 21 bp, synthetic construct
  source 1..21: mol_type = other DNA
tgcacacaatgcagatgtgat

SEQ ID NO: 829 - DNA, 21 bp, synthetic construct
  source 1..21: mol_type = other DNA
tgcacacatggcagcacagat

SEQ ID NO: 830 - DNA, 21 bp, synthetic construct
  source 1..21: mol_type = other DNA
tgcacacatgcaatagctgat

SEQ ID NO: 831 - DNA, 21 bp, synthetic construct
  source 1..21: mol_type = other DNA
tgcacacatgcatccagagat

SEQ ID NO: 832 - DNA, 21 bp, synthetic construct
  source 1..21: mol_type = other DNA
tgcacacatgcatgtcctgat

SEQ ID NO: 833 - DNA, 19 bp, synthetic construct
  source 1..19: mol_type = other DNA
tgcacaagtagcatcagat

SEQ ID NO: 834 - DNA, 19 bp, synthetic construct
  source 1..19: mol_type = other DNA
tgcacagttatgtgctgat

SEQ ID NO: 835 - DNA, 19 bp, synthetic construct
  source 1..19: mol_type = other DNA
tgcacagtattgctgagat

SEQ ID NO: 836 - DNA, 19 bp, synthetic construct
  source 1..19: mol_type = other DNA
tgcacagtcgcttgatgat

SEQ ID NO: 837 - DNA, 19 bp, synthetic construct
  source 1..19: mol_type = other DNA
tgcacagtctgatcctgat

SEQ ID NO: 838 - DNA, 19 bp, synthetic construct
  source 1..19: mol_type = other DNA
tgcacaagtctgctcagat

SEQ ID NO: 839 - DNA, 19 bp, synthetic construct
  source 1..19: mol_type = other DNA
tgcacagttctgcagtgat

SEQ ID NO: 840 - DNA, 19 bp, synthetic construct
  source 1..19: mol_type = other DNA
tgcacagtcaacagcagat

SEQ ID NO: 841 - DNA, 19 bp, synthetic construct
  source 1..19: mol_type = other DNA
tgcacagtcagttgcagat

SEQ ID NO: 842 - DNA, 19 bp, synthetic construct
  source 1..19: mol_type = other DNA
tgcacagtcagataatgat

SEQ ID NO: 843 - DNA, 19 bp, synthetic construct
  source 1..19: mol_type = other DNA
tgcacaagtcagcactgat

SEQ ID NO: 844 - DNA, 19 bp, synthetic construct
  source 1..19: mol_type = other DNA
tgcacagttcatctgtgat

SEQ ID NO: 845 - DNA, 19 bp, synthetic construct
  source 1..19: mol_type = other DNA
tgcacagtcaatgagcgat

SEQ ID NO: 846 - DNA, 19 bp, synthetic construct
  source 1..19: mol_type = other DNA
tgcacagtgtattgctgat

SEQ ID NO: 847 - DNA, 19 bp, synthetic construct
  source 1..19: mol_type = other DNA
tgcacagtgtcagaatgat

SEQ ID NO: 848 - DNA, 19 bp, synthetic construct
  source 1..19: mol_type = other DNA
tgcacaagtgtgcgcagat

SEQ ID NO: 849 - DNA, 19 bp, synthetic construct
  source 1..19: mol_type = other DNA
tgcacagttgtgctgtgat

SEQ ID NO: 850 - DNA, 19 bp, synthetic construct
  source 1..19: mol_type = other DNA
tgcacagtgaacgcatgat

SEQ ID NO: 851 - DNA, 19 bp, synthetic construct
  source 1..19: mol_type = other DNA
tgcacagtgacaatgtgat

SEQ ID NO: 852 - DNA, 19 bp, synthetic construct
  source 1..19: mol_type = other DNA
tgcacagtgagctaatgat

SEQ ID NO: 853 - DNA, 19 bp, synthetic construct
  source 1..19: mol_type = other DNA
tgcacaagtgagctgcgat

SEQ ID NO: 854 - DNA, 19 bp, synthetic construct
  source 1..19: mol_type = other DNA
tgcacagttgatacatgat

SEQ ID NO: 855 - DNA, 19 bp, synthetic construct
  source 1..19: mol_type = other DNA
tgcacagtgaatcatcgat

SEQ ID NO: 856 - DNA, 19 bp, synthetic construct
  source 1..19: mol_type = other DNA
tgcacagtgatggtcagat

SEQ ID NO: 857 - DNA, 19 bp, synthetic construct
  source 1..19: mol_type = other DNA
tgcacagtgcgacaatgat

SEQ ID NO: 858 - DNA, 19 bp, synthetic construct
  source 1..19: mol_type = other DNA
tgcacaagtgcgatgagat

SEQ ID NO: 859 - DNA, 21 bp, synthetic construct
  source 1..21: mol_type = other DNA
tgcacagttgcgcatgctgat

SEQ ID NO: 860 - DNA, 19 bp, synthetic construct
  source 1..19: mol_type = other DNA
tgcacagtgcctagcagat

SEQ ID NO: 861 - DNA, 19 bp, synthetic construct
  source 1..19: mol_type = other DNA
tgcacagtgctccatagat

SEQ ID NO: 862 - DNA, 21 bp, synthetic construct
  source 1..21: mol_type = other DNA
tgcacagtgctgtggcatgat

SEQ ID NO: 863 - DNA, 19 bp, synthetic construct
  source 1..19: mol_type = other DNA
tgcacaagtgcagcgagat

SEQ ID NO: 864 - DNA, 21 bp, synthetic construct
  source 1..21: mol_type = other DNA
tgcacagttgcatctgcagat

SEQ ID NO: 865 - DNA, 19 bp, synthetic construct
  source 1..19: mol_type = other DNA
tgcacagacggatgcagat

SEQ ID NO: 866 - DNA, 19 bp, synthetic construct
  source 1..19: mol_type = other DNA
tgcacagacgcaatctgat

SEQ ID NO: 867 - DNA, 19 bp, synthetic construct
  source 1..19: mol_type = other DNA
tgcacagactcacaatgat

SEQ ID NO: 868 - DNA, 19 bp, synthetic construct
  source 1..19: mol_type = other DNA
tgcacaagactgatatgat

SEQ ID NO: 869 - DNA, 19 bp, synthetic construct
  source 1..19: mol_type = other DNA
tgcacagaactgcgatgat

SEQ ID NO: 870 - DNA, 19 bp, synthetic construct
  source 1..19: mol_type = other DNA
tgcacagacttgctgcgat

SEQ ID NO: 871 - DNA, 19 bp, synthetic construct
  source 1..19: mol_type = other DNA
tgcacagacacaatatgat

SEQ ID NO: 872 - DNA, 21 bp, synthetic construct
  source 1..21: mol_type = other DNA
tgcacagacatctggcatgat

SEQ ID NO: 873 - DNA, 19 bp, synthetic construct
  source 1..19: mol_type = other DNA
tgcacaagacatcagagat

SEQ ID NO: 874 - DNA, 19 bp, synthetic construct
  source 1..19: mol_type = other DNA
tgcacagaacatgtcagat

SEQ ID NO: 875 - DNA, 19 bp, synthetic construct
  source 1..19: mol_type = other DNA
tgcacagagttcatatgat

SEQ ID NO: 876 - DNA, 19 bp, synthetic construct
  source 1..19: mol_type = other DNA
tgcacagagtgccgctgat

SEQ ID NO: 877 - DNA, 19 bp, synthetic construct
  source 1..19: mol_type = other DNA
tgcacagagacacaatgat

SEQ ID NO: 878 - DNA, 19 bp, synthetic construct
  source 1..19: mol_type = other DNA
tgcacaagagagagctgat

SEQ ID NO: 879 - DNA, 19 bp, synthetic construct
  source 1..19: mol_type = other DNA
tgcacagaagagatatgat

SEQ ID NO: 880 - DNA, 19 bp, synthetic construct
  source 1..19: mol_type = other DNA
tgcacagagaagcgatgat

SEQ ID NO: 881 - DNA, 21 bp, synthetic construct
  source 1..21: mol_type = other DNA
tgcacagagagccatgcagat

SEQ ID NO: 882 - DNA, 19 bp, synthetic construct
  source 1..19: mol_type = other DNA
tgcacagagatctggtgat

SEQ ID NO: 883 - DNA, 21 bp, synthetic construct
  source 1..21: mol_type = other DNA
tgcacagagatgtgcaatgat

SEQ ID NO: 884 - DNA, 21 bp, synthetic construct
  source 1..21: mol_type = other DNA
tgcacaagagatgcatctgat

SEQ ID NO: 885 - DNA, 19 bp, synthetic construct
  source 1..19: mol_type = other DNA
tgcacagaagcgtgctgat

SEQ ID NO: 886 - DNA, 19 bp, synthetic construct
  source 1..19: mol_type = other DNA
tgcacagagccgagatgat

SEQ ID NO: 887 - DNA, 19 bp, synthetic construct
  source 1..19: mol_type = other DNA
tgcacagagcgccgcagat

SEQ ID NO: 888 - DNA, 19 bp, synthetic construct
  source 1..19: mol_type = other DNA
tgcacagagctagcctgat

SEQ ID NO: 889 - DNA, 19 bp, synthetic construct
  source 1..19: mol_type = other DNA
tgcacaagagctgacagat

SEQ ID NO: 890 - DNA, 21 bp, synthetic construct
  source 1..21: mol_type = other DNA
tgcacagaagctgcatgagat

SEQ ID NO: 891 - DNA, 19 bp, synthetic construct
  source 1..19: mol_type = other DNA
tgcacagagccactcagat

SEQ ID NO: 892 - DNA, 19 bp, synthetic construct
  source 1..19: mol_type = other DNA
tgcacagagcaccagcgat

SEQ ID NO: 893 - DNA, 21 bp, synthetic construct
  source 1..21: mol_type = other DNA
tgcacagagcatgaatgtgat

SEQ ID NO: 894 - DNA, 21 bp, synthetic construct
  source 1..21: mol_type = other DNA
tgcacagagcatgctaatgat

SEQ ID NO: 895 - DNA, 19 bp, synthetic construct
  source 1..19: mol_type = other DNA
tgcacaagatactcatgat

SEQ ID NO: 896 - DNA, 19 bp, synthetic construct
  source 1..19: mol_type = other DNA
tgcacagaatacatgcgat

SEQ ID NO: 897 - DNA, 19 bp, synthetic construct
  source 1..19: mol_type = other DNA
tgcacagataagagcagat

SEQ ID NO: 898 - DNA, 19 bp, synthetic construct
  source 1..19: mol_type = other DNA
tgcacagatagccgctgat

SEQ ID NO: 899 - DNA, 19 bp, synthetic construct
  source 1..19: mol_type = other DNA
tgcacagatatagcctgat

SEQ ID NO: 900 - DNA, 19 bp, synthetic construct
  source 1..19: mol_type = other DNA
tgcacaagatatatatgat

SEQ ID NO: 901 - DNA, 21 bp, synthetic construct
  source 1..21: mol_type = other DNA
tgcacagaatatgcagatgat

SEQ ID NO: 902 - DNA, 19 bp, synthetic construct
  source 1..19: mol_type = other DNA
tgcacagatccgatgtgat

SEQ ID NO: 903 - DNA, 19 bp, synthetic construct
  source 1..19: mol_type = other DNA
tgcacagatcgccacagat

SEQ ID NO: 904 - DNA, 19 bp, synthetic construct
  source 1..19: mol_type = other DNA
tgcacagatctatccagat

SEQ ID NO: 905 - DNA, 21 bp, synthetic construct
  source 1..21: mol_type = other DNA
tgcacagatctcatgaatgat

SEQ ID NO: 906 - DNA, 19 bp, synthetic construct
  source 1..19: mol_type = other DNA
tgcacaagatctgagagat

SEQ ID NO: 907 - DNA, 19 bp, synthetic construct
  source 1..19: mol_type = other DNA
tgcacagaatcagtctgat

SEQ ID NO: 908 - DNA, 21 bp, synthetic construct
  source 1..21: mol_type = other DNA
tgcacagatccatcatctgat

SEQ ID NO: 909 - DNA, 21 bp, synthetic construct
  source 1..21: mol_type = other DNA
tgcacagatcattgtgatgat

SEQ ID NO: 910 - DNA, 21 bp, synthetic construct
  source 1..21: mol_type = other DNA
tgcacagatcatgccgcagat

SEQ ID NO: 911 - DNA, 19 bp, synthetic construct
  source 1..19: mol_type = other DNA
tgcacaagatgtatgagat

SEQ ID NO: 912 - DNA, 21 bp, synthetic construct
  source 1..21: mol_type = other DNA
tgcacagaatgtctgcatgat

SEQ ID NO: 913 - DNA, 19 bp, synthetic construct
  source 1..19: mol_type = other DNA
tgcacagatggtcacagat

SEQ ID NO: 914 - DNA, 21 bp, synthetic construct
  source 1..21: mol_type = other DNA
tgcacagatgtggatcatgat

SEQ ID NO: 915 - DNA, 19 bp, synthetic construct
  source 1..19: mol_type = other DNA
tgcacagatgacattagat

SEQ ID NO: 916 - DNA, 21 bp, synthetic construct
  source 1..21: mol_type = other DNA
tgcacagatgatgatggcgat

SEQ ID NO: 917 - DNA, 19 bp, synthetic construct
  source 1..19: mol_type = other DNA
tgcacaagatgctcgtgat

SEQ ID NO: 918 - DNA, 19 bp, synthetic construct
  source 1..19: mol_type = other DNA
tgcacagaatgctgtcgat

SEQ ID NO: 919 - DNA, 21 bp, synthetic construct
  source 1..21: mol_type = other DNA
tgcacagatggctgcagcgat

SEQ ID NO: 920 - DNA, 21 bp, synthetic construct
  source 1..21: mol_type = other DNA
tgcacagatgcaacagatgat

SEQ ID NO: 921 - DNA, 21 bp, synthetic construct
  source 1..21: mol_type = other DNA
tgcacagatgcatggatagat

SEQ ID NO: 922 - DNA, 21 bp, synthetic construct
  source 1..21: mol_type = other DNA
tgcacagcgtcatgcaatgat

SEQ ID NO: 923 - DNA, 19 bp, synthetic construct
  source 1..19: mol_type = other DNA
tgcacaagcgacatgcgat

SEQ ID NO: 924 - DNA, 19 bp, synthetic construct
  source 1..19: mol_type = other DNA
tgcacagccgatatcagat

SEQ ID NO: 925 - DNA, 21 bp, synthetic construct
  source 1..21: mol_type = other DNA
tgcacagcgaatgatgatgat

SEQ ID NO: 926 - DNA, 19 bp, synthetic construct
  source 1..19: mol_type = other DNA
tgcacagcgatggcgcgat

SEQ ID NO: 927 - DNA, 21 bp, synthetic construct
  source 1..21: mol_type = other DNA
tgcacagcgcgctggcatgat

SEQ ID NO: 928 - DNA, 19 bp, synthetic construct
  source 1..19: mol_type = other DNA
tgcacaagcgctctgagat

SEQ ID NO: 929 - DNA, 19 bp, synthetic construct
  source 1..19: mol_type = other DNA
tgcacagccgctgctcgat

SEQ ID NO: 930 - DNA, 21 bp, synthetic construct
  source 1..21: mol_type = other DNA
tgcacagcgcctgcatgtgat

SEQ ID NO: 931 - DNA, 21 bp, synthetic construct
  source 1..21: mol_type = other DNA
tgcacagcgcaccagcatgat

SEQ ID NO: 932 - DNA, 19 bp, synthetic construct
  source 1..19: mol_type = other DNA
tgcacagcgcataggtgat

SEQ ID NO: 933 - DNA, 21 bp, synthetic construct
  source 1..21: mol_type = other DNA
tgcacagcgcatgatcctgat

SEQ ID NO: 934 - DNA, 21 bp, synthetic construct
  source 1..21: mol_type = other DNA
tgcacaagcgcatgcgatgat

SEQ ID NO: 935 - DNA, 21 bp, synthetic construct
  source 1..21: mol_type = other DNA
tgcacagccgcatgcacagat

SEQ ID NO: 936 - DNA, 21 bp, synthetic construct
  source 1..21: mol_type = other DNA
tgcacagctaagcagcatgat

SEQ ID NO: 937 - DNA, 19 bp, synthetic construct
  source 1..19: mol_type = other DNA
tgcacagctcgaatgcgat

SEQ ID NO: 938 - DNA, 21 bp, synthetic construct
  source 1..21: mol_type = other DNA
tgcacagctctcaggcatgat

SEQ ID NO: 939 - DNA, 19 bp, synthetic construct
  source 1..19: mol_type = other DNA
tgcacaagctcactgagat

SEQ ID NO: 940 - DNA, 19 bp, synthetic construct
  source 1..19: mol_type = other DNA
tgcacagcctcagcgtgat

SEQ ID NO: 941 - DNA, 21 bp, synthetic construct
  source 1..21: mol_type = other DNA
tgcacagctccatcatcagat

SEQ ID NO: 942 - DNA, 21 bp, synthetic construct
  source 1..21: mol_type = other DNA
tgcacagctcattgcagagat

SEQ ID NO: 943 - DNA, 19 bp, synthetic construct
  source 1..19: mol_type = other DNA
tgcacagctgtgaccagat

SEQ ID NO: 944 - DNA, 19 bp, synthetic construct
  source 1..19: mol_type = other DNA
tgcacaagctgactcagat

SEQ ID NO: 945 - DNA, 21 bp, synthetic construct
  source 1..21: mol_type = other DNA
tgcacagcctgatctgctgat

SEQ ID NO: 946 - DNA, 21 bp, synthetic construct
  source 1..21: mol_type = other DNA
tgcacagctggatgagctgat

SEQ ID NO: 947 - DNA, 19 bp, synthetic construct
  source 1..19: mol_type = other DNA
tgcacagctgcggctagat

SEQ ID NO: 948 - DNA, 19 bp, synthetic construct
  source 1..19: mol_type = other DNA
tgcacagctgctacctgat

SEQ ID NO: 949 - DNA, 21 bp, synthetic construct
  source 1..21: mol_type = other DNA
tgcacagctgcagtgaatgat

SEQ ID NO: 950 - DNA, 21 bp, synthetic construct
  source 1..21: mol_type = other DNA
tgcacaagctgcagagcagat

SEQ ID NO: 951 - DNA, 21 bp, synthetic construct
  source 1..21: mol_type = other DNA
tgcacagcctgcatctatgat

SEQ ID NO: 952 - DNA, 21 bp, synthetic construct
  source 1..21: mol_type = other DNA
tgcacagcacctgcatcagat

SEQ ID NO: 953 - DNA, 21 bp, synthetic construct
  source 1..21: mol_type = other DNA
tgcacagcacaccatgcagat

SEQ ID NO: 954 - DNA, 19 bp, synthetic construct
  source 1..19: mol_type = other DNA
tgcacagcacagaggagat

SEQ ID NO: 955 - DNA, 21 bp, synthetic construct
  source 1..21: mol_type = other DNA
tgcacagcacatgcgcctgat

SEQ ID NO: 956 - DNA, 19 bp, synthetic construct
  source 1..19: mol_type = other DNA
tgcacaagcagtgagcgat

SEQ ID NO: 957 - DNA, 21 bp, synthetic construct
  source 1..21: mol_type = other DNA
tgcacagccagtgctcatgat

SEQ ID NO: 958 - DNA, 19 bp, synthetic construct
  source 1..19: mol_type = other DNA
tgcacagcaggagctagat

SEQ ID NO: 959 - DNA, 19 bp, synthetic construct
  source 1..19: mol_type = other DNA
tgcacagcagattcacgat

SEQ ID NO: 960 - DNA, 21 bp, synthetic construct
  source 1..21: mol_type = other DNA
tgcacagcagatcaagatgat

SEQ ID NO: 961 - DNA, 19 bp, synthetic construct
  source 1..19: mol_type = other DNA
tgcacaagcagcgatcgat

SEQ ID NO: 962 - DNA, 21 bp, synthetic construct
  source 1..21: mol_type = other DNA
tgcacagccagcgcagctgat

SEQ ID NO: 963 - DNA, 19 bp, synthetic construct
  source 1..19: mol_type = other DNA
tgcacagcaggctatagat

SEQ ID NO: 964 - DNA, 19 bp, synthetic construct
  source 1..19: mol_type = other DNA
tgcacagcagcttcgcgat

SEQ ID NO: 965 - DNA, 21 bp, synthetic construct
  source 1..21: mol_type = other DNA
tgcacagcagcagttgcagat

SEQ ID NO: 966 - DNA, 21 bp, synthetic construct
  source 1..21: mol_type = other DNA
tgcacagcagcatagccagat

SEQ ID NO: 967 - DNA, 19 bp, synthetic construct
  source 1..19: mol_type = other DNA
tgcacaagcatagtatgat

SEQ ID NO: 968 - DNA, 19 bp, synthetic construct
  source 1..19: mol_type = other DNA
tgcacagccatagatcgat

SEQ ID NO: 969 - DNA, 21 bp, synthetic construct
  source 1..21: mol_type = other DNA
tgcacagcattagcatgtgat

SEQ ID NO: 970 - DNA, 19 bp, synthetic construct
  source 1..19: mol_type = other DNA
tgcacagcatattacagat

SEQ ID NO: 971 - DNA, 19 bp, synthetic construct
  source 1..19: mol_type = other DNA
tgcacagcatatcggtgat

SEQ ID NO: 972 - DNA, 19 bp, synthetic construct
  source 1..19: mol_type = other DNA
tgcacaagcatatctagat

SEQ ID NO: 973 - DNA, 21 bp, synthetic construct
  source 1..21: mol_type = other DNA
tgcacagccatatgatgagat

SEQ ID NO: 974 - DNA, 21 bp, synthetic construct
  source 1..21: mol_type = other DNA
tgcacagcattatgctgcgat

SEQ ID NO: 975 - DNA, 19 bp, synthetic construct
  source 1..19: mol_type = other DNA
tgcacagcatcggctcgat

SEQ ID NO: 976 - DNA, 21 bp, synthetic construct
  source 1..21: mol_type = other DNA
tgcacagcatcgcaagatgat

SEQ ID NO: 977 - DNA, 21 bp, synthetic construct
  source 1..21: mol_type = other DNA
tgcacagcatctctgcctgat

SEQ ID NO: 978 - DNA, 19 bp, synthetic construct
  source 1..19: mol_type = other DNA
tgcacaagcatctgacgat

SEQ ID NO: 979 - DNA, 21 bp, synthetic construct
  source 1..21: mol_type = other DNA
tgcacagccatctgctgagat

SEQ ID NO: 980 - DNA, 21 bp, synthetic construct
  source 1..21: mol_type = other DNA
tgcacagcattcagacatgat

SEQ ID NO: 981 - DNA, 21 bp, synthetic construct
  source 1..21: mol_type = other DNA
tgcacagcatcaagcagcgat

SEQ ID NO: 982 - DNA, 21 bp, synthetic construct
  source 1..21: mol_type = other DNA
tgcacagcatgtgaatgtgat

SEQ ID NO: 983 - DNA, 21 bp, synthetic construct
  source 1..21: mol_type = other DNA
tgcacagcatgagcgccagat

SEQ ID NO: 984 - DNA, 21 bp, synthetic construct
  source 1..21: mol_type = other DNA
tgcacaagcatgctctcagat

SEQ ID NO: 985 - DNA, 21 bp, synthetic construct
  source 1..21: mol_type = other DNA
tgcacagccatgcactgcgat

SEQ ID NO: 986 - DNA, 19 bp, synthetic construct
  source 1..19: mol_type = other DNA
tgcacatacggcatgcgat

SEQ ID NO: 987 - DNA, 19 bp, synthetic construct
  source 1..19: mol_type = other DNA
tgcacatactgcctatgat

SEQ ID NO: 988 - DNA, 19 bp, synthetic construct
  source 1..19: mol_type = other DNA
tgcacatactgcaggagat

SEQ ID NO: 989 - DNA, 19 bp, synthetic construct
  source 1..19: mol_type = other DNA
tgcacaatacacatctgat

SEQ ID NO: 990 - DNA, 19 bp, synthetic construct
  source 1..19: mol_type = other DNA
tgcacataacagtgcagat

SEQ ID NO: 991 - DNA, 19 bp, synthetic construct
  source 1..19: mol_type = other DNA
tgcacatacaagagctgat

SEQ ID NO: 992 - DNA, 19 bp, synthetic construct
  source 1..19: mol_type = other DNA
tgcacatacagccgatgat

SEQ ID NO: 993 - DNA, 19 bp, synthetic construct
  source 1..19: mol_type = other DNA
tgcacatacatagaatgat

SEQ ID NO: 994 - DNA, 19 bp, synthetic construct
  source 1..19: mol_type = other DNA
tgcacaatacatgatcgat

SEQ ID NO: 995 - DNA, 21 bp, synthetic construct
  source 1..21: mol_type = other DNA
tgcacataacatgctgctgat

SEQ ID NO: 996 - DNA, 19 bp, synthetic construct
  source 1..19: mol_type = other DNA
tgcacatagttcatctgat

SEQ ID NO: 997 - DNA, 19 bp, synthetic construct
  source 1..19: mol_type = other DNA
tgcacatagtgaatatgat

SEQ ID NO: 998 - DNA, 19 bp, synthetic construct
  source 1..19: mol_type = other DNA
tgcacatagtgatggcgat

SEQ ID NO: 999 - DNA, 21 bp, synthetic construct
  source 1..21: mol_type = other DNA
tgcacatagtgctgcaatgat

SEQ ID NO: 1000 - DNA, 19 bp, synthetic construct
  source 1..19: mol_type = other DNA
tgcacaatagacagctgat

SEQ ID NO: 1001 - DNA, 19 bp, synthetic construct
  source 1..19: mol_type = other DNA
tgcacataagacatatgat

SEQ ID NO: 1002 - DNA, 19 bp, synthetic construct
  source 1..19: mol_type = other DNA
tgcacatagaagatgtgat

SEQ ID NO: 1003 - DNA, 19 bp, synthetic construct
  source 1..19: mol_type = other DNA
tgcacatagagcctcagat

SEQ ID NO: 1004 - DNA, 19 bp, synthetic construct
  source 1..19: mol_type = other DNA
tgcacatagatagccagat

SEQ ID NO: 1005 - DNA, 19 bp, synthetic construct
  source 1..19: mol_type = other DNA
tgcacaatagatgtgagat

SEQ ID NO: 1006 - DNA, 19 bp, synthetic construct
  source 1..19: mol_type = other DNA
tgcacataagatgcgtgat

SEQ ID NO: 1007 - DNA, 19 bp, synthetic construct
  source 1..19: mol_type = other DNA
tgcacatagaatgcacgat

SEQ ID NO: 1008 - DNA, 19 bp, synthetic construct
  source 1..19: mol_type = other DNA
tgcacatagcgttcatgat

SEQ ID NO: 1009 - DNA, 19 bp, synthetic construct
  source 1..19: mol_type = other DNA
tgcacatagcgagccagat

SEQ ID NO: 1010 - DNA, 19 bp, synthetic construct
  source 1..19: mol_type = other DNA
tgcacaatagctatgagat

SEQ ID NO: 1011 - DNA, 19 bp, synthetic construct
  source 1..19: mol_type = other DNA
tgcacataagctcagtgat

SEQ ID NO: 1012 - DNA, 19 bp, synthetic construct
  source 1..19: mol_type = other DNA
tgcacatagcctgactgat

SEQ ID NO: 1013 - DNA, 21 bp, synthetic construct
  source 1..21: mol_type = other DNA
tgcacatagctggcatcagat

SEQ ID NO: 1014 - DNA, 21 bp, synthetic construct
  source 1..21: mol_type = other DNA
tgcacatagcagcaacatgat

SEQ ID NO: 1015 - DNA, 19 bp, synthetic construct
  source 1..19: mol_type = other DNA
tgcacaatagcatcgagat

SEQ ID NO: 1016 - DNA, 21 bp, synthetic construct
  source 1..21: mol_type = other DNA
tgcacataagcatctcatgat

SEQ ID NO: 1017 - DNA, 21 bp, synthetic construct
  source 1..21: mol_type = other DNA
tgcacatataacatgcatgat

SEQ ID NO: 1018 - DNA, 19 bp, synthetic construct
  source 1..19: mol_type = other DNA
tgcacatatagcctatgat

SEQ ID NO: 1019 - DNA, 19 bp, synthetic construct
  source 1..19: mol_type = other DNA
tgcacatatagcaggagat

SEQ ID NO: 1020 - DNA, 19 bp, synthetic construct
  source 1..19: mol_type = other DNA
tgcacaatatatatgtgat

SEQ ID NO: 1021 - DNA, 19 bp, synthetic construct
  source 1..19: mol_type = other DNA
tgcacataatatctgcgat

SEQ ID NO: 1022 - DNA, 19 bp, synthetic construct
  source 1..19: mol_type = other DNA
tgcacatataatcacagat

SEQ ID NO: 1023 - DNA, 21 bp, synthetic construct
  source 1..21: mol_type = other DNA
tgcacatatatggtgcatgat

SEQ ID NO: 1024 - DNA, 19 bp, synthetic construct
  source 1..19: mol_type = other DNA
tgcacatatatgacctgat

SEQ ID NO: 1025 - DNA, 19 bp, synthetic construct
  source 1..19: mol_type = other DNA
tgcacaatatcgatatgat

SEQ ID NO: 1026 - DNA, 19 bp, synthetic construct
  source 1..19: mol_type = other DNA
tgcacataatcgcgctgat

SEQ ID NO: 1027 - DNA, 19 bp, synthetic construct
  source 1..19: mol_type = other DNA
tgcacatatcctcgcagat

SEQ ID NO: 1028 - DNA, 19 bp, synthetic construct
  source 1..19: mol_type = other DNA
tgcacatatctcctgtgat

SEQ ID NO: 1029 - DNA, 19 bp, synthetic construct
  source 1..19: mol_type = other DNA
tgcacatatctgtccagat

SEQ ID NO: 1030 - DNA, 19 bp, synthetic construct
  source 1..19: mol_type = other DNA
tgcacaatatctgagtgat

SEQ ID NO: 1031 - DNA, 21 bp, synthetic construct
  source 1..21: mol_type = other DNA
tgcacataatctgcacatgat

SEQ ID NO: 1032 - DNA, 19 bp, synthetic construct
  source 1..19: mol_type = other DNA
tgcacatatccacagcgat

SEQ ID NO: 1033 - DNA, 21 bp, synthetic construct
  source 1..21: mol_type = other DNA
tgcacatatcattatcatgat

SEQ ID NO: 1034 - DNA, 19 bp, synthetic construct
  source 1..19: mol_type = other DNA
tgcacatatcatcttagat

SEQ ID NO: 1035 - DNA, 21 bp, synthetic construct
  source 1..21: mol_type = other DNA
tgcacatatcatgagccagat

SEQ ID NO: 1036 - DNA, 19 bp, synthetic construct
  source 1..19: mol_type = other DNA
tgcacaatatgtcgatgat

SEQ ID NO: 1037 - DNA, 19 bp, synthetic construct
  source 1..19: mol_type = other DNA
tgcacataatgtcagcgat

SEQ ID NO: 1038 - DNA, 19 bp, synthetic construct
  source 1..19: mol_type = other DNA
tgcacatatggtgacagat

SEQ ID NO: 1039 - DNA, 19 bp, synthetic construct
  source 1..19: mol_type = other DNA
tgcacatatgacctgagat

SEQ ID NO: 1040 - DNA, 19 bp, synthetic construct
  source 1..19: mol_type = other DNA
tgcacatatgagattcgat

SEQ ID NO: 1041 - DNA, 21 bp, synthetic construct
  source 1..21: mol_type = other DNA
tgcacatatgatgagaatgat

SEQ ID NO: 1042 - DNA, 21 bp, synthetic construct
  source 1..21: mol_type = other DNA
tgcacaatatgatgcatagat

SEQ ID NO: 1043 - DNA, 19 bp, synthetic construct
  source 1..19: mol_type = other DNA
tgcacataatgcgtgagat

SEQ ID NO: 1044 - DNA, 19 bp, synthetic construct
  source 1..19: mol_type = other DNA
tgcacatatggcgcacgat

SEQ ID NO: 1045 - DNA, 21 bp, synthetic construct
  source 1..21: mol_type = other DNA
tgcacatatgcggcagatgat

SEQ ID NO: 1046 - DNA, 21 bp, synthetic construct
  source 1..21: mol_type = other DNA
tgcacatatgctgttgctgat

SEQ ID NO: 1047 - DNA, 19 bp, synthetic construct
  source 1..19: mol_type = other DNA
tgcacaatatgcacgtgat

SEQ ID NO: 1048 - DNA, 21 bp, synthetic construct
  source 1..21: mol_type = other DNA
tgcacataatgcagctgcgat

SEQ ID NO: 1049 - DNA, 21 bp, synthetic construct
  source 1..21: mol_type = other DNA
tgcacatatggcatatgcgat

SEQ ID NO: 1050 - DNA, 21 bp, synthetic construct
  source 1..21: mol_type = other DNA
tgcacatcgagccatgcagat

SEQ ID NO: 1051 - DNA, 21 bp, synthetic construct
  source 1..21: mol_type = other DNA
tgcacatcgatcattcatgat

SEQ ID NO: 1052 - DNA, 21 bp, synthetic construct
  source 1..21: mol_type = other DNA
tgcacatcgatgcagaatgat

SEQ ID NO: 1053 - DNA, 19 bp, synthetic construct
  source 1..19: mol_type = other DNA
tgcacaatcgctctatgat

SEQ ID NO: 1054 - DNA, 19 bp, synthetic construct
  source 1..19: mol_type = other DNA
tgcacatccgctcatcgat

SEQ ID NO: 1055 - DNA, 21 bp, synthetic construct
  source 1..21: mol_type = other DNA
tgcacatcgcctgctgctgat

SEQ ID NO: 1056 - DNA, 19 bp, synthetic construct
  source 1..19: mol_type = other DNA
tgcacatcgcaccagagat

SEQ ID NO: 1057 - DNA, 19 bp, synthetic construct
  source 1..19: mol_type = other DNA
tgcacatcgcagaggtgat

SEQ ID NO: 1058 - DNA, 21 bp, synthetic construct
  source 1..21: mol_type = other DNA
tgcacatcgcagctgaatgat

SEQ ID NO: 1059 - DNA, 19 bp, synthetic construct
  source 1..19: mol_type = other DNA
tgcacaatcgcatcgtgat

SEQ ID NO: 1060 - DNA, 21 bp, synthetic construct
  source 1..21: mol_type = other DNA
tgcacatccgcatgcatagat

SEQ ID NO: 1061 - DNA, 19 bp, synthetic construct
  source 1..19: mol_type = other DNA
tgcacatctaacacatgat

SEQ ID NO: 1062 - DNA, 19 bp, synthetic construct
  source 1..19: mol_type = other DNA
tgcacatctagccatagat

SEQ ID NO: 1063 - DNA, 19 bp, synthetic construct
  source 1..19: mol_type = other DNA
tgcacatctatcaggcgat

SEQ ID NO: 1064 - DNA, 19 bp, synthetic construct
  source 1..19: mol_type = other DNA
tgcacaatctatgatcgat

SEQ ID NO: 1065 - DNA, 21 bp, synthetic construct
  source 1..21: mol_type = other DNA
tgcacatcctatgctcatgat

SEQ ID NO: 1066 - DNA, 21 bp, synthetic construct
  source 1..21: mol_type = other DNA
tgcacatctcctgatcatgat

SEQ ID NO: 1067 - DNA, 21 bp, synthetic construct
  source 1..21: mol_type = other DNA
tgcacatctctggctgcagat

SEQ ID NO: 1068 - DNA, 19 bp, synthetic construct
  source 1..19: mol_type = other DNA
tgcacatctcactggtgat

SEQ ID NO: 1069 - DNA, 21 bp, synthetic construct
  source 1..21: mol_type = other DNA
tgcacatctcagtgcaatgat

SEQ ID NO: 1070 - DNA, 21 bp, synthetic construct
  source 1..21: mol_type = other DNA
tgcacaatctcagcagatgat

SEQ ID NO: 1071 - DNA, 21 bp, synthetic construct
  source 1..21: mol_type = other DNA
tgcacatcctcagcatctgat

SEQ ID NO: 1072 - DNA, 19 bp, synthetic construct
  source 1..19: mol_type = other DNA
tgcacatctccatagagat

SEQ ID NO: 1073 - DNA, 21 bp, synthetic construct
  source 1..21: mol_type = other DNA
tgcacatctcattgatgtgat

SEQ ID NO: 1074 - DNA, 19 bp, synthetic construct
  source 1..19: mol_type = other DNA
tgcacatctgtcattagat

SEQ ID NO: 1075 - DNA, 19 bp, synthetic construct
  source 1..19: mol_type = other DNA
tgcacaatctgtgagcgat

SEQ ID NO: 1076 - DNA, 21 bp, synthetic construct
  source 1..21: mol_type = other DNA
tgcacatcctgtgcgcatgat

SEQ ID NO: 1077 - DNA, 21 bp, synthetic construct
  source 1..21: mol_type = other DNA
tgcacatctggtgcatgtgat

SEQ ID NO: 1078 - DNA, 21 bp, synthetic construct
  source 1..21: mol_type = other DNA
tgcacatctgaggatcatgat

SEQ ID NO: 1079 - DNA, 19 bp, synthetic construct
  source 1..19: mol_type = other DNA
tgcacatctgagcggagat

SEQ ID NO: 1080 - DNA, 21 bp, synthetic construct
  source 1..21: mol_type = other DNA
tgcacatctgagctgcctgat

SEQ ID NO: 1081 - DNA, 21 bp, synthetic construct
  source 1..21: mol_type = other DNA
tgcacaatctgatatgatgat

SEQ ID NO: 1082 - DNA, 19 bp, synthetic construct
  source 1..19: mol_type = other DNA
tgcacatcctgcgatagat

SEQ ID NO: 1083 - DNA, 21 bp, synthetic construct
  source 1..21: mol_type = other DNA
tgcacatctggcgatgctgat

SEQ ID NO: 1084 - DNA, 21 bp, synthetic construct
  source 1..21: mol_type = other DNA
tgcacatctgcggcacatgat

SEQ ID NO: 1085 - DNA, 19 bp, synthetic construct
  source 1..19: mol_type = other DNA
tgcacatctgctgttcgat

SEQ ID NO: 1086 - DNA, 21 bp, synthetic construct
  source 1..21: mol_type = other DNA
tgcacatctgcacatggcgat

SEQ ID NO: 1087 - DNA, 19 bp, synthetic construct
  source 1..19: mol_type = other DNA
tgcacaatctgcatacgat

SEQ ID NO: 1088 - DNA, 21 bp, synthetic construct
  source 1..21: mol_type = other DNA
tgcacatcctgcatcgcagat

SEQ ID NO: 1089 - DNA, 21 bp, synthetic construct
  source 1..21: mol_type = other DNA
tgcacatcacctcagcatgat

SEQ ID NO: 1090 - DNA, 21 bp, synthetic construct
  source 1..21: mol_type = other DNA
tgcacatcactggtgcatgat

SEQ ID NO: 1091 - DNA, 19 bp, synthetic construct
  source 1..19: mol_type = other DNA
tgcacatcactgcaacgat

SEQ ID NO: 1092 - DNA, 21 bp, synthetic construct
  source 1..21: mol_type = other DNA
tgcacatcacacatgaatgat

SEQ ID NO: 1093 - DNA, 21 bp, synthetic construct
  source 1..21: mol_type = other DNA
tgcacaatcacagcagcagat

SEQ ID NO: 1094 - DNA, 21 bp, synthetic construct
  source 1..21: mol_type = other DNA
tgcacatccacatgcagtgat

SEQ ID NO: 1095 - DNA, 19 bp, synthetic construct
  source 1..19: mol_type = other DNA
tgcacatcaggtagctgat

SEQ ID NO: 1096 - DNA, 19 bp, synthetic construct
  source 1..19: mol_type = other DNA
tgcacatcagtcctgcgat

SEQ ID NO: 1097 - DNA, 19 bp, synthetic construct
  source 1..19: mol_type = other DNA
tgcacatcagatattagat

SEQ ID NO: 1098 - DNA, 19 bp, synthetic construct
  source 1..19: mol_type = other DNA
tgcacaatcagcgcgagat

SEQ ID NO: 1099 - DNA, 21 bp, synthetic construct
  source 1..21: mol_type = other DNA
tgcacatccagcgcatgtgat

SEQ ID NO: 1100 - DNA, 21 bp, synthetic construct
  source 1..21: mol_type = other DNA
tgcacatcaggctatcatgat

SEQ ID NO: 1101 - DNA, 19 bp, synthetic construct
  source 1..19: mol_type = other DNA
tgcacatcagcttgtagat

SEQ ID NO: 1102 - DNA, 21 bp, synthetic construct
  source 1..21: mol_type = other DNA
tgcacatcagctgaagatgat

SEQ ID NO: 1103 - DNA, 21 bp, synthetic construct
  source 1..21: mol_type = other DNA
tgcacatcagcacatccagat

SEQ ID NO: 1104 - DNA, 19 bp, synthetic construct
  source 1..19: mol_type = other DNA
tgcacaatcagcagacgat

SEQ ID NO: 1105 - DNA, 21 bp, synthetic construct
  source 1..21: mol_type = other DNA
tgcacatccatagatgatgat

SEQ ID NO: 1106 - DNA, 19 bp, synthetic construct
  source 1..19: mol_type = other DNA
tgcacatcattagcgcgat

SEQ ID NO: 1107 - DNA, 21 bp, synthetic construct
  source 1..21: mol_type = other DNA
tgcacatcatcggagcatgat

SEQ ID NO: 1108 - DNA, 19 bp, synthetic construct
  source 1..19: mol_type = other DNA
tgcacatcatcgattcgat

SEQ ID NO: 1109 - DNA, 19 bp, synthetic construct
  source 1..19: mol_type = other DNA
tgcacaatcatcgctagat

SEQ ID NO: 1110 - DNA, 21 bp, synthetic construct
  source 1..21: mol_type = other DNA
tgcacatccatctcatctgat

SEQ ID NO: 1111 - DNA, 21 bp, synthetic construct
  source 1..21: mol_type = other DNA
tgcacatcattcactgcagat

SEQ ID NO: 1112 - DNA, 21 bp, synthetic construct
  source 1..21: mol_type = other DNA
tgcacatcatcaatgtgagat

SEQ ID NO: 1113 - DNA, 21 bp, synthetic construct
  source 1..21: mol_type = other DNA
tgcacatcatcatggctcgat

SEQ ID NO: 1114 - DNA, 21 bp, synthetic construct
  source 1..21: mol_type = other DNA
tgcacatcatgtctcaatgat

SEQ ID NO: 1115 - DNA, 21 bp, synthetic construct
  source 1..21: mol_type = other DNA
tgcacaatcatgtgcactgat

SEQ ID NO: 1116 - DNA, 21 bp, synthetic construct
  source 1..21: mol_type = other DNA
tgcacatccatgacgcatgat

SEQ ID NO: 1117 - DNA, 21 bp, synthetic construct
  source 1..21: mol_type = other DNA
tgcacatcattgatcatcgat

SEQ ID NO: 1118 - DNA, 21 bp, synthetic construct
  source 1..21: mol_type = other DNA
tgcacatcatgcctatgtgat

SEQ ID NO: 1119 - DNA, 21 bp, synthetic construct
  source 1..21: mol_type = other DNA
tgcacatcatgctccgctgat

SEQ ID NO: 1120 - DNA, 19 bp, synthetic construct
  source 1..19: mol_type = other DNA
tgcacaatgtactgatgat

SEQ ID NO: 1121 - DNA, 19 bp, synthetic construct
  source 1..19: mol_type = other DNA
tgcacatggtagagatgat

SEQ ID NO: 1122 - DNA, 19 bp, synthetic construct
  source 1..19: mol_type = other DNA
tgcacatgtaatatcagat

SEQ ID NO: 1123 - DNA, 19 bp, synthetic construct
  source 1..19: mol_type = other DNA
tgcacatgtatcctctgat

SEQ ID NO: 1124 - DNA, 19 bp, synthetic construct
  source 1..19: mol_type = other DNA
tgcacatgtatcaggtgat

SEQ ID NO: 1125 - DNA, 21 bp, synthetic construct
  source 1..21: mol_type = other DNA
tgcacatgtatgcgcaatgat

SEQ ID NO: 1126 - DNA, 21 bp, synthetic construct
  source 1..21: mol_type = other DNA
tgcacaatgtatgcatatgat

SEQ ID NO: 1127 - DNA, 21 bp, synthetic construct
  source 1..21: mol_type = other DNA
tgcacatggtcgatgcatgat

SEQ ID NO: 1128 - DNA, 19 bp, synthetic construct
  source 1..19: mol_type = other DNA
tgcacatgtccgcagagat

SEQ ID NO: 1129 - DNA, 19 bp, synthetic construct
  source 1..19: mol_type = other DNA
tgcacatgtctaagatgat

SEQ ID NO: 1130 - DNA, 19 bp, synthetic construct
  source 1..19: mol_type = other DNA
tgcacatgtctctaatgat

SEQ ID NO: 1131 - DNA, 19 bp, synthetic construct
  source 1..19: mol_type = other DNA
tgcacaatgtctctgcgat

SEQ ID NO: 1132 - DNA, 19 bp, synthetic construct
  source 1..19: mol_type = other DNA
tgcacatggtctgacagat

SEQ ID NO: 1133 - DNA, 21 bp, synthetic construct
  source 1..21: mol_type = other DNA
tgcacatgtccacatgctgat

SEQ ID NO: 1134 - DNA, 21 bp, synthetic construct
  source 1..21: mol_type = other DNA
tgcacatgtcattcatgagat

SEQ ID NO: 1135 - DNA, 21 bp, synthetic construct
  source 1..21: mol_type = other DNA
tgcacatgtgtgattgatgat

SEQ ID NO: 1136 - DNA, 19 bp, synthetic construct
  source 1..19: mol_type = other DNA
tgcacaatgtgtgctagat

SEQ ID NO: 1137 - DNA, 19 bp, synthetic construct
  source 1..19: mol_type = other DNA
tgcacatggtgtgcacgat

SEQ ID NO: 1138 - DNA, 19 bp, synthetic construct
  source 1..19: mol_type = other DNA
tgcacatgtggacacagat

SEQ ID NO: 1139 - DNA, 21 bp, synthetic construct
  source 1..21: mol_type = other DNA
tgcacatgtgaggatgcagat

SEQ ID NO: 1140 - DNA, 19 bp, synthetic construct
  source 1..19: mol_type = other DNA
tgcacatgtgagcggtgat

SEQ ID NO: 1141 - DNA, 21 bp, synthetic construct
  source 1..21: mol_type = other DNA
tgcacatgtgatgcaggagat

SEQ ID NO: 1142 - DNA, 19 bp, synthetic construct
  source 1..19: mol_type = other DNA
tgcacaatgtgcgagcgat

SEQ ID NO: 1143 - DNA, 21 bp, synthetic construct
  source 1..21: mol_type = other DNA
tgcacatggtgcgctcatgat

SEQ ID NO: 1144 - DNA, 21 bp, synthetic construct
  source 1..21: mol_type = other DNA
tgcacatgtggctatcatgat

SEQ ID NO: 1145 - DNA, 21 bp, synthetic construct
  source 1..21: mol_type = other DNA
tgcacatgtgcttcgcatgat

SEQ ID NO: 1146 - DNA, 21 bp, synthetic construct
  source 1..21: mol_type = other DNA
tgcacatgtgcagccatcgat

SEQ ID NO: 1147 - DNA, 21 bp, synthetic construct
  source 1..21: mol_type = other DNA
tgcacatgtgcatatggtgat

SEQ ID NO: 1148 - DNA, 21 bp, synthetic construct
  source 1..21: mol_type = other DNA
tgcacaatgtgcatcagcgat

SEQ ID NO: 1149 - DNA, 21 bp, synthetic construct
  source 1..21: mol_type = other DNA
tgcacatggtgcatgtgagat

SEQ ID NO: 1150 - DNA, 19 bp, synthetic construct
  source 1..19: mol_type = other DNA
tgcacatgaccgctgtgat

SEQ ID NO: 1151 - DNA, 21 bp, synthetic construct
  source 1..21: mol_type = other DNA
tgcacatgacgccagcatgat

SEQ ID NO: 1152 - DNA, 19 bp, synthetic construct
  source 1..19: mol_type = other DNA
tgcacatgacgcattagat

SEQ ID NO: 1153 - DNA, 19 bp, synthetic construct
  source 1..19: mol_type = other DNA
tgcacaatgactatctgat

SEQ ID NO: 1154 - DNA, 19 bp, synthetic construct
  source 1..19: mol_type = other DNA
tgcacatggactcagcgat

SEQ ID NO: 1155 - DNA, 19 bp, synthetic construct
  source 1..19: mol_type = other DNA
tgcacatgaccacgcagat

SEQ ID NO: 1156 - DNA, 19 bp, synthetic construct
  source 1..19: mol_type = other DNA
tgcacatgacaccagtgat

SEQ ID NO: 1157 - DNA, 19 bp, synthetic construct
  source 1..19: mol_type = other DNA
tgcacatgacagtaatgat

SEQ ID NO: 1158 - DNA, 19 bp, synthetic construct
  source 1..19: mol_type = other DNA
tgcacaatgacagctcgat

SEQ ID NO: 1159 - DNA, 21 bp, synthetic construct
  source 1..21: mol_type = other DNA
tgcacatggacatatgcagat

SEQ ID NO: 1160 - DNA, 21 bp, synthetic construct
  source 1..21: mol_type = other DNA
tgcacatgaccatgacatgat

SEQ ID NO: 1161 - DNA, 21 bp, synthetic construct
  source 1..21: mol_type = other DNA
tgcacatgagtaatgcatgat

SEQ ID NO: 1162 - DNA, 19 bp, synthetic construct
  source 1..19: mol_type = other DNA
tgcacatgagtgcttcgat

SEQ ID NO: 1163 - DNA, 21 bp, synthetic construct
  source 1..21: mol_type = other DNA
tgcacatgagtgcagccagat

SEQ ID NO: 1164 - DNA, 19 bp, synthetic construct
  source 1..19: mol_type = other DNA
tgcacaatgagactgcgat

SEQ ID NO: 1165 - DNA, 19 bp, synthetic construct
  source 1..19: mol_type = other DNA
tgcacatggagatactgat

SEQ ID NO: 1166 - DNA, 21 bp, synthetic construct
  source 1..21: mol_type = other DNA
tgcacatgaggctctgatgat

SEQ ID NO: 1167 - DNA, 21 bp, synthetic construct
  source 1..21: mol_type = other DNA
tgcacatgagcaagatgagat

SEQ ID NO: 1168 - DNA, 21 bp, synthetic construct
  source 1..21: mol_type = other DNA
tgcacatgagcatggtctgat

SEQ ID NO: 1169 - DNA, 21 bp, synthetic construct
  source 1..21: mol_type = other DNA
tgcacatgagcatgaggcgat

SEQ ID NO: 1170 - DNA, 19 bp, synthetic construct
  source 1..19: mol_type = other DNA
tgcacaatgatagtgtgat

SEQ ID NO: 1171 - DNA, 21 bp, synthetic construct
  source 1..21: mol_type = other DNA
tgcacatggatagctgcagat

SEQ ID NO: 1172 - DNA, 19 bp, synthetic construct
  source 1..19: mol_type = other DNA
tgcacatgattatgtcgat

SEQ ID NO: 1173 - DNA, 19 bp, synthetic construct
  source 1..19: mol_type = other DNA
tgcacatgatcggactgat

SEQ ID NO: 1174 - DNA, 21 bp, synthetic construct
  source 1..21: mol_type = other DNA
tgcacatgatctgaatgcgat

SEQ ID NO: 1175 - DNA, 21 bp, synthetic construct
  source 1..21: mol_type = other DNA
tgcacatgatcacacaatgat

SEQ ID NO: 1176 - DNA, 21 bp, synthetic construct
  source 1..21: mol_type = other DNA
tgcacaatgatgtcatgtgat

SEQ ID NO: 1177 - DNA, 21 bp, synthetic construct
  source 1..21: mol_type = other DNA
tgcacatggatgacatctgat

SEQ ID NO: 1178 - DNA, 21 bp, synthetic construct
  source 1..21: mol_type = other DNA
tgcacatgattgatcgctgat

SEQ ID NO: 1179 - DNA, 21 bp, synthetic construct
  source 1..21: mol_type = other DNA
tgcacatgatgaatctatgat

SEQ ID NO: 1180 - DNA, 21 bp, synthetic construct
  source 1..21: mol_type = other DNA
tgcacatgatgctcctctgat

SEQ ID NO: 1181 - DNA, 21 bp, synthetic construct
  source 1..21: mol_type = other DNA
tgcacatgatgctcaggagat

SEQ ID NO: 1182 - DNA, 21 bp, synthetic construct
  source 1..21: mol_type = other DNA
tgcacaatgatgctgtatgat

SEQ ID NO: 1183 - DNA, 21 bp, synthetic construct
  source 1..21: mol_type = other DNA
tgcacatggatgcagacagat

SEQ ID NO: 1184 - DNA, 19 bp, synthetic construct
  source 1..19: mol_type = other DNA
tgcacatgcggtatgcgat

SEQ ID NO: 1185 - DNA, 19 bp, synthetic construct
  source 1..19: mol_type = other DNA
tgcacatgcgtcctgtgat

SEQ ID NO: 1186 - DNA, 19 bp, synthetic construct
  source 1..19: mol_type = other DNA
tgcacatgcgtcacctgat

SEQ ID NO: 1187 - DNA, 21 bp, synthetic construct
  source 1..21: mol_type = other DNA
tgcacatgcgtgagcaatgat

SEQ ID NO: 1188 - DNA, 21 bp, synthetic construct
  source 1..21: mol_type = other DNA
tgcacaatgcgtgctgcagat

SEQ ID NO: 1189 - DNA, 21 bp, synthetic construct
  source 1..21: mol_type = other DNA
tgcacatggcgactgcatgat

SEQ ID NO: 1190 - DNA, 19 bp, synthetic construct
  source 1..19: mol_type = other DNA
tgcacatgcggagtgagat

SEQ ID NO: 1191 - DNA, 19 bp, synthetic construct
  source 1..19: mol_type = other DNA
tgcacatgcgaggagcgat

SEQ ID NO: 1192 - DNA, 19 bp, synthetic construct
  source 1..19: mol_type = other DNA
tgcacatgcgagcttcgat

SEQ ID NO: 1193 - DNA, 21 bp, synthetic construct
  source 1..21: mol_type = other DNA
tgcacatgcgagcatggtgat

SEQ ID NO: 1194 - DNA, 21 bp, synthetic construct
  source 1..21: mol_type = other DNA
tgcacaatgcgatcatgagat

SEQ ID NO: 1195 - DNA, 21 bp, synthetic construct
  source 1..21: mol_type = other DNA
tgcacatggcgcgatcatgat

SEQ ID NO: 1196 - DNA, 21 bp, synthetic construct
  source 1..21: mol_type = other DNA
tgcacatgcggcgcagcagat

SEQ ID NO: 1197 - DNA, 19 bp, synthetic construct
  source 1..19: mol_type = other DNA
tgcacatgcgcttacagat

SEQ ID NO: 1198 - DNA, 21 bp, synthetic construct
  source 1..21: mol_type = other DNA
tgcacatgcgctgaatgagat

SEQ ID NO: 1199 - DNA, 19 bp, synthetic construct
  source 1..19: mol_type = other DNA
tgcacaatgcgcacacgat

SEQ ID NO: 1200 - DNA, 21 bp, synthetic construct
  source 1..21: mol_type = other DNA
tgcacatggcgcagtgctgat

SEQ ID NO: 1201 - DNA, 21 bp, synthetic construct
  source 1..21: mol_type = other DNA
tgcacatgcggcatctgcgat

SEQ ID NO: 1202 - DNA, 21 bp, synthetic construct
  source 1..21: mol_type = other DNA
tgcacatgcgcaatgtatgat

SEQ ID NO: 1203 - DNA, 19 bp, synthetic construct
  source 1..19: mol_type = other DNA
tgcacatgctagtggcgat

SEQ ID NO: 1204 - DNA, 21 bp, synthetic construct
  source 1..21: mol_type = other DNA
tgcacatgctatagcaatgat

SEQ ID NO: 1205 - DNA, 19 bp, synthetic construct
  source 1..19: mol_type = other DNA
tgcacaatgctatcgagat

SEQ ID NO: 1206 - DNA, 21 bp, synthetic construct
  source 1..21: mol_type = other DNA
tgcacatggctatgcactgat

SEQ ID NO: 1207 - DNA, 19 bp, synthetic construct
  source 1..19: mol_type = other DNA
tgcacatgcttcgtatgat

SEQ ID NO: 1208 - DNA, 21 bp, synthetic construct
  source 1..21: mol_type = other DNA
tgcacatgctcggctgctgat

SEQ ID NO: 1209 - DNA, 21 bp, synthetic construct
  source 1..21: mol_type = other DNA
tgcacatgctctattgcagat

SEQ ID NO: 1210 - DNA, 21 bp, synthetic construct
  source 1..21: mol_type = other DNA
tgcacatgctctgagcctgat

SEQ ID NO: 1211 - DNA, 21 bp, synthetic construct
  source 1..21: mol_type = other DNA
tgcacaatgctctgcatagat

SEQ ID NO: 1212 - DNA, 21 bp, synthetic construct
  source 1..21: mol_type = other DNA
tgcacatggctcacatatgat

SEQ ID NO: 1213 - DNA, 21 bp, synthetic construct
  source 1..21: mol_type = other DNA
tgcacatgcttcagctcagat

SEQ ID NO: 1214 - DNA, 21 bp, synthetic construct
  source 1..21: mol_type = other DNA
tgcacatgctcaatatctgat

SEQ ID NO: 1215 - DNA, 21 bp, synthetic construct
  source 1..21: mol_type = other DNA
tgcacatgctcatggcgcgat

SEQ ID NO: 1216 - DNA, 19 bp, synthetic construct
  source 1..19: mol_type = other DNA
tgcacaatgctgtagtgat

SEQ ID NO: 1217 - DNA, 19 bp, synthetic construct
  source 1..19: mol_type = other DNA
tgcacatggctgtctcgat

SEQ ID NO: 1218 - DNA, 21 bp, synthetic construct
  source 1..21: mol_type = other DNA
tgcacatgcttgtgtcatgat

SEQ ID NO: 1219 - DNA, 21 bp, synthetic construct
  source 1..21: mol_type = other DNA
tgcacatgctgaatgtgtgat

SEQ ID NO: 1220 - DNA, 21 bp, synthetic construct
  source 1..21: mol_type = other DNA
tgcacatgctgcgttgcagat

SEQ ID NO: 1221 - DNA, 21 bp, synthetic construct
  source 1..21: mol_type = other DNA
tgcacatgctgcgcgaatgat

SEQ ID NO: 1222 - DNA, 21 bp, synthetic construct
  source 1..21: mol_type = other DNA
tgcacaatgctgcacgctgat

SEQ ID NO: 1223 - DNA, 21 bp, synthetic construct
  source 1..21: mol_type = other DNA
tgcacatggctgcagactgat

SEQ ID NO: 1224 - DNA, 21 bp, synthetic construct
  source 1..21: mol_type = other DNA
tgcacatgcttgcatatagat

SEQ ID NO: 1225 - DNA, 19 bp, synthetic construct
  source 1..19: mol_type = other DNA
tgcacatgcacggtgcgat

SEQ ID NO: 1226 - DNA, 19 bp, synthetic construct
  source 1..19: mol_type = other DNA
tgcacatgcactaggtgat

SEQ ID NO: 1227 - DNA, 19 bp, synthetic construct
  source 1..19: mol_type = other DNA
tgcacaatgcactcgagat

SEQ ID NO: 1228 - DNA, 21 bp, synthetic construct
  source 1..21: mol_type = other DNA
tgcacatggcactctcatgat

SEQ ID NO: 1229 - DNA, 19 bp, synthetic construct
  source 1..19: mol_type = other DNA
tgcacatgcaacactagat

SEQ ID NO: 1230 - DNA, 21 bp, synthetic construct
  source 1..21: mol_type = other DNA
tgcacatgcacaagatcagat

SEQ ID NO: 1231 - DNA, 21 bp, synthetic construct
  source 1..21: mol_type = other DNA
tgcacatgcacagaatgtgat

SEQ ID NO: 1232 - DNA, 21 bp, synthetic construct
  source 1..21: mol_type = other DNA
tgcacatgcacagcacctgat

SEQ ID NO: 1233 - DNA, 19 bp, synthetic construct
  source 1..19: mol_type = other DNA
tgcacaatgcacatacgat

SEQ ID NO: 1234 - DNA, 21 bp, synthetic construct
  source 1..21: mol_type = other DNA
tgcacatggcagtcgcatgat

SEQ ID NO: 1235 - DNA, 21 bp, synthetic construct
  source 1..21: mol_type = other DNA
tgcacatgcaagtgtgatgat

SEQ ID NO: 1236 - DNA, 21 bp, synthetic construct
  source 1..21: mol_type = other DNA
tgcacatgcagaagtcatgat

SEQ ID NO: 1237 - DNA, 21 bp, synthetic construct
  source 1..21: mol_type = other DNA
tgcacatgcagagaagatgat

SEQ ID NO: 1238 - DNA, 21 bp, synthetic construct
  source 1..21: mol_type = other DNA
tgcacatgcagagcgcctgat

SEQ ID NO: 1239 - DNA, 21 bp, synthetic construct
  source 1..21: mol_type = other DNA
tgcacaatgcagagcacagat

SEQ ID NO: 1240 - DNA, 21 bp, synthetic construct
  source 1..21: mol_type = other DNA
tgcacatggcagatatgtgat

SEQ ID NO: 1241 - DNA, 21 bp, synthetic construct
  source 1..21: mol_type = other DNA
tgcacatgcaagatctcagat

SEQ ID NO: 1242 - DNA, 21 bp, synthetic construct
  source 1..21: mol_type = other DNA
tgcacatgcagaatgtgcgat

SEQ ID NO: 1243 - DNA, 21 bp, synthetic construct
  source 1..21: mol_type = other DNA
tgcacatgcagatggcgagat

SEQ ID NO: 1244 - DNA, 21 bp, synthetic construct
  source 1..21: mol_type = other DNA
tgcacatgcagcgctaatgat

SEQ ID NO: 1245 - DNA, 21 bp, synthetic construct
  source 1..21: mol_type = other DNA
tgcacaatgcagcacgatgat

SEQ ID NO: 1246 - DNA, 21 bp, synthetic construct
  source 1..21: mol_type = other DNA
tgcacatggcatactgctgat

SEQ ID NO: 1247 - DNA, 21 bp, synthetic construct
  source 1..21: mol_type = other DNA
tgcacatgcaatacatgagat

SEQ ID NO: 1248 - DNA, 21 bp, synthetic construct
  source 1..21: mol_type = other DNA
tgcacatgcataagagctgat

SEQ ID NO: 1249 - DNA, 21 bp, synthetic construct
  source 1..21: mol_type = other DNA
tgcacatgcatataatgcgat

SEQ ID NO: 1250 - DNA, 21 bp, synthetic construct
  source 1..21: mol_type = other DNA
tgcacatgcatcgcgccagat

SEQ ID NO: 1251 - DNA, 21 bp, synthetic construct
  source 1..21: mol_type = other DNA
tgcacaatgcatctatatgat

SEQ ID NO: 1252 - DNA, 21 bp, synthetic construct
  source 1..21: mol_type = other DNA
tgcacatggcatctgtgtgat

SEQ ID NO: 1253 - DNA, 21 bp, synthetic construct
  source 1..21: mol_type = other DNA
tgcacatgcaatcacatcgat

SEQ ID NO: 1254 - DNA, 19 bp, synthetic construct
  source 1..19: mol_type = other DNA
tgcacatgcatggtacgat

SEQ ID NO: 1255 - DNA, 21 bp, synthetic construct
  source 1..21: mol_type = other DNA
tgcacatgcatgtggatagat

SEQ ID NO: 1256 - DNA, 21 bp, synthetic construct
  source 1..21: mol_type = other DNA
tgcacatgcatgcgaggagat

SEQ ID NO: 1257 - DNA, 12 bp, synthetic construct
  source 1..12: mol_type = other DNA
tatggtcgtcga